(19) United States
(12) Patent Application Publication
(10) Pub. No.: US 2025/0248999 A1
(43) Pub. Date: Aug. 7, 2025

(54) BAVDEGALUTAMIDE AND COMBINATIONS THEREOF FOR USE IN TREATING PROSTATE CANCER

(71) Applicant: Arvinas Operations, Inc., New Haven, CT (US)

(72) Inventors: Sarah Deborah CHIRNOMAS, Woodbridge, CT (US); Cecile Bulanhagui MATHER, Randolph, NH (US); Janaki PARAMESWARAN, Princeton, NJ (US); Sanghyun LEE, Chesterbrook, PA (US); Ronald PECK, Cheshire, CT (US); Lawrence B. SNYDER, Killingworth, CT (US); Marcia Dougan MOORE, Suffield, CT (US)

(21) Appl. No.: 18/857,114

(22) PCT Filed: Apr. 21, 2023
(86) PCT No.: PCT/US2023/019496

§ 371 Date: Oct. 15, 2024

Related U.S. Application Data
(60) Provisional application No. 63/333,491, filed on Apr. 21, 2022.

(51) Int. Cl. A61K 31/501; A61K 31/58; A61P 35/00

(52) U.S. Cl. CPC A61K 31/501; A61K 31/58; A61P 35/00

(57) ABSTRACT

This disclosure pertains to methods of treating prostate cancer in a subject, including, for example, metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), and metastatic castrate-sensitive prostate cancer naïve to novel hormonal agents (NHA), wherein the method comprises: (i) administering a compound of Formula (I), (I), or a pharmaceutically acceptable salt thereof, wherein R¹, R², R³, X¹, X², X³, X⁴, and n are defined herein; and (ii) abiraterone, a pharmaceutically acceptable salt of abiraterone, abiraterone acetate, or a pharamaceutically acceptable sale of abiraterone acetate.

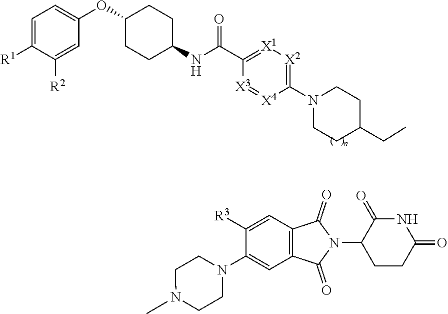

### CROSS-REFERENCE TO RELATED APPLICATIONS

This application claims priority to, and the benefit of, U.S. Provisional Application No. 63/333,491, filed on Apr. 21, 2022, which is incorporated by reference herein in their entirety for all purposes.

### FIELD OF THE DISCLOSURE

This application relates to treating prostate cancer, including metastatic and/or castrate-resistant prostate cancer, comprising administering a compound of Formula (I) and abiraterone, abiraterone acetate, or a pharmaceutically acceptable salt thereof, to a subject in need of treatment.

### BACKGROUND OF THE DISCLOSURE

Androgen Receptor (AR) belongs to a nuclear hormone receptor family that is activated by androgens, such as testosterone and dihydrotestosterone (Pharmacol. Rev. 2006, 58(4), 782-97; Vitam. Horn. 1999, 55:309-52.). In the absence of androgens, AR is bound by Heat Shock Protein 90 (Hsp90) in the cytosol. When an androgen binds AR, its conformation changes to release AR from Hsp90 and to expose the Nuclear Localization Signal (NLS). The latter enables AR to translocate into the nucleus where AR acts as a transcription factor to promote gene expression responsible for male sexual characteristics (Endocr. Rev. 1987, 8(1):1-28; Mol. Endocrinol. 2002, 16(10), 2181-7). AR deficiency leads to Androgen Insensitivity Syndrome, formerly termed testicular feminization.

While AR is responsible for development of male sexual characteristics, it is also a well-documented oncogene in certain forms of cancers including prostate cancers (Endocr. Rev. 2004, 25(2), 276-308). A commonly measured target gene of AR activity is the secreted Prostate Specific Antigen (PSA) protein. The current treatment regimen for prostate cancer involves inhibiting the androgen-AR axis by two methods. The first approach relies on reduction of androgens, while the second strategy aims to inhibit AR function (Nat. Rev. Drug Discovery, 2013, 12, 823-824). Despite the development of effective targeted therapies, most patients develop resistance and the disease progresses. An alternative approach for the treatment of prostate cancer involves eliminating the AR protein. Because AR is a critical driver of tumorigenesis in many forms of prostate cancers, its elimination should lead to a therapeutically beneficial response. There exists an ongoing need in the art for effective treatments for diseases, especially cancer, prostate cancer, and Kennedy's Disease. However, non-specific effects, and the inability to target and modulate certain classes of proteins altogether, such as transcription factors, remain as obstacles to the development of effective anticancer agents. As such, small molecule therapeutic agents that leverage or potentiate cereblon's substrate specificity and, at the same time, are “tunable” such that a wide range of protein classes can be targeted and modulated with specificity would be very useful as a therapeutic.

### SUMMARY OF THE DISCLOSURE

In one aspect, this disclosure pertains to a method of treating prostate cancer in a subject in need thereof, comprising: (i) oral, once daily administration of about 35 mg to about 500 mg of a compound of Formula (I-g):

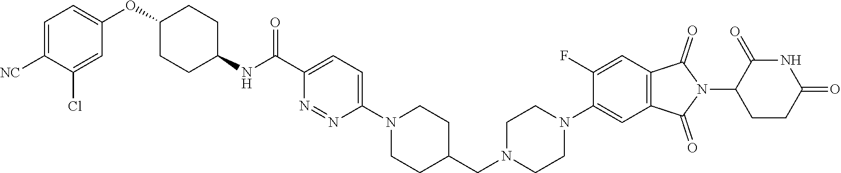 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and (ii) oral, once daily administration of about 1000 mg abiraterone, a pharmaceutically acceptable salt of abiraterone, abiraterone acetate, or a pharmaceutically acceptable salt of abiraterone acetate.

In one aspect, this disclosure pertains to a method of treating prostate cancer in a subject in need thereof, comprising: (i) oral, once daily administration of about 35 mg to about 500 mg of compound of Formula (I-g):

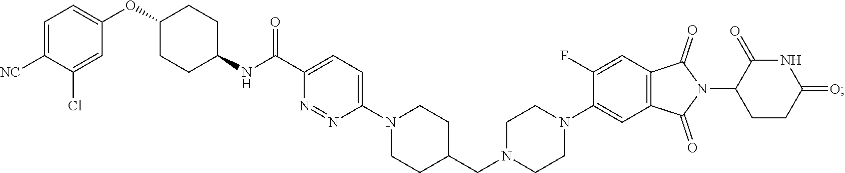 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and (ii) oral, once daily administration of about 1000 mg abiraterone acetate.

In some embodiments, the methods of the disclosure further comprise administering a corticosteroid to the subject.

In some embodiments, about 0.5 mg, about 1 mg, about 1.5 mg, about 2 mg, about 2.5 mg, about 3 mg, about 3.5 mg, about 4 mg, about 4.5 mg, or about 5 mg of the corticosteroid is administered to the subject.

In some embodiments, the corticosteroid is orally administered to the subject once daily.

In some embodiments, the corticosteroid is orally administered to the subject twice daily.

In some embodiments, the corticosteroid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclomethasone.

In some embodiments, the corticosteroid is prednisone.

In some embodiments, the corticosteroid is prednisolone.

In some embodiments, 1,000 mg of abiraterone acetate is orally administered to the subject once daily.

In some embodiments, about 35 mg to about 100 mg of the compound of Formula (I-g) is orally administered to the subject once daily.

In some embodiments, about 100 mg to about 200 mg of the compound of Formula (I-g) is orally administered to the subject once daily.

In some embodiments, about 200 mg to about 300 mg of the compound of Formula (I-g) is orally administered to the subject once daily.

In some embodiments, about 300 mg to about 400 mg of the compound of Formula (I-g) is orally administered to the subject once daily.

In some embodiments, about 400 mg to about 500 mg of the compound of Formula (I-g) is orally administered to the subject once daily.

In some embodiments, about 35 mg of the compound of Formula (I-g) is orally administered to the subject once daily.

In some embodiments, about 100 mg of the compound of Formula (I-g) is orally administered to the subject once daily.

In some embodiments, about 140 mg of the compound of Formula (I-g) is orally administered to the subject once daily.

In some embodiments, about 150 mg of the compound of Formula (I-g) is orally administered to the subject once daily.

In some embodiments, about 200 mg of the compound of Formula (I-g) is orally administered to the subject once daily.

In some embodiments, about 250 mg of the compound of Formula (I-g) is orally administered to the subject once daily.

In some embodiments, about 300 mg of the compound of Formula (I-g) is orally administered to the subject once daily.

In some embodiments, about 350 mg of the compound of Formula (I-g) is orally administered to the subject once daily.

In some embodiments, about 400 mg of the compound of Formula (I-g) is orally administered to the subject once daily.

In some embodiments, about 420 mg of the compound of Formula (I-g) is orally administered to the subject once daily.

In some embodiments, about 450 mg of the compound of Formula (I-g) is orally administered to the subject once daily.

In some embodiments, about 500 mg of the compound of Formula (I-g) is orally administered to the subject once daily.

In some embodiments, the compound of Formula (I-g) is formulated as a tablet.

In some embodiments, the tablet comprises the compound of Formula (I-g) and, optionally, one or more of the following: emulsifier; surfactant; binder; disintegrant; glidant; and lubricant.

In some embodiments, the subject is in a fed state.

In some embodiments, the subject is in a fasted state.

In some embodiments, the prostate cancer is adenocarcinoma of the prostate.

In some embodiments, the prostate cancer is metastatic prostate cancer.

In some embodiments, the prostate cancer is castrate-resistant prostate cancer.

In some embodiments, the prostate cancer is metastatic castrate-resistant prostate cancer.

In some embodiments, the prostate cancer is progressive metastatic castrate-resistant prostate cancer.

In some embodiments, the prostate cancer is castrate-sensitive prostate cancer.

In some embodiments, the prostate cancer is metastatic castrate-sensitive prostate cancer.

In some embodiments, the prostate cancer is prostate cancer naïve to novel hormonal agents (NHA). In some embodiments, the prostate cancer naïve to novel hormonal agents (NHA) has not been previously treated with a second generation antiandrogen.

In some embodiments, the prostate cancer is metastatic prostate cancer naïve to novel hormonal agents (NHA). In some embodiments, the metastatic prostate cancer naïve to novel hormonal agents (NHA) has not been previously treated with a second generation antiandrogen.

In some embodiments, the prostate cancer is castrate-resistant prostate cancer naïve to novel hormonal agents (NHA). In some embodiments, the castrate-resistant prostate cancer naïve to novel hormonal agents (NHA) has not been previously treated with a second generation antiandrogen.

In some embodiments, the prostate cancer is castrate-sensitive prostate cancer naïve to novel hormonal agents (NHA). In some embodiments, the castrate-sensitive prostate cancer naïve to novel hormonal agents (NHA) has not been previously treated with a second generation antiandrogen.

In some embodiments, the prostate cancer is metastatic castrate-resistant prostate cancer naïve to novel hormonal agents (NHA). In some embodiments, the metastatic castrate-resistant prostate cancer naïve to novel hormonal agents (NHA) has not been previously treated with a second generation antiandrogen.

In some embodiments, the prostate cancer is metastatic castrate-sensitive prostate cancer naïve to novel hormonal agents (NHA). In some embodiments, the metastatic castrate-sensitive prostate cancer naïve to novel hormonal agents (NHA) has not been previously treated with a second generation antiandrogen.

In some embodiments, the prostate cancer has not been previously treated with an androgen biosynthesis inhibitor or an androgen receptor blocker.

In some embodiments, the prostate cancer has not been previously treated with abiraterone acetate.

In some embodiments, the prostate cancer has not been previously treated with an androgen receptor blocker selected from enzalutamide, darolutamide, and apalutamide.

In some embodiments, the prostate cancer has not been previously treated with enzalutamide.

In some embodiments, the subject has not been previously administered an androgen biosynthesis inhibitor or an androgen receptor blocker.

In some embodiments, the subject has not been previously administered abiraterone acetate.

In some embodiments, the subject has not been previously administered an androgen receptor blocker selected from enzalutamide, darolutamide, and apalutamide.

In some embodiments, the subject has not been previously administered enzalutamide.

In one aspect, this disclosure pertains to a combined preparation of: (i) the compound of Formula (I-g):

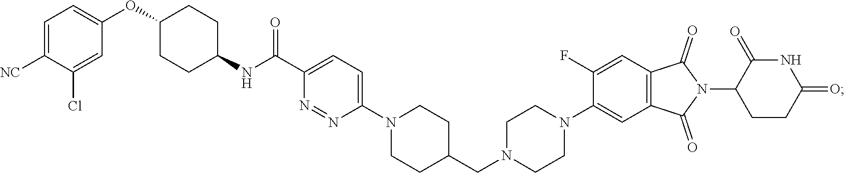 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and (ii) abiraterone acetate, or a pharmaceutically acceptable salt thereof; for simultaneous, separate or sequential use in a method of treating prostate cancer in a subject. In some embodiments, the method comprises: (i) oral, once daily administration of about 35 mg to about 500 mg of the compound of Formula (I-g) or the pharmaceutically acceptable salt thereof; and (ii) oral, once daily administration of about 1000 mg abiraterone acetate, or the pharmaceutically acceptable salt thereof. In some embodiments, the method comprises: (i) oral, once daily administration of about 35 mg to about 500 mg of the compound of Formula (I-g); and (ii) oral, once daily administration of 1000 mg abiraterone acetate.

In one aspect, this disclosure pertains to a compound of Formula (I-g):

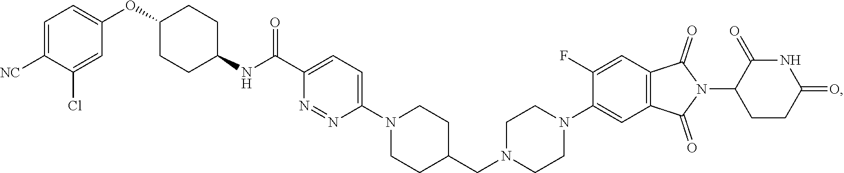 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

for use in a method of treating prostate cancer in a subject, wherein the method comprises: (i) oral, once daily administration of about 35 mg to about 500 mg of the compound of Formula (I-g) or the pharmaceutically acceptable salt thereof; and (ii) oral, once daily administration of about 1000 mg abiraterone acetate, or a pharmaceutically acceptable salt thereof.

In one aspect, this disclosure pertains to a compound of Formula (I-g):

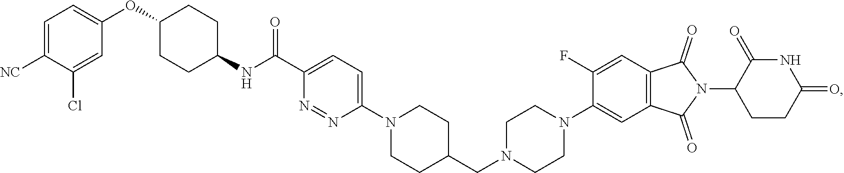 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

for use in a method of treating prostate cancer in a subject, wherein the method comprises: (i) oral, once daily administration of about 35 mg to about 500 mg of the compound of Formula (I-g); and (ii) oral, once daily administration of 1000 mg abiraterone acetate.

In one aspect, this application pertains to a method of treating prostate cancer in a subject in need thereof, the method comprising administering to the subject:
(i) a compound of Formula (I), or a pharmaceutically acceptable salt, enantiomer, stereoisomer, solvate, polymorph, isotopic derivative, or prodrug thereof, wherein the compound of Formula (I) is:

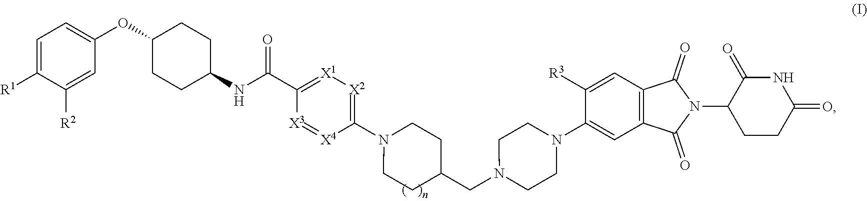

wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and
   (ii) abiraterone.

In some embodiments, for any of the methods of the present disclosure, the compound of Formula (I) is:

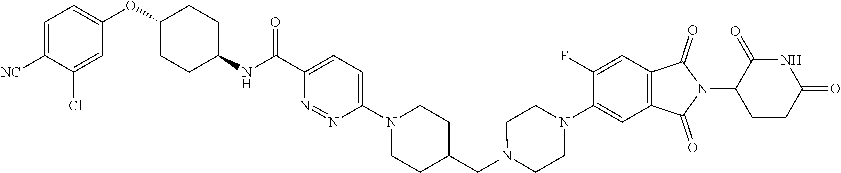 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

In some embodiments, for any of the methods of the present disclosure, the abiraterone is abiraterone acetate.

### BRIEF DESCRIPTION OF THE FIGURES

FIG. 1 is a dose-response curve comparing the in vitro inhibitory effect of VCaP proliferation of Compound (I-g) with enzalutamide.

FIG. 2 is Western Blot experiment that shows the reduction of AR in VCaP tumor cells in response to treatment with Compound (I-g) at concentrations of 0.03 nM, 0.1 nM, 0.3 nM, 1 nM, 3 nM, 10 nM, 30 nM, 100 nM, and 300 nM.

FIG. 3 is series of line graphs summarizing animal experiments performed in a castrated VCaP xenograft model. Compound (I-g) was administered orally, once daily at doses of 0.1 mg/kg (mpk), 0.3 mg/kg, 1 mg/kg, and 3 mg/kg. Enzalutamide (20 mg/kg) and vehicle were also used as control groups.

FIG. 4 is series of line graphs summarizing animal experiments performed in an intact (non-castrated) VCaP xenograft model. Compound (I-g) was administered orally, once daily at doses of 1 mg/kg, 3 mg/kg, and 10 mg/kg. Enzalutamide (20 mg/kg) and vehicle were also used as control groups.

FIG. 5 is series of line graphs summarizing animal experiments performed in an enzalutamide resistant VCaP xenograft model. Compound (I-g) was administered orally, once daily at doses of 3 mg/kg and 10 mg/kg. Enzalutamide (20 mg/kg) and vehicle were also used as control groups.

FIG. 6 is Western Blot experiment that shows the reduction of AR in enzalutamide-resistant VCaP tumors in response to dosing with Compound (I-g) at 10 mg/kg and 3 mg/kg (oral, once daily).

FIG. 7 is a series of line graphs which provides a representation of the mean concentrations of Compound (I-g) over a 24 hour time period after dosing on day 15 for all three tested doses (35 mg/day, 70 mg/day, and 140 mg/day, oral administration).

FIG. 8 is a bar graph (aka, waterfall plot) showing the best percent change in Prostate-Specific Antigen (PSA) test results in 20 patients with metastatic castration resistant prostate cancer (mCRPC) receiving Compound (I-g). Each bar represents the best percent change in plasma PSA from pre-treatment levels of a single patient. Patients received either 35 mg/day, 70 mg/day, 140 mg/day, or 280 mg/day of Compound (I-g), as indicated in the legend.

FIG. 9 is a bar graph (aka, waterfall plot) showing best percent change in Prostate-Specific Antigen (PSA) test results in 12 patients with mCRPC receiving ≥140 mg daily dose of Compound (I-g), as well as the molecular status of the AR gene or protein present in circulating tumor DNA or circulating tumor cells, respectively, isolated from each patient. Each bar represents the best percent change in plasma PSA from pre-treatment levels of a single patient. AR-V7 is a splice variant of AR. Amplif refers to amplification of the AR gene.

FIG. 10 summarizes the key features of one patient (“patient 19”) who received a 140 mg/day dose of Compound (I-g). This patient corresponds with the second bar from right in both FIG. 8 and FIG. 9.

FIG. 11A summarizes the key features of one patient (“patient 20”) who received a 140 mg/day dose of Compound (I-g). This patient corresponds with rightmost bar in FIG. 8 and FIG. 9. FIG. 11B shows a CT scan of the patient 20's tumor prior to treatment. FIG. 11C shows a CT scan of the patient 20's tumor after 4 cycles, showing the RECIST response.

FIG. 12 is a representation of the Mean Day 15 AUC₀₋₂₄ (ng*hr/mL) of Compound (I-g) over a 24 hour time period after dosing on day 15 for all four tested doses (35 mg/day, 70 mg/day, 140 mg/day, and 280 mg/day, oral administration).

FIG. 13 is a series of line graphs which provides a representation of the mean concentrations of Compound (I-g) over a 24 hour time period after dosing on day 15 for all four tested doses (in order from lowest to highest on the y-axis—35 mg/day, 70 mg/day, 140 mg/day, and 280 mg/day, oral administration).

FIG. 14 is a series of line graphs summarizing animal experiments performed in surgically castrated CB17/scid male mice with subcutaneously implanted VCaP tumors in the dorsal flank. Compound (I-g) (3 mg/kg), abiraterone (100 mg/kg), or a combination of both agents was administered by oral gavage once daily. Vehicle was used as a control group.

FIG. 15 is a series of lines summarizing a three-stage study in surgically castrated CB17/scid male mice with subcutaneously implanted VCaP tumors in the dorsal flank.

FIG. 16 is a series of line graphs summarizing the changes in the animal's body weights.

FIG. 17 is a Western Blot experiment that shows AR levels in VCaP tumors harvested from castrated CB17/scid mice after treatment with Compound (I-g) at 3 mg/kg (oral, once daily) or a combination of Compound (I-g) at 3 mg/kg (oral, once daily) and abiraterone at 100 mg/kg, (oral, once daily).

FIG. 18 is a quantification of the Western Blot Experiment in FIG. 17.

FIG. 19A is a series of line graphs summarizing the efficacy of Compound (I-g) in a prostate cancer patient-derived xenograft mouse model. Compound (I-g) was administered orally, at a 10 mg/kg dose. Enzalutamide (20 mg/kg) and vehicle were also used as control groups. FIG. 19B shows the reduction of plasma PSA levels following treatment with enzalutamide (20 mg/kg) and Compound (I-g) (10 mg/kg) compared to vehicle.

### DETAILED DESCRIPTION

### Definitions

“Halogen” or “halo” refers to fluorine (F), chlorine (Cl), bromine (Br), or iodine (I).

“C₁-C₆ alkyl” refers to a straight or branched chain saturated hydrocarbon containing 1-6 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Pharmaceutically acceptable salt”, as used herein with respect to a compound of Formula (I), means a salt form of a compound of Formula (I) as well as hydrates of the salt form with one or more water molecules present. Such salt and hydrated forms retain the biological activity of a compound of Formula (I) and are not biologically or otherwise undesirable, i.e., exhibit minimal, if any, toxicological effects. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “isomer” refers to salts and/or compounds that have the same composition and molecular weight but differ in physical and/or chemical properties. The structural difference may be in constitution (geometric isomers) or in the ability to rotate the plane of polarized light (stereoisomers). With regard to stereoisomers, the salts of a compound of Formula (I) may have one or more asymmetric carbon atom and may occur as racemates, racemic mixtures and as individual enantiomers or diastereomers.

The compounds of Formula (I) may exist in unsolvated as well as solvated forms such as, for example, hydrates.

“Solvate” means a solvent addition form that contains either a stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the invention.

When a compound is crystallized from a solution or slurry, it can be crystallized in a different arrangement lattice of spaces (this property is called “polymorphism”) to form crystals with different crystalline forms, each of which is known as “polymorphs”. “Polymorph”, as used herein, refers to a crystal form of a compound of Formula (I) where the molecules are localized in the three-dimensional lattice sites. Different polymorphs of the compound of Formula (I) may be different from each other in one or more physical properties, such as solubility and dissolution rate, true specific gravity, crystal form, accumulation mode, flowability and/or solid state stability, etc.

“Isotopic derivative”, as referred to herein, relates to a compound of Formula (I) that is isotopically enriched or labelled (with respect to one or more atoms of the compound) with one or more stable isotopes. Thus, in this application, the compounds of Formula (I) include, for example, compounds that are isotopically enriched or labelled with one or more atoms such as deuterium.

The term “pharmaceutically acceptable prodrugs” as used herein refers to those prodrugs of the compounds of Formula (I) which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention.

“Prodrug”, as used herein means a compound which is convertible in vivo by metabolic means (e.g., by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 1 13-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing, and methods of treating disorders through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities. Combinations of substituents and variables envisioned by this invention are only those that result in the formation of stable compounds.

Prostate cancer is the uncontrolled growth of cancerous cells in the prostate gland. In some embodiments, the prostate cancer is metastatic prostate cancer, castrate-resistant prostate cancer, metastatic castrate-resistant prostate cancer, castrate-sensitive prostate cancer, metastatic castrate-sensitive prostate cancer, prostate cancer naïve to novel hormonal agents (NHA), castrate-sensitive prostate cancer naïve to novel hormonal agents (NHA), castrate-resistant prostate cancer naïve to novel hormonal agents (NHA), metastatic prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA).

Metastatic prostate cancer, or metastases, refers to prostate cancer that has spread beyond the prostate to other parts of the body, e.g., bones, lymph nodes, liver, lungs, brain.

Castrate-resistant prostate cancer or castration-resistant prostate cancer (or prostate cancer that is castrate- or castration-resistant) is a type of prostate cancer that keeps growing even when the amount of testosterone in the body is reduced to very low levels.

Metastatic castrate-resistant prostate cancer is a type of prostate cancer that has metastasized and continues to grow even when the amount of testosterone in the body is reduced to very low levels.

Castrate-sensitive prostate cancer or castration-sensitive prostate cancer (CSPC), or prostate cancer that is castrate- or castration-sensitive, is prostate cancer that can be controlled by reducing the amount of androgens (male hormones) in the body (e.g., through castration) and/or prostate cancer that requires androgens to grow and stops growing when androgens are not present. CSPC is also referred to as androgen-dependent prostate cancer, androgen-sensitive prostate cancer, or hormone-sensitive prostate cancer (HSPC).

Metastatic castrate-sensitive prostate cancer is a type of castrate-sensitive prostate cancer that has metastasized and requires androgens to grow, or can be controlled by reducing the amount of androgens in the body (e.g., through castration).

Prostate cancer naïve to novel hormonal agents (NHA) is prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Metastatic prostate cancer naïve to novel hormonal agents (NHA) is metastatic prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Castrate-resistant prostate cancer naïve to novel hormonal agents (NHA) is castrate-resistant prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Castrate-sensitive prostate cancer naïve to novel hormonal agents (NHA) is castrate-sensitive prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Metastatic castrate-resistant prostate cancer naïve to novel hormonal agents (NHA) is metastatic castrate-resistant prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Metastatic castrate-sensitive prostate cancer naïve to novel hormonal agents (NHA) is metastatic castrate-sensitive prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

As used herein, “treating” describes the management and care of a subject for the purpose of combating a disease, condition, or disorder and includes decreasing or alleviating the symptoms or complications, or eliminating the disease, condition or disorder.

As used herein, “preventing” describes stopping the onset of the symptoms or complications of the disease, condition or disorder.

“Administration” refers to introducing an agent, such as a compound of Formula (I) into a subject. The related terms “administering” and “administration of” (and grammatical equivalents) refer both to direct administration, which may be administration to a subject by a medical professional or by self-administration by the subject, and/or to indirect administration, which may be the act of prescribing a drug. For example, a physician who instructs a patient to self-administer a drug and/or provides a patient with a prescription for a drug is administering the drug to the patient.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, a compound of Formula (I), as described herein, is co-administered in combination with abiraterone, abiraterone acetate, or a pharmaceutically acceptable salt thereof. In particularly preferred aspects, the co-administration of a compound of Formula (I), as described herein, and abiraterone, abiraterone acetate, or a pharmaceutically acceptable salt thereof, results in synergistic activity and/or therapy, including anticancer activity.

“Therapeutically effective amount”, as used herein means an amount of the free base of a compound of Formula (I) that is sufficient to treat, ameliorate, or prevent a specified disease (e.g., prostate cancer), disease symptom, disorder or condition, or to exhibit a detectable therapeutic or inhibitory effect. The effect can be detected by any assay method known in the art. The effective amount for a particular subject may depend upon the subject's body weight, size, and health; the nature and extent of the condition; and whether additional therapeutics are to be administered to the subject. Therapeutically effective amounts for a given situation can be determined by routine experimentation that is within the skill and judgment of the clinician.

“Cmax”, as used herein, refers to the observed maximum (peak) plasma concentration of a specified compound in the subject after administration of a dose of that compound to the subject.

“AUC”, as used herein, refers to the total area under the plasma concentration-time curve, which is a measure of exposure to a compound of interest, and is the integral of the concentration-time curve after a single dose or at steady state. AUC is expressed in units of ng*H/mL (ng×H/mL).

“AUCₜₐᵤ”, as used herein, refers to the AUC from 0 hours to the end of a dosing interval.

“AUC₀₋₂₄” means the AUC from 0 hours to 24 hours after administration of a single dose.

“Controlled release” or “CR” as used herein with respect to an oral dosage form of the disclosure means that a compound of Formula (I) is released from the dosage form according to a pre-determined profile that may include when and where release occurs after oral administration and/or a specified rate of release over a specified time period.

“Controlled release agent” as used herein with respect to an oral dosage form of the disclosure refers to one or more substances or materials that modulate release of a compound of Formula (I) from the dosage form. Controlled release agents may be materials which are organic or inorganic, naturally occurring or synthetic, such as polymeric materials, triglycerides, derivatives of triglycerides, fatty acids and salts of fatty acids, talc, boric acid and colloidal silica.

“Enteric coating” as used herein with respect to a dosage form of the disclosure refers to a pH-dependent material that surrounds a core comprising a compound of Formula (I) and which remains substantially intact in the acid environment of the stomach, but which dissolves in the pH environment of the intestines.

“Gastro-resistant” or “GR” as applied to a CR oral dosage form described herein means that release of a compound of Formula (I) in the stomach of a subject shall not exceed 5%, 2.5%, 1% or 0.5% of the total amount of the compound of Formula (I) in the dosage form.

“Oral dosage form” as used herein refers to a pharmaceutical drug product that contains a specified amount (dose) of a compound of Formula (I) as the active ingredient, or a pharmaceutically acceptable salt and/or solvate thereof, and inactive components (excipients), formulated into a particular configuration that is suitable for oral administration, such as a tablet or capsule. In some embodiments, the compositions are in the form of a tablet that can be scored.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

Abiraterone acetate, i.e.: [(3S,8R,9S,10R,13S,14S)-10,13-dimethyl-17-pyridin-3-yl-2,3,4,7,8,9,11,12,14,15-decahydro-1H-cyclopenta[a]phenanthren-3-yl]acetate, is a commercially available drug developed by Janssen and sold under the brand name Zytiga® that is indicated for use in combination with prednisone for the treatment of patients with metastatic castration-resistant prostate cancer. The structure of abiraterone acetate is:

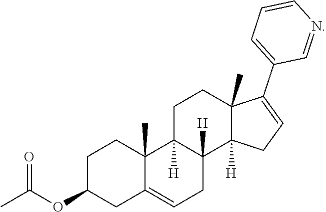

Abiraterone, i.e., (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol, is the active metabolite of abiraterone acetate, and has the following structure:

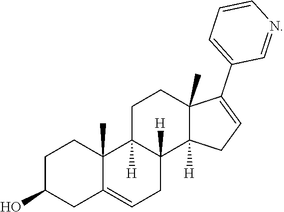

The term “about” as part of a quantitative expression such as “about X”, includes any value that is 10% higher or lower than X, and also includes any numerical value that falls between X−10% and X+10%. Thus, for example, a weight of about 40 g includes a weight of between 36 to 44 g.

“Comprising” or “comprises” as applied to a particular dosage form, composition, use, method or process described or claimed herein means that the dosage form, composition, use, method, or process includes all of the recited elements in a specific description or claim, but does not exclude other elements. “Consists essentially of” and “consisting essentially of” means that the described or claimed composition, dosage form, method, use, or process does not exclude other materials or steps that do not materially affect the recited physical, pharmacological, pharmacokinetic properties or therapeutic effects of the composition, dosage form, method, use, or process. “Consists of” and “consisting of” means the exclusion of more than trace elements of other ingredients and substantial method or process steps.

The ECOG Scale of Performance Status was developed by the Eastern Cooperative Oncology Group as standard criteria for measuring how the disease impacts a patient's daily living abilities. It describes a patient's level of functioning in terms of their ability to care for themself, daily activity, and physical ability (walking, working, etc.). Patients are categorized on a scale of 1-5:

| GRADE | ECOG PERFORMANCE STATUS |
|---|---|
| 0 | Fully active, able to carry on all pre-disease performance without restriction |
| 1 | Restricted in physically strenuous activity but ambulatory and able to carry |
| | out work of a light or sedentary nature, e.g., light house work, office work |
| 2 | Ambulatory and capable of all selfcare but unable to carry out any work |
| | activities; up and about more than 50% of waking hours |
| 3 | Capable of only limited selfcare; confined to bed or chair more than |
| | 50% of waking hours |
| 4 | Completely disabled; cannot carry on any selfcare; totally confined |
| | to bed or chair |
| 5 | Dead |

“Fasted condition” or “fasted state” as used to describe a subject means the subject has not eaten for at least 4 hours before a time point of interest, such as the time of administering a compound of Formula (I). In an embodiment, a subject in the fasted state has not eaten for at least any of 6, 8, 10 or 12 hours prior to administration of a compound of Formula (I).

“Fed condition” or “fed state” as used to describe a subject herein means the subject has eaten less than 4 hours before a time point of interest, such as the time of administering a compound of Formula (I). In an embodiment, a subject in the fed state has eaten within 3, 2, 1, or 0.5 hours prior to administration of a compound of Formula (I).

Corticosteroids are a class of steroid hormones that include both steroid hormones produced in humans and other vertebrates and synthetic derivatives or analogues of the same. In some embodiments, the corticosteroid is a glucocorticoid. In some embodiments, the corticosteroid is a mineralocorticoid. In some embodiments, the corticosteroid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclometasone.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The terms “patient” and “subject” are used interchangeably herein, and refer to a mammal, e.g., a human, mouse, rat, guinea pig, dog, cat, horse, cow, pig, or non-human primate, such as a monkey, chimpanzee, baboon or rhesus.

In some embodiments, the subject is a human.

In some embodiments, the subject is a human who has been diagnosed with prostate cancer.

In some embodiments, the subject is a human who has been diagnosed with metastatic prostate cancer.

In some embodiments, the subject is a human who has been diagnosed with castrate-resistant prostate cancer.

In some embodiments, the subject is a human who has been diagnosed with metastatic, castrate-resistant prostate cancer.

In some embodiments, the subject is a human who has been diagnosed with castrate-sensitive prostate cancer.

In some embodiments, the subject is a human who has been diagnosed with metastatic castrate-sensitive prostate cancer.

In some embodiments, the subject is a human who has been diagnosed with prostate cancer naïve to novel hormonal agents (NHA).

In some embodiments, the subject is a human who has been diagnosed with castrate-sensitive prostate cancer naïve to novel hormonal agents (NHA).

In some embodiments, the subject is a human who has been diagnosed with castrate-resistant prostate cancer naïve to novel hormonal agents (NHA)

In some embodiments, the subject is a human who has been diagnosed with metastatic prostate cancer naïve to novel hormonal agents (NHA).

In some embodiments, the subject is a human who has been diagnosed with metastatic castrate-resistant prostate cancer naïve to novel hormonal agents (NHA).

In some embodiments, the subject is a human who has been diagnosed with metastatic castrate-sensitive prostate cancer naïve to novel hormonal agents (NHA).

In some embodiments, the subject has not been previously treated with enzalutamide, apalutamide, darolutamide, or experimental therapies (e.g., protein degraders or inhibitors) directed at the AR.

In some embodiments, the subject has been previously treated with enzalutamide, apalutamide, darolutamide, or experimental therapies (e.g., protein degraders or inhibitors) directed at the AR.

In some embodiments, the subject has not been treated with any chemotherapy, investigational agents, immunotherapy, or hormonal therapy other than gonadotropin-releasing hormone agonist within 28 days of the start of treatment on protocol.

### Compounds of Formula (I)

In one aspect, the application pertains to the methods of treating and/or preventing cancer comprising the administration of a compound of Formula (I) (preferably, the compound of Formula (I-g)) to subject in need thereof.

In one aspect, the application pertains to the use of a compound of Formula (I) (preferably, the compound of Formula (I-g)) in the treatment and/or prevention of prostate cancer.

In one aspect, the application pertains to the use of a compound of Formula (I) (preferably, the compound of Formula (I-g)) in the manufacture of a medicament for the treatment and/or prevention of prostate cancer.

In one aspect, the application pertains to the methods of treating and/or preventing cancer comprising the administration of a compound of Formula (I) (preferably, the compound of Formula (I-g)) and abiraterone, abiraterone acetate, or a pharmaceutically acceptable salt thereof to subject in need thereof.

In one aspect, the application pertains to the use of a compound of Formula (I) (preferably, the compound of Formula (I-g)) and abiraterone, abiraterone acetate, or a pharmaceutically acceptable salt thereof in the treatment and/or prevention of prostate cancer.

In one aspect, the application pertains to the use of a compound of Formula (I) (preferably, the compound of Formula (I-g)) and abiraterone, abiraterone acetate, or a pharmaceutically acceptable salt thereof in the manufacture of a medicament for the treatment and/or prevention of prostate cancer.

In one aspect, the application pertains to the methods of treating and/or preventing cancer comprising the administration of a compound of Formula (I) (preferably, the compound of Formula (I-g)) and abiraterone acetate to subject in need thereof.

In one aspect, the application pertains to the use of a compound of Formula (I) (preferably, the compound of Formula (I-g)) and abiraterone acetate in the treatment and/or prevention of prostate cancer.

In one aspect, the application pertains to the use of a compound of Formula (I) (preferably, the compound of Formula (I-g)) and abiraterone acetate in the manufacture of a medicament for the treatment and/or prevention of prostate cancer.

As referred to herein, a compound of Formula (I) refers to a compound with the following structure:

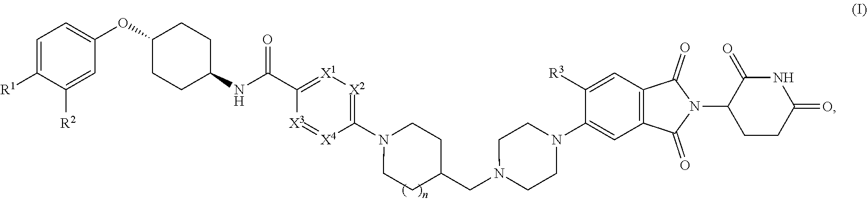

or a pharmaceutically acceptable salt, enantiomer, stereoisomer, solvate, polymorph, isotopic derivative, or prodrug thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1.

In some embodiments, a compound of Formula (I) refers to a compound with the following structure:

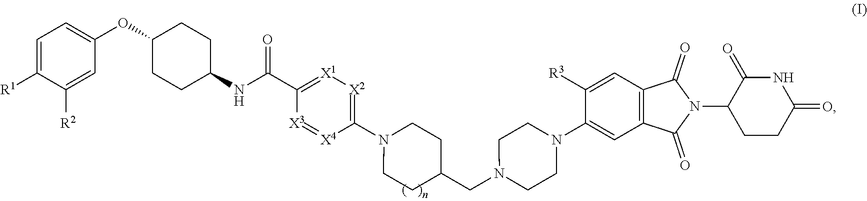

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1.

In some embodiments, a compound of Formula (I) refers to a compound with the following structure:

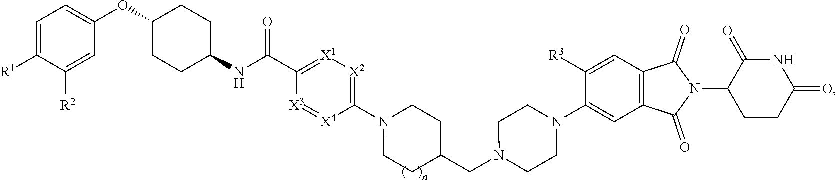

wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1.

In some embodiments, R¹ is hydrogen.

In some embodiments, R¹ is CN.

In some embodiments, R¹ is C₁-C₆ alkyl.

In some embodiments, R² is hydrogen.

In some embodiments, R² is halo. In some embodiments, R² is F. In some embodiments, R² is Cl. In some embodiments, R² is Br. In some embodiments, R² is I.

In some embodiments, R² is C₁-C₆ alkyl.

In some embodiments, R³ is hydrogen.

In some embodiments, R³ is halo. In some embodiments, R³ is F. In some embodiments, R³ is Cl. In some embodiments, R³ is Br. In some embodiments, R³ is I.

In some embodiments, at least one of X¹, X², X³, and X⁴ is CH.

In some embodiments, at least two of X¹, X², X³, and X⁴ are CH.

In some embodiments, at least three of X¹, X², X³, and X⁴ are CH.

In some embodiments, each of X¹, X², X³, and X⁴ is CH.

In some embodiments, X¹, X², and X³ are each CH, and X⁴ is N.

In some embodiments, X¹, X², and X⁴ are each CH, and X³ is N.

In some embodiments, X¹, X³, and X⁴ are each CH, and X² is N.

In some embodiments, X², X³, and X⁴ are each CH, and X¹ is N.

In some embodiments, X¹ and X² are each CH, and X³ and X⁴ are each N.

In some embodiments, X¹ and X³ are each CH, and X² and X⁴ are each N.

In some embodiments, X¹ and X⁴ are each CH, and X² and X³ are each N.

In some embodiments, X² and X³ are each CH, and X¹ and X⁴ are each N.

In some embodiments, X² and X⁴ are each CH, and X¹ and X³ are each N.

In some embodiments, X³ and X⁴ are each CH, and X¹ and X² are each N.

In some embodiments, n is 0.

In some embodiments, n is 1.

In some embodiments, the compound of Formula (I) is

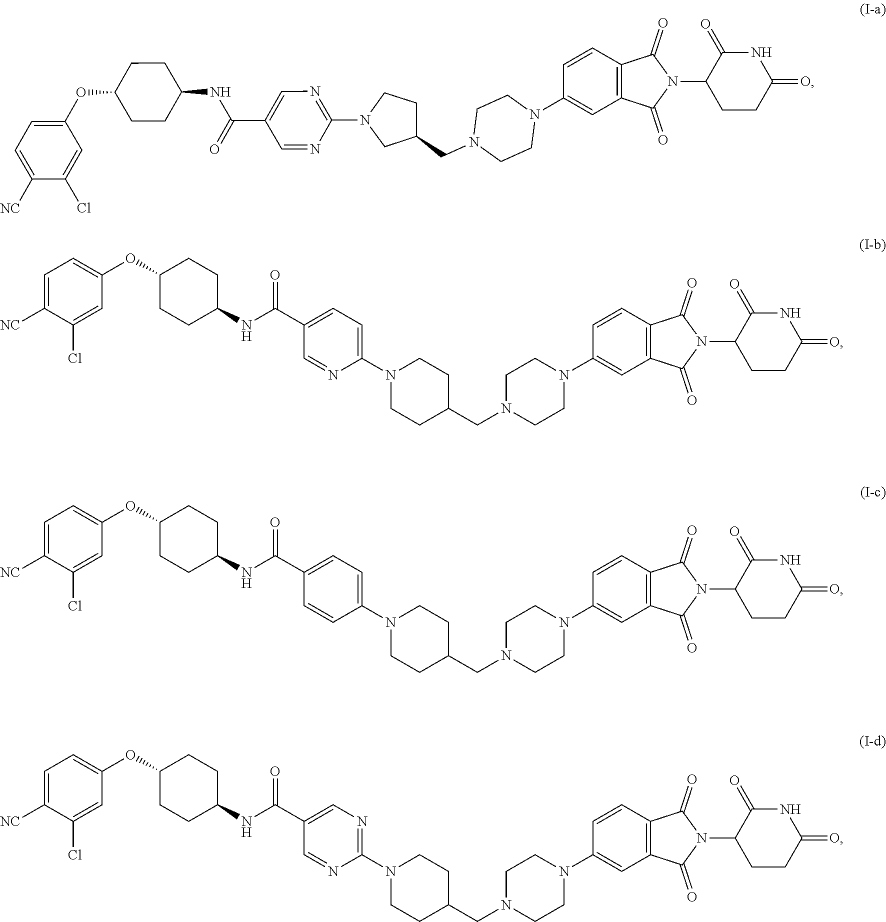 [SMILES: N#Cc1ccc(O[C@H]2CC[C@H](NC(=O)c3cnc(N4CC[C@@H](CN5CCN(c6ccc7c(c6)C(=O)N(C6CCC(=O)NC6=O)C7=O)CC5)C4)nc3)CC2)cc1Cl ; N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nc3)CC2)cc1Cl ; N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)cc3)CC2)cc1Cl ; N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)nc3)CC2)cc1Cl]

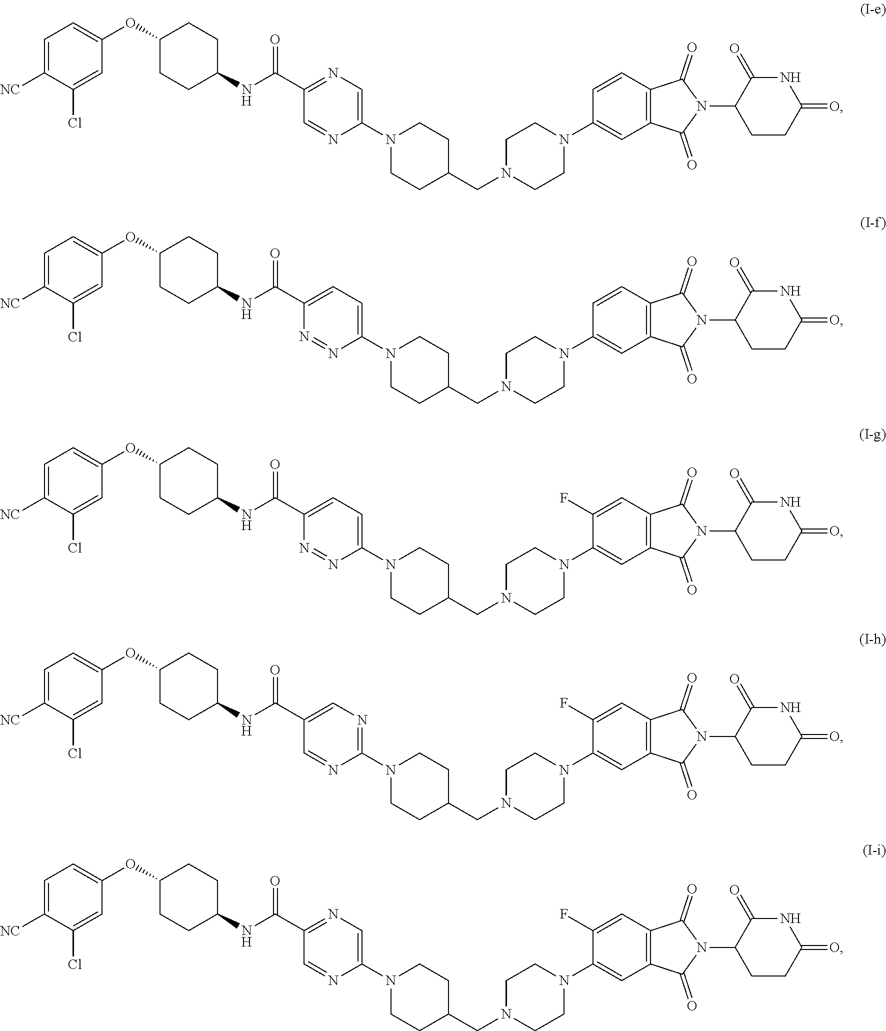

or a pharmaceutically acceptable salt thereof.

In some embodiments, the compound of Formula (I) is the compound of Formula (I-a):

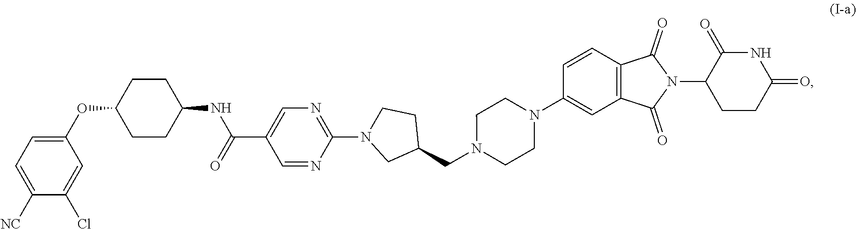 [SMILES: N#Cc1ccc(O[C@H]2CC[C@H](NC(=O)c3cnc(N4CC[C@@H](CN5CCN(c6ccc7c(c6)C(=O)N(C6CCC(=O)NC6=O)C7=O)CC5)C4)nc3)CC2)cc1Cl]

or a pharmaceutically acceptable salt thereof.

In some embodiments, the compound of Formula (I) is the compound of Formula (I-b):

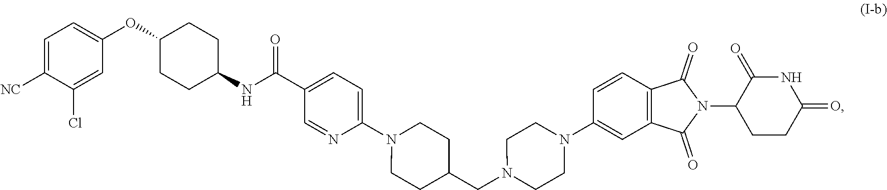 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nc3)CC2)cc1Cl]

or a pharmaceutically acceptable salt thereof.

In some embodiments, the compound of Formula (I) is the compound of Formula (I-c):

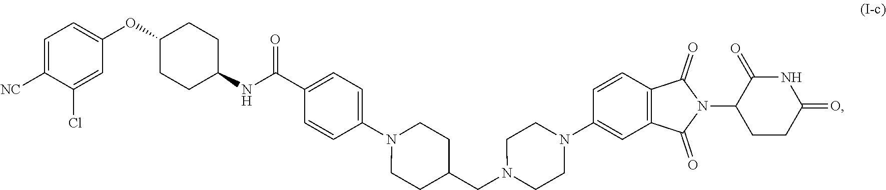 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)cc3)CC2)cc1Cl]

or a pharmaceutically acceptable salt thereof.

In some embodiments, the compound of Formula (I) is the compound of Formula (I-d):

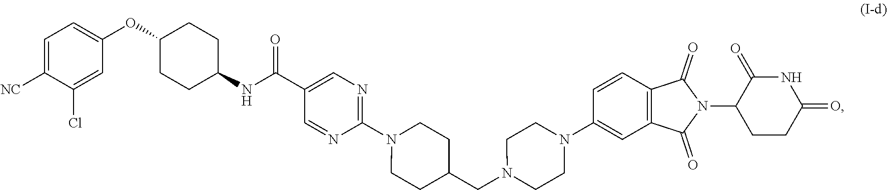 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)nc3)CC2)cc1Cl]

or a pharmaceutically acceptable salt thereof.

In some embodiments, the compound of Formula (I) is the compound of Formula (I-e):

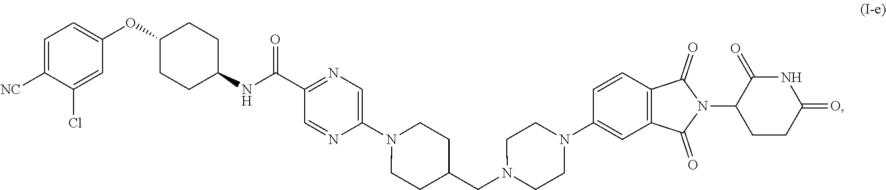 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)cn3)CC2)cc1Cl]

or a pharmaceutically acceptable salt thereof.

In some embodiments, the compound of Formula (I) is the compound of Formula (I-f):

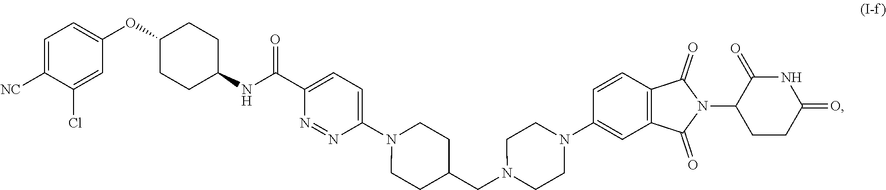 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nn3)CC2)cc1Cl]

or a pharmaceutically acceptable salt thereof.

In some embodiments, the compound of Formula (I) is the compound of Formula (I-g):

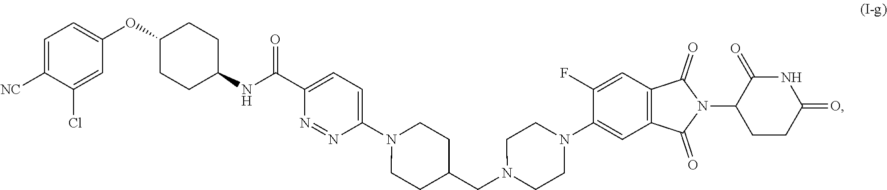 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof.

In some embodiments, the compound of Formula (I) is the compound of Formula (I-g), or “Compound (I-g)”:

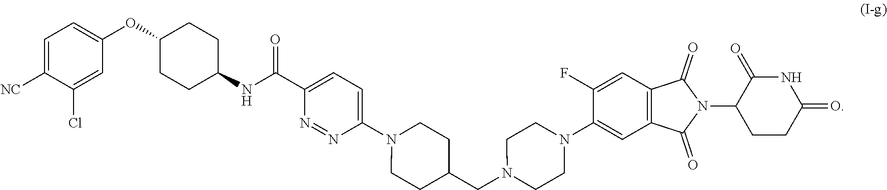 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

In some embodiments, the compound of Formula (I) is the compound of Formula (I-h):

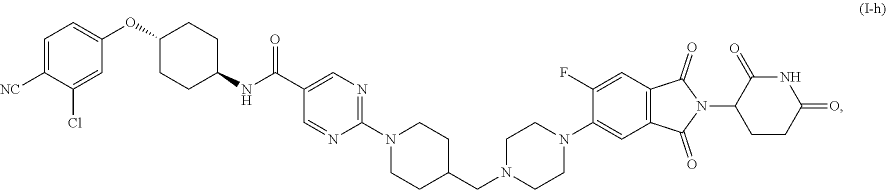 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl]

or a pharmaceutically acceptable salt thereof.

In some embodiments, the compound of Formula (I) is the compound of Formula (I-i):

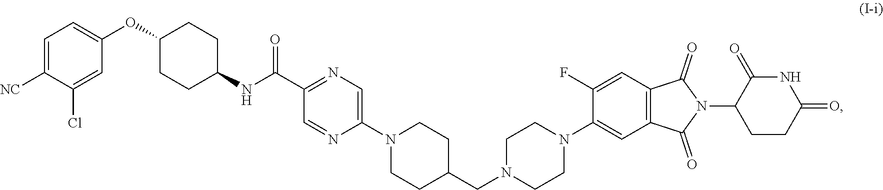 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt thereof.

A compound of Formula (I) may be synthesized using standard synthetic methods and procedures for the preparation of organic molecules and functional group transformations and manipulations, including the use of protective groups, as can be obtained from the relevant scientific literature or from standard reference textbooks in the field. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}; John Wiley & Sons: New York, 1999. Methods for preparing a compound of Formula (I) are described in U.S. Patent Application Publication No. 2023/0012321 and U.S. Pat. No. 10,584,101, the contents of which are incorporated herein in their entirety.

### Methods of Ubiquitinating/Degrading a Target Protein in a Cell

The present invention provides a method of ubiquitinating/degrading a target protein in a cell. The method comprises administering a bifunctional composition comprising an E3 ubiquitin ligase binding moiety and a protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., an ubiquitin ligase, preferably an E3 ubiquitin ligase) and the protein targeting moiety recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cells of a patient.

In some embodiments, the present invention is directed to a method of treating a patient in need for a disease state or condition modulated through a protein where the degradation of that protein will produce a therapeutic effect in that patient, the method comprising administering to a patient in need an effective amount of a compound according to the present invention (e.g., Compound (I-g)) optionally in combination with another bioactive agent (e.g., abiraterone or abiraterone acetate). The disease state or condition may be a disease caused by a microbial agent or other exogenous agent such as a virus, bacteria, fungus, protozoa or other microbe or may be a disease state, which is caused by overexpression of a protein, which leads to a disease state and/or condition.

### Methods of Treatment

In one aspect, the present application pertains to a method of treating and/or preventing cancer comprising administering to a subject in need thereof a therapeutically effective amount of a compound of Formula (I), or a pharmaceutically acceptable salt thereof.

In one aspect, the present application pertains to a method of treating and/or preventing prostate cancer comprising administering to a subject in need thereof a therapeutically effective amount of a compound of Formula (I), or a pharmaceutically acceptable salt thereof.

In one aspect, the present application pertains to a method of treating and/or preventing prostate cancer comprising administering to a subject in need thereof a therapeutically effective amount of a compound of Formula (I), or a pharmaceutically acceptable salt thereof, and abiraterone.

In one aspect, the present application pertains to a method of treating and/or preventing prostate cancer comprising administering to a subject in need thereof a therapeutically effective amount of a compound of Formula (I), or a pharmaceutically acceptable salt thereof, and abiraterone acetate.

In one aspect, the present application pertains to a method of treating and/or preventing prostate cancer comprising administering to a subject in need thereof a therapeutically effective amount of a compound of Formula (I-g), or a pharmaceutically acceptable salt thereof, and abiraterone.

In one aspect, the present application pertains to a method of treating and/or preventing prostate cancer comprising administering to a subject in need thereof a therapeutically effective amount of a compound of Formula (I-g), or a pharmaceutically acceptable salt thereof, and abiraterone acetate.

In one aspect, the present application pertains to a method of treating and/or preventing prostate cancer comprising administering to a subject in need thereof a therapeutically effective amount of a compound of Formula (I-g) and abiraterone.

In one aspect, the present application pertains to a method of treating and/or preventing prostate cancer comprising administering to a subject in need thereof a therapeutically effective amount of a compound of Formula (I-g) and abiraterone acetate.

In one aspect, the present application pertains to a method of treating prostate cancer comprising administering to a subject in need thereof a therapeutically effective amount of a compound of Formula (I-g) and abiraterone.

In one aspect, the present application pertains to a method of treating prostate cancer comprising administering to a subject in need thereof a therapeutically effective amount of a compound of Formula (I-g) and abiraterone acetate.

The methods of treating cancer described herein include a reduction in tumor size. Alternatively, or in addition, the cancer is metastatic cancer and this method of treatment includes inhibition of metastatic cancer cell invasion.

In some embodiments, the cancer is prostate cancer.

In some embodiments, the cancer is adenocarcinoma of the prostate.

In some embodiments, the cancer is metastatic prostate cancer.

In some embodiments, the cancer is castrate-resistant prostate cancer.

In some embodiments, the cancer is metastatic castrate-resistant prostate cancer (mCRPC).

In some embodiments, the cancer is progressive mCRPC.

In some embodiments, the prostate cancer is castrate-sensitive prostate cancer.

In some embodiments, the prostate cancer is metastatic castrate-sensitive prostate cancer.

In some embodiments, the prostate cancer is prostate cancer naïve to novel hormonal agents (NHA).

In some embodiments, the prostate cancer is metastatic prostate cancer naïve to novel hormonal agents (NHA).

In some embodiments, the prostate cancer is castrate-resistant prostate cancer naïve to novel hormonal agents (NHA).

In some embodiments, the prostate cancer is castrate-sensitive prostate cancer naïve to novel hormonal agents (NHA).

In some embodiments, the prostate cancer is metastatic castrate-resistant prostate cancer naïve to novel hormonal agents (NHA).

In some embodiments, the prostate cancer is metastatic castrate-sensitive prostate cancer naïve to novel hormonal agents (NHA).

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 1000 mg of the compound of Formula (I-g):

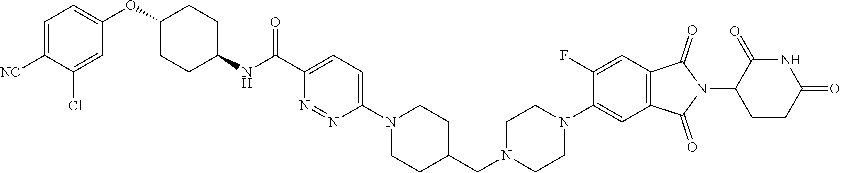 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 1000 mg of the compound of Formula (I-g):

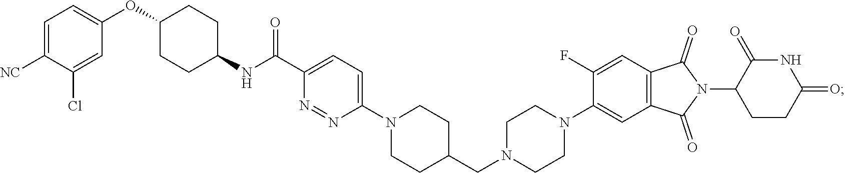 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 1000 mg of the compound of Formula (I-g):

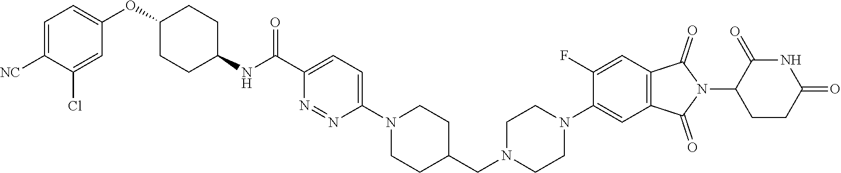 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 1000 mg of the compound of Formula (I-g):

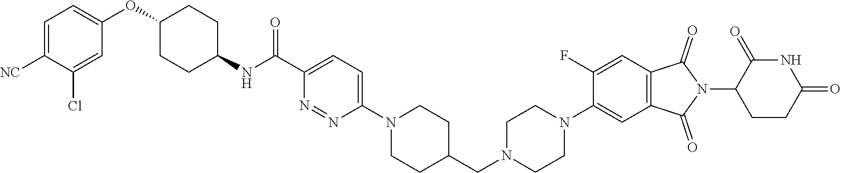 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 850 mg of the compound of Formula

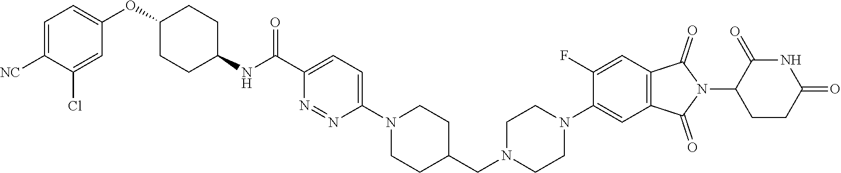 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 850 mg of the compound of Formula

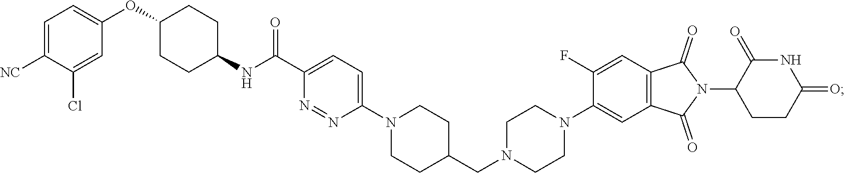 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 850 mg of the compound of Formula (I-g):

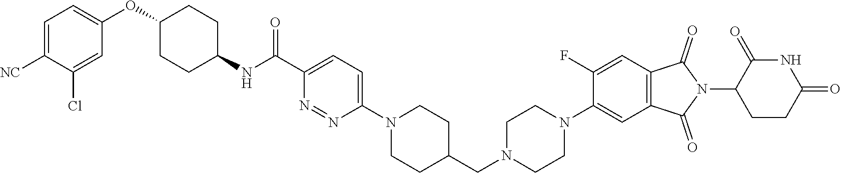 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 850 mg of the compound of Formula (I-g):

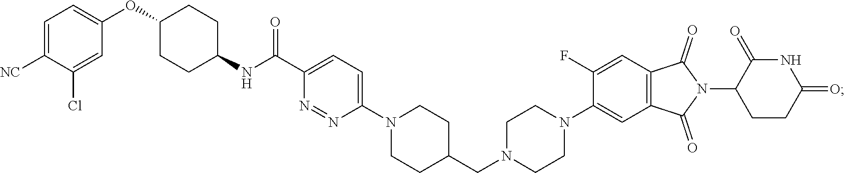 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 700 mg of the compound of Formula (I-g):

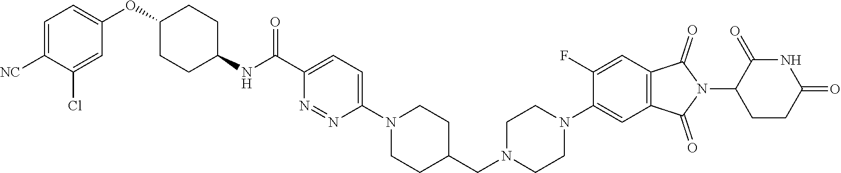 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 700 mg of the compound of Formula

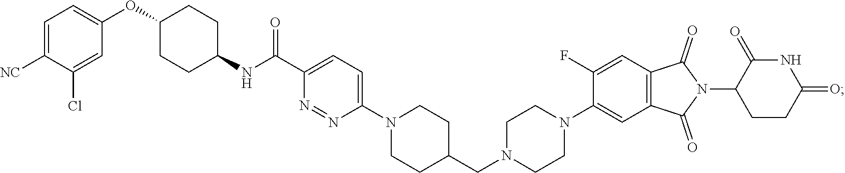 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 700 mg of the compound of Formula (I-g):

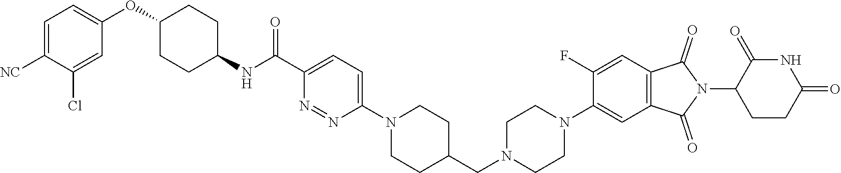 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 700 mg of the compound of Formula (I-g):

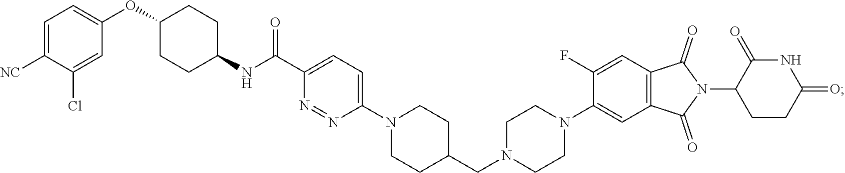 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 250 mg to about 500 mg of the compound of Formula (I-g):

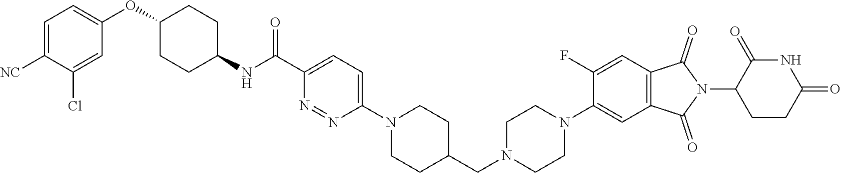 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 250 mg to about 500 mg of the compound of Formula (I-g):

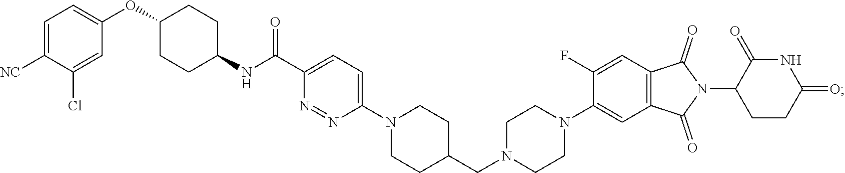 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 250 mg to about 500 mg of the compound of Formula

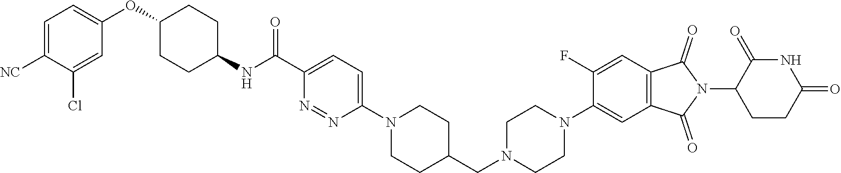 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 250 mg to about 500 mg of the compound of Formula (I-g):

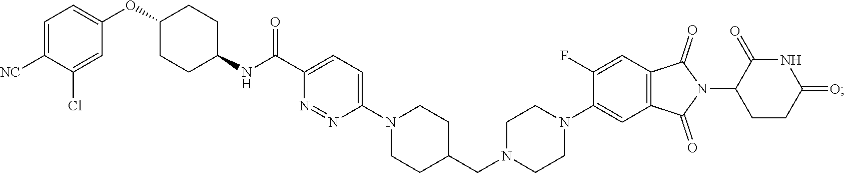 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 350 mg of the compound of Formula (I-g):

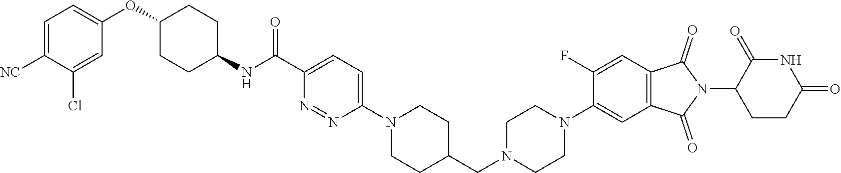 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, once daily administration of about 35 mg to about 350 mg of the compound of Formula (I-g):

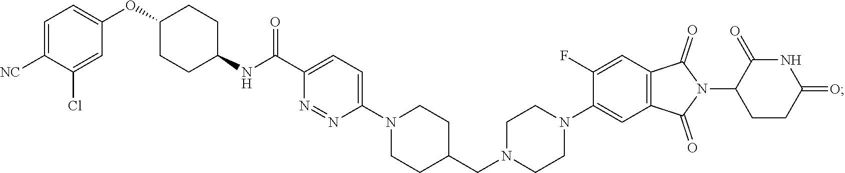 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 350 mg of the compound of Formula (I-g):

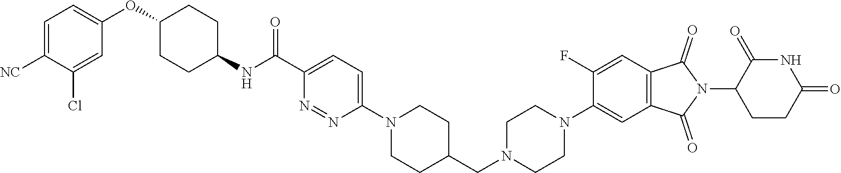 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA) in a subject in need thereof, comprising:
(i) oral, twice daily administration of about 35 mg to about 350 mg of the compound of Formula

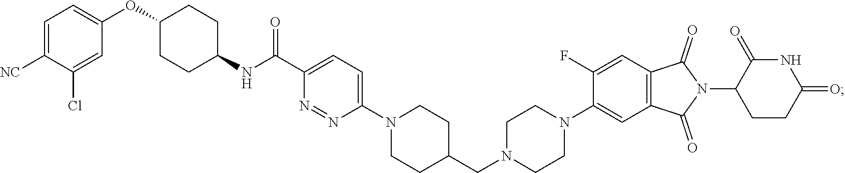 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate, or a pharmaceutically acceptable salt thereof.

In one aspect, the present application pertains to a method of treating prostate cancer in a subject in need thereof, comprising:
(i) oral, once daily administration of about 35 mg to about 500 mg of the compound of Formula (J-g):

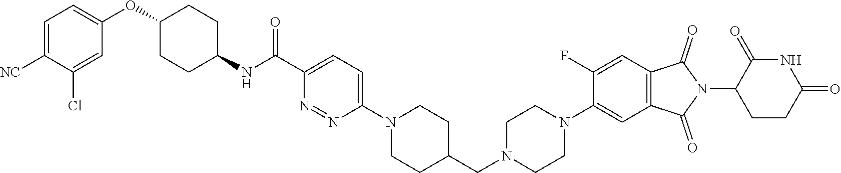 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating prostate cancer in a subject in need thereof, comprising:
(i) oral, once daily administration of about 35 mg to about 500 mg of the compound of Formula (I-g):

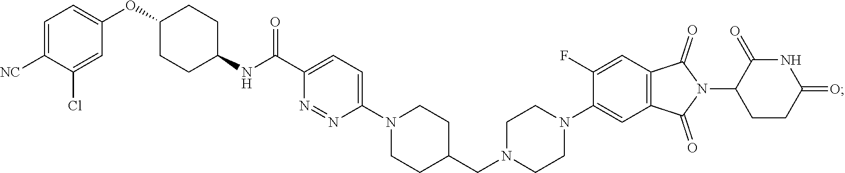 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating prostate cancer in a subject in need thereof, comprising:
(i) oral, once daily administration of about 35 mg to about 500 mg of the compound of Formula

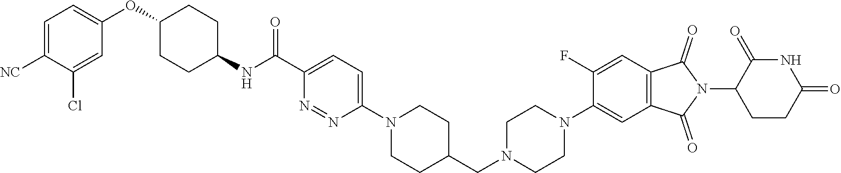 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating prostate cancer in a subject in need thereof, comprising:
(i) oral, once daily administration of about 35 mg to about 500 mg of the compound of Formula (I-g):

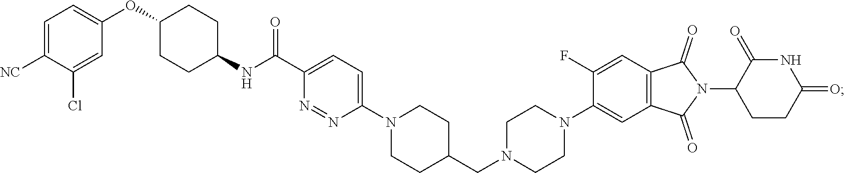 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating adenocarcinoma of the prostate in a subject in need thereof, comprising:
(i) oral, once daily administration of about 35 mg to about 500 mg of the compound of Formula (I-g):

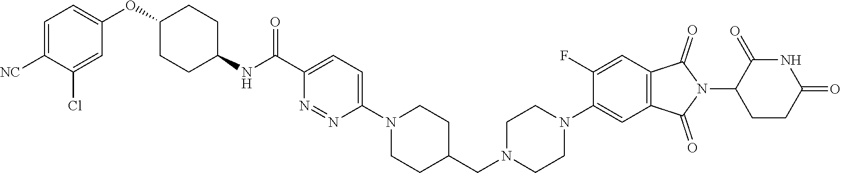 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating adenocarcinoma of the prostate in a subject in need thereof, comprising:
(i) oral, once daily administration of about 35 mg to about 500 mg of the compound of Formula (I-g):

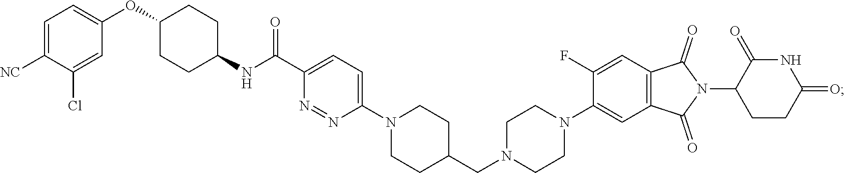 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating adenocarcinoma of the prostate in a subject in need thereof, comprising:
(i) oral, once daily administration of about 35 mg to about 500 mg of the compound of Formula (I-g):

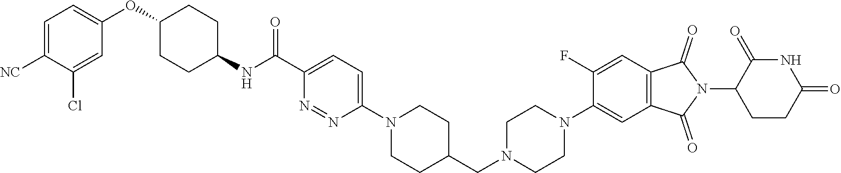 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating adenocarcinoma of the prostate in a subject in need thereof, comprising:
(i) oral, once daily administration of about 35 mg to about 500 mg of the compound of Formula (I-g):

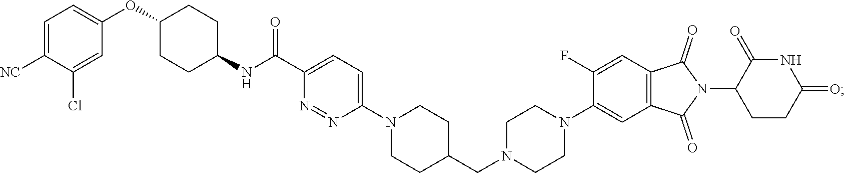 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic prostate cancer in a subject in need thereof, comprising:
(i) oral, once daily administration of about 35 mg to about 500 mg of the compound of Formula (I-g):

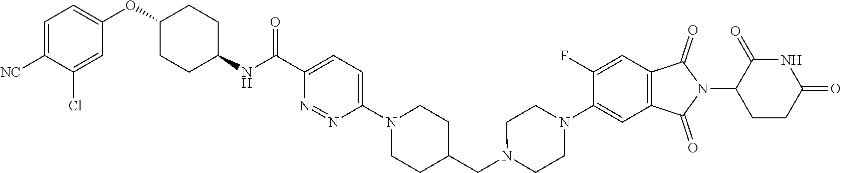 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic prostate cancer in a subject in need thereof, comprising:
(i) oral, once daily administration of about 35 mg to about 500 mg of the compound of Formula (I-g):

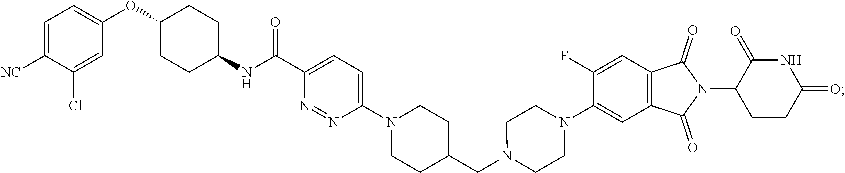 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic prostate cancer in a subject in need thereof, comprising:
(i) oral, once daily administration of about 35 mg to about 500 mg of the compound of Formula

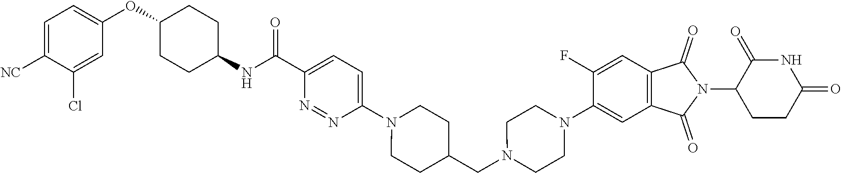 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer in a subject in need thereof, comprising:
(i) oral, once daily administration of about 35 mg to about 500 mg of the compound of Formula (I-g):

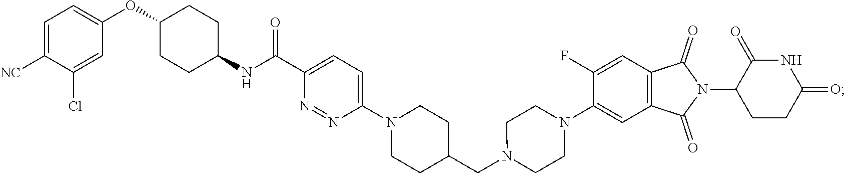 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-resistant prostate cancer in a subject in need thereof, comprising:
(i) oral, once daily administration of about 35 mg to about 500 mg of the compound of Formula (I-g):

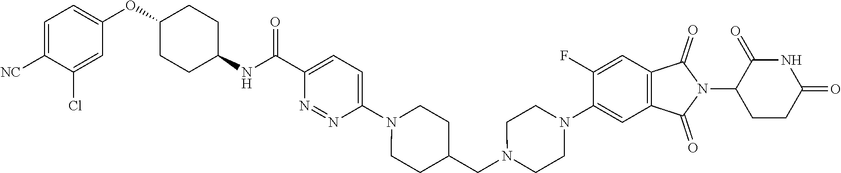 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating castrate-resistant prostate cancer in a subject in need thereof, comprising:
(i) oral, once daily administration of about 35 mg to about 500 mg of the compound of Formula (I-g):

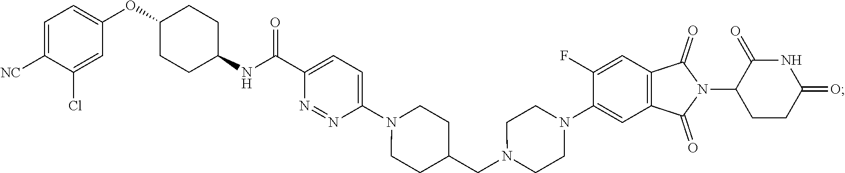 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating castrate-resistant prostate cancer in a subject in need thereof, comprising:
(i) oral, once daily administration of about 35 mg to about 500 mg of the compound of Formula (I-g):

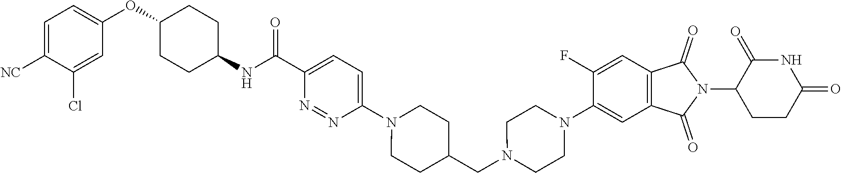 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating castrate-resistant prostate cancer in a subject in need thereof, comprising:
(i) oral, once daily administration of about 35 mg to about 500 mg of the compound of Formula (I-g):

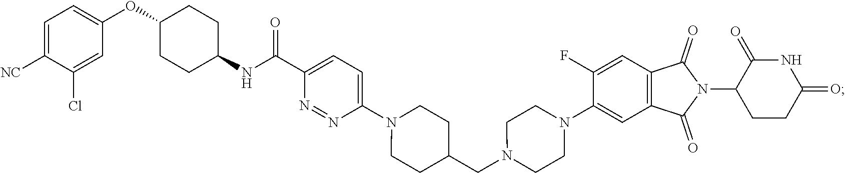 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-resistant prostate cancer in a subject in need thereof, comprising:
(i) oral, once daily administration of about 35 mg to about 500 mg of the compound of Formula (I-g):

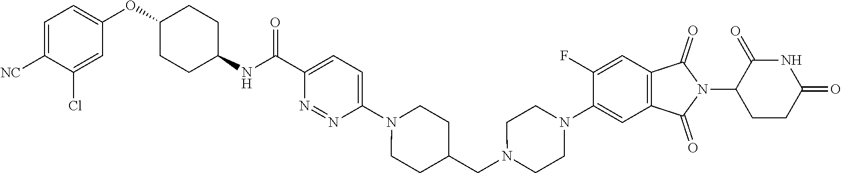 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-resistant prostate cancer in a subject in need thereof, comprising:
(i) oral, once daily administration of about 35 mg to about 500 mg of the compound of Formula (I-g):

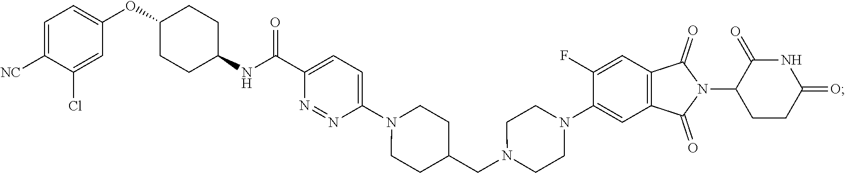 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-resistant prostate cancer in a subject in need thereof, comprising:
(i) oral, once daily administration of about 35 mg to about 500 mg of the compound of Formula (I-g):

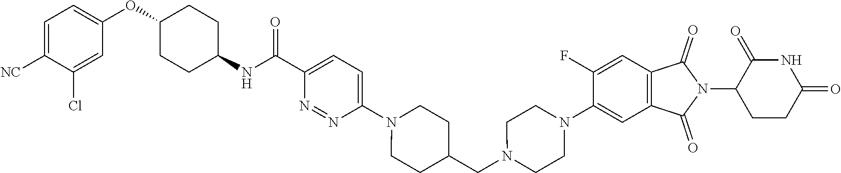 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-resistant prostate cancer in a subject in need thereof, comprising:
(i) oral, once daily administration of about 35 mg to about 500 mg of the compound of Formula (I-g):

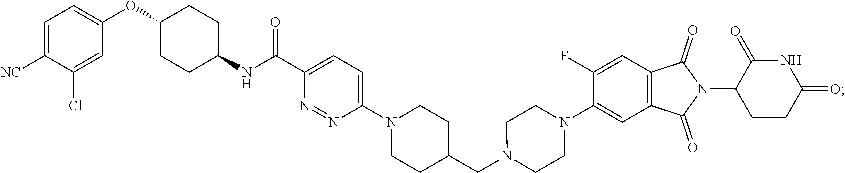 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating progressive mCRPC in a subject in need thereof, comprising:
(i) oral, once daily administration of about 35 mg to about 500 mg of the compound of Formula (I-g):

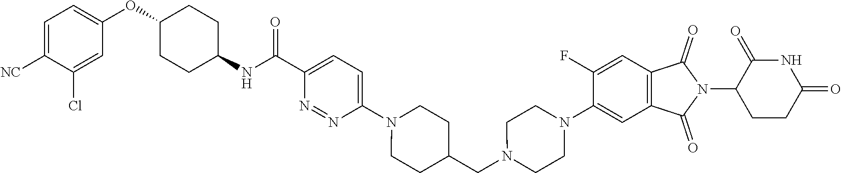 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating progressive mCRPC in a subject in need thereof, comprising:
(i) oral, once daily administration of about 35 mg to about 500 mg of the compound of Formula (I-g):

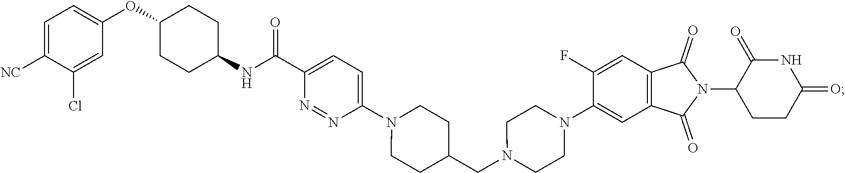 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating progressive mCRPC in a subject in need thereof, comprising:
(i) oral, once daily administration of about 35 mg to about 500 mg of the compound of Formula (I-g):

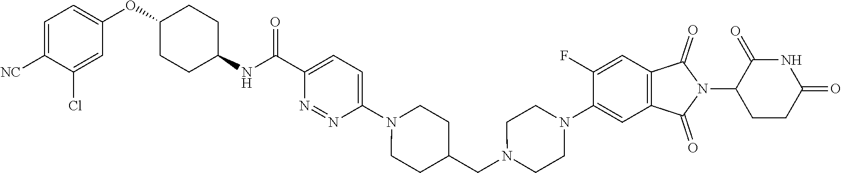 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

(ii) oral, once daily administration of about 1000 mg abiraterone acetate, or a pharmaceutically acceptable salt thereof.

In one aspect, the present application pertains to a method of treating progressive mCRPC in a subject in need thereof, comprising:
(i) oral, once daily administration of about 35 mg to about 500 mg of the compound of Formula (I-g):

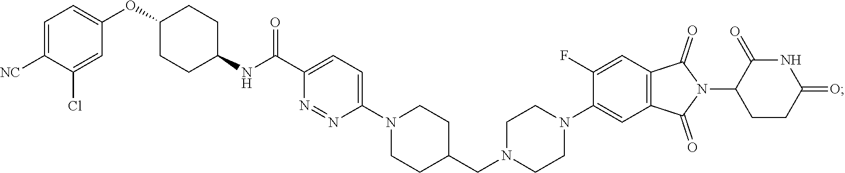 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-sensitive prostate cancer in a subject in need thereof, comprising:
(i) oral, once daily administration of about 35 mg to about 500 mg of the compound of Formula (I-g):

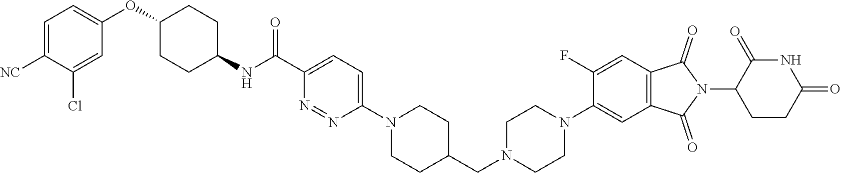 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating castrate-sensitive prostate cancer in a subject in need thereof, comprising:
(i) oral, once daily administration of about 35 mg to about 500 mg of the compound of Formula

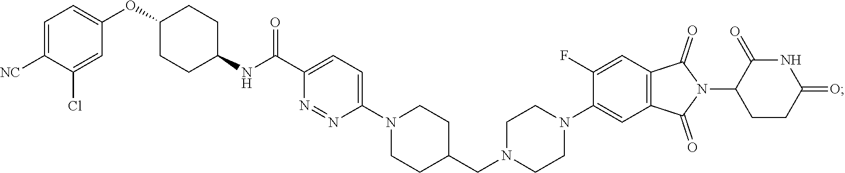 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating castrate-sensitive prostate cancer in a subject in need thereof, comprising:
(i) oral, once daily administration of about 35 mg to about 500 mg of the compound of Formula (I-g):

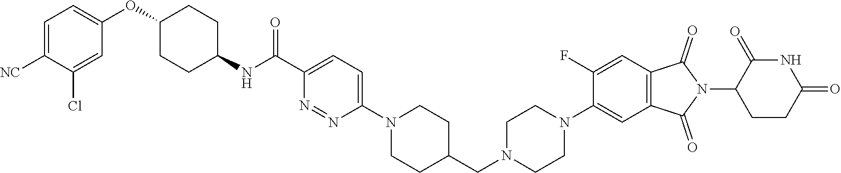 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating castrate-sensitive prostate cancer in a subject in need thereof, comprising:
(i) oral, once daily administration of about 35 mg to about 500 mg of the compound of Formula (I-g):

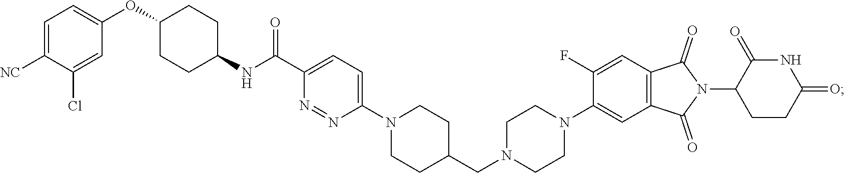 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-sensitive prostate cancer in a subject in need thereof, comprising:
(i) oral, once daily administration of about 35 mg to about 500 mg of the compound of Formula

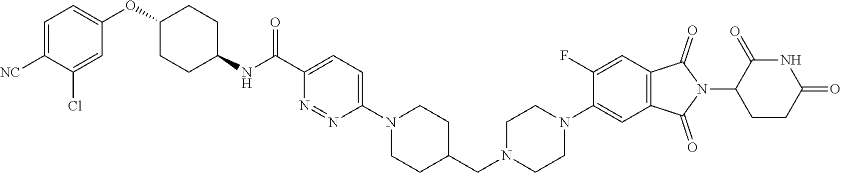 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-sensitive prostate cancer in a subject in need thereof, comprising:
(i) oral, once daily administration of about 35 mg to about 500 mg of the compound of Formula (I-g):

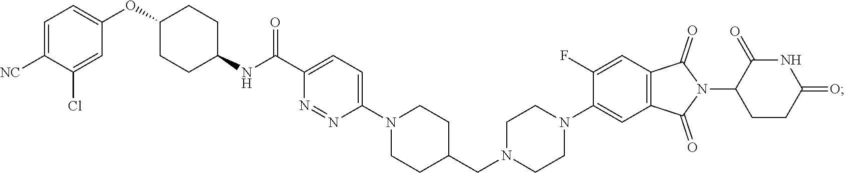 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-sensitive prostate cancer in a subject in need thereof, comprising:
(i) oral, once daily administration of about 35 mg to about 500 mg of the compound of Formula (I-g):

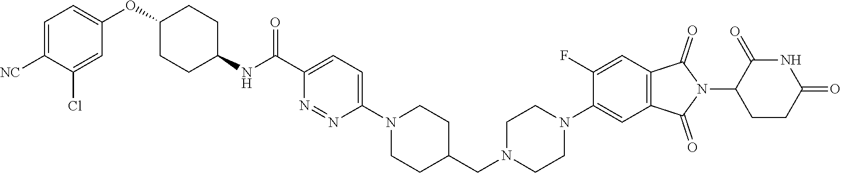 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-sensitive prostate cancer in a subject in need thereof, comprising:
(i) oral, once daily administration of about 35 mg to about 500 mg of the compound of Formula (I-g):

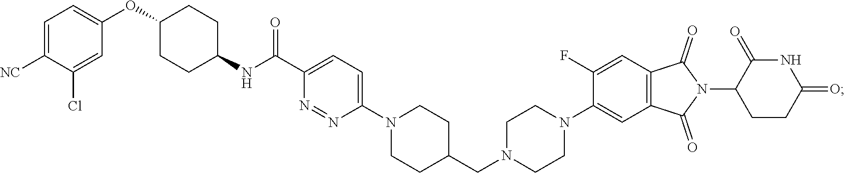 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating prostate cancer naïve to novel hormonal agents (NHA) in a subject in need thereof, comprising:
(i) oral, once daily administration of about 35 mg to about 500 mg of the compound of Formula (I-g):

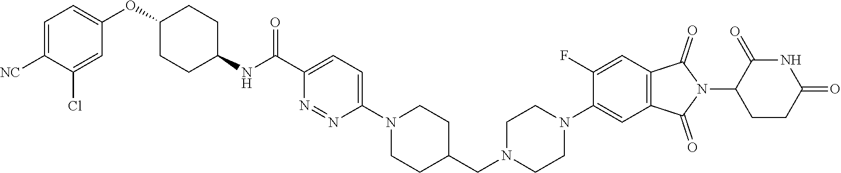 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating prostate cancer naïve to novel hormonal agents (NHA) in a subject in need thereof, comprising:
(i) oral, once daily administration of about 35 mg to about 500 mg of the compound of Formula (I-g):

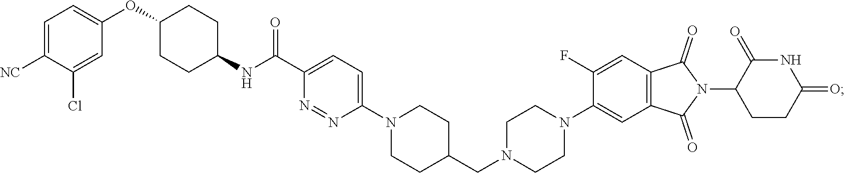 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating prostate cancer naïve to novel hormonal agents (NHA) in a subject in need thereof, comprising:
(i) oral, once daily administration of about 35 mg to about 500 mg of the compound of Formula (I-g):

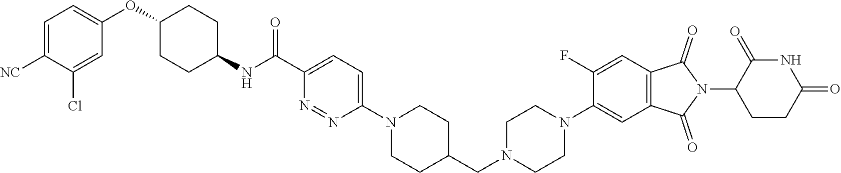 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating prostate cancer naïve to novel hormonal agents (NHA) in a subject in need thereof, comprising:
(i) oral, once daily administration of about 35 mg to about 500 mg of the compound of Formula (I-g):

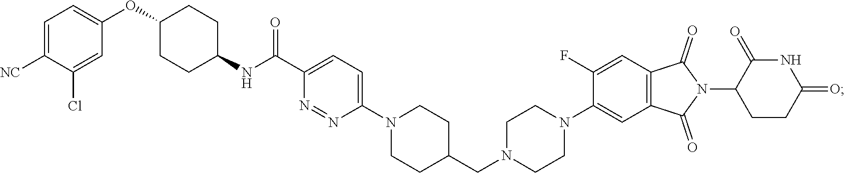 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic prostate cancer naïve to novel hormonal agents (NHA) in a subject in need thereof, comprising:
(i) oral, once daily administration of about 35 mg to about 500 mg of the compound of Formula (I-g):

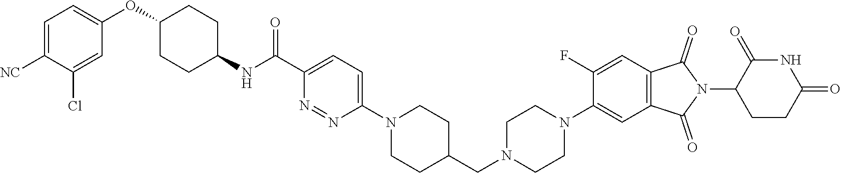 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic prostate cancer naïve to novel hormonal agents (NHA) in a subject in need thereof, comprising:
(i) oral, once daily administration of about 35 mg to about 500 mg of the compound of Formula (I-g):

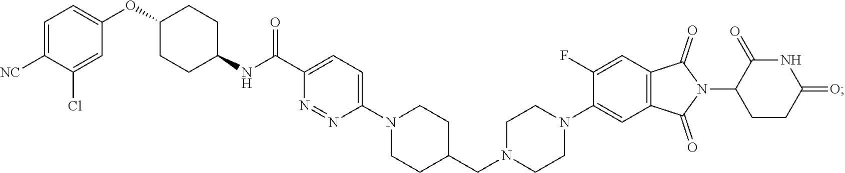 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic prostate cancer naïve to novel hormonal agents (NHA) in a subject in need thereof, comprising:
(i) oral, once daily administration of about 35 mg to about 500 mg of the compound of Formula (I-g):

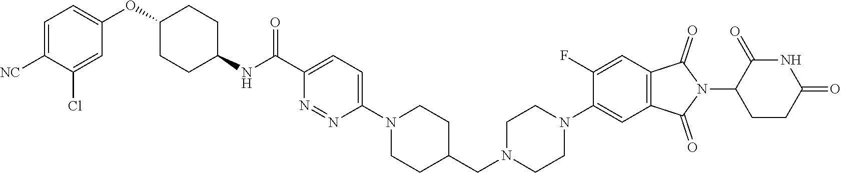 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer naïve to novel hormonal agents (NHA) in a subject in need thereof, comprising:
(i) oral, once daily administration of about 35 mg to about 500 mg of the compound of Formula (I-g):

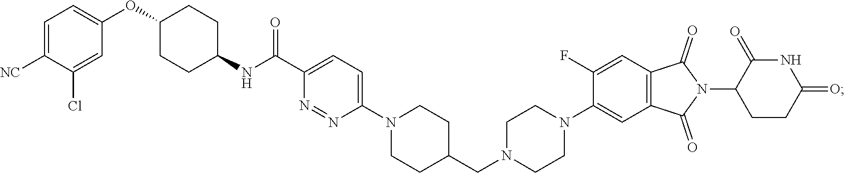 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-resistant prostate cancer naïve to novel hormonal agents (NHA) in a subject in need thereof, comprising:
(i) oral, once daily administration of about 35 mg to about 500 mg of the compound of Formula (I-g):

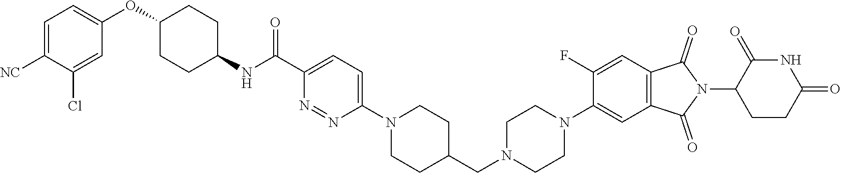 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating castrate-resistant prostate cancer naïve to novel hormonal agents (NHA) in a subject in need thereof, comprising:
(i) oral, once daily administration of about 35 mg to about 500 mg of the compound of Formula (I-g):

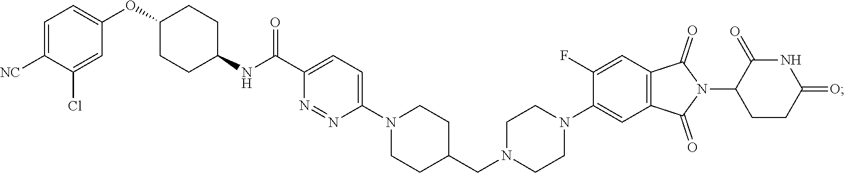 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating castrate-resistant prostate cancer naïve to novel hormonal agents (NHA) in a subject in need thereof, comprising:
(i) oral, once daily administration of about 35 mg to about 500 mg of the compound of Formula (I-g):

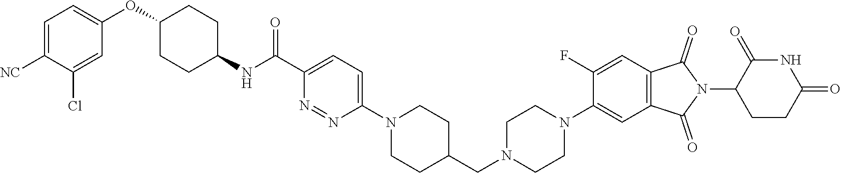 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

(ii) oral, once daily administration of about 1000 mg abiraterone acetate, or a pharmaceutically acceptable salt thereof.

In one aspect, the present application pertains to a method of treating castrate-resistant prostate cancer naïve to novel hormonal agents (NHA) in a subject in need thereof, comprising:
(i) oral, once daily administration of about 35 mg to about 500 mg of the compound of Formula (I-g):

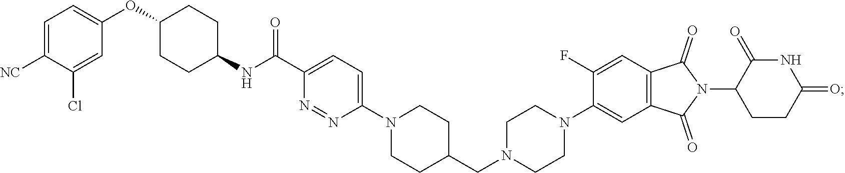 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-sensitive prostate cancer naïve to novel hormonal agents (NHA) in a subject in need thereof, comprising:
(i) oral, once daily administration of about 35 mg to about 500 mg of the compound of Formula (I-g):

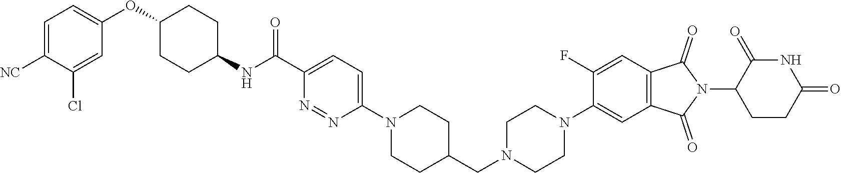 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating castrate-sensitive prostate cancer naïve to novel hormonal agents (NHA) in a subject in need thereof, comprising:
(i) oral, once daily administration of about 35 mg to about 500 mg of the compound of Formula (I-g):

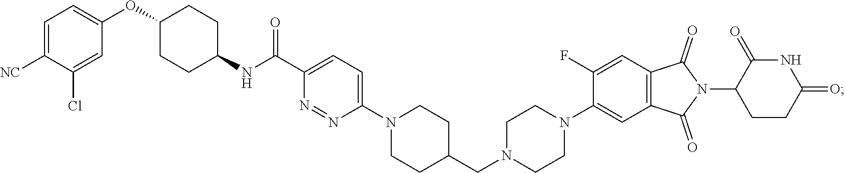 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating castrate-sensitive prostate cancer naïve to novel hormonal agents (NHA) in a subject in need thereof, comprising:
(i) oral, once daily administration of about 35 mg to about 500 mg of the compound of Formula (I-g):

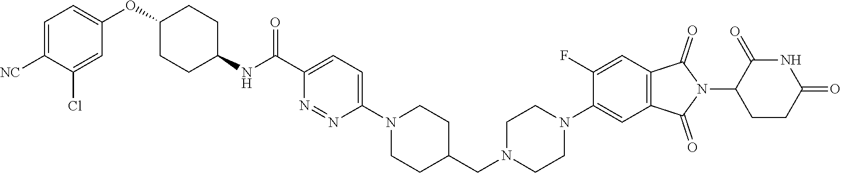 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating castrate-sensitive prostate cancer naïve to novel hormonal agents (NHA) in a subject in need thereof, comprising:
(i) oral, once daily administration of about 35 mg to about 500 mg of the compound of Formula (I-g):

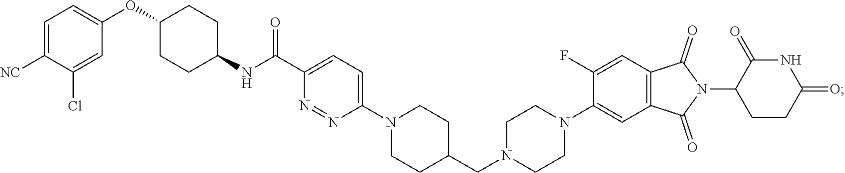 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-resistant prostate cancer naïve to novel hormonal agents (NHA) in a subject in need thereof, comprising:
(i) oral, once daily administration of about 35 mg to about 500 mg of the compound of Formula

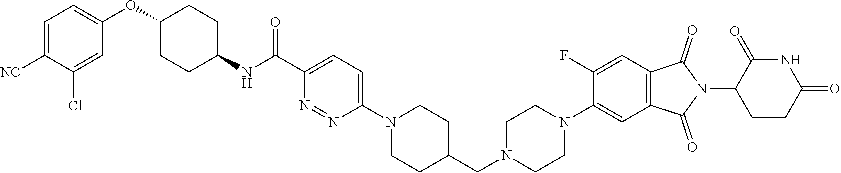 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-resistant prostate cancer naïve to novel hormonal agents (NHA) in a subject in need thereof, comprising:
(i) oral, once daily administration of about 35 mg to about 500 mg of the compound of Formula (I-g):

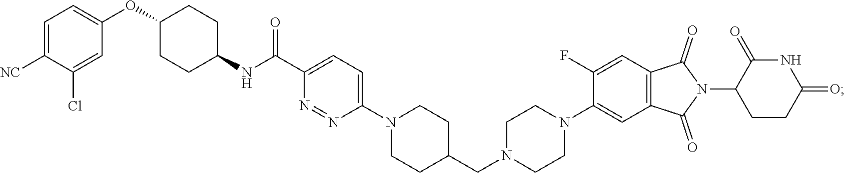 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-resistant prostate cancer naïve to novel hormonal agents (NHA) in a subject in need thereof, comprising:
(i) oral, once daily administration of about 35 mg to about 500 mg of the compound of Formula (I-g):

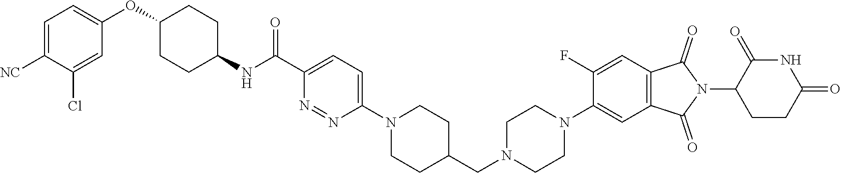 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-resistant prostate cancer naïve to novel hormonal agents (NHA) in a subject in need thereof, comprising:
(i) oral, once daily administration of about 35 mg to about 500 mg of the compound of Formula (I-g):

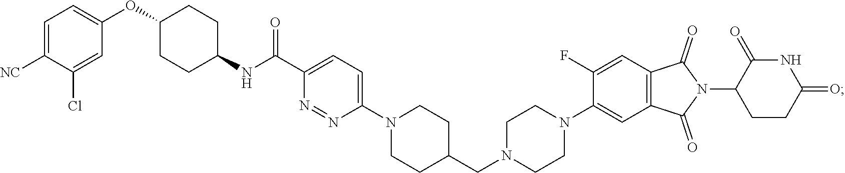 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-sensitive prostate cancer naïve to novel hormonal agents (NHA) in a subject in need thereof, comprising:
(i) oral, once daily administration of about 35 mg to about 500 mg of the compound of Formula (I-g):

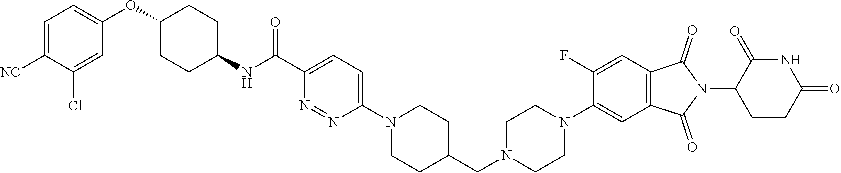 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-sensitive prostate cancer naïve to novel hormonal agents (NHA) in a subject in need thereof, comprising:
(i) oral, once daily administration of about 35 mg to about 500 mg of the compound of Formula

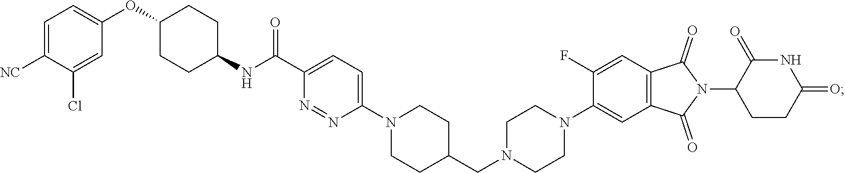 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-sensitive prostate cancer naïve to novel hormonal agents (NHA) in a subject in need thereof, comprising:
(i) oral, once daily administration of about 35 mg to about 500 mg of the compound of Formula (I-g):

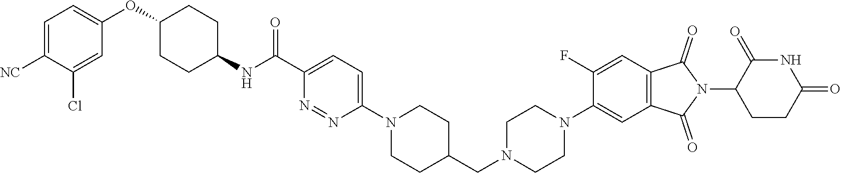 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-sensitive prostate cancer naïve to novel hormonal agents (NHA) in a subject in need thereof, comprising:
(i) oral, once daily administration of about 35 mg to about 500 mg of the compound of Formula (I-g):

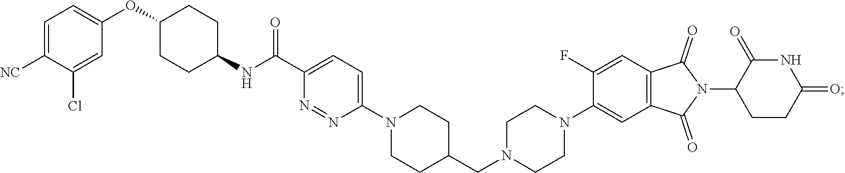 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating prostate cancer in a subject in need thereof, comprising:
(i) oral, once daily administration of about 400 mg to about 500 mg of the compound of Formula (I-g):

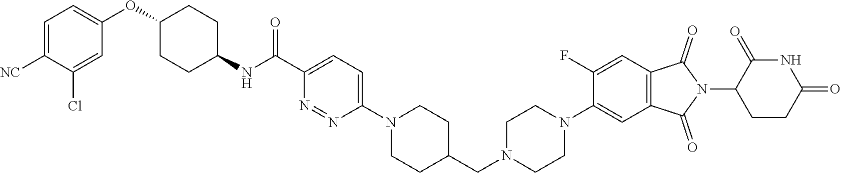 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating prostate cancer in a subject in need thereof, comprising:
(i) oral, once daily administration of about 400 mg to about 500 mg of the compound of Formula (I-g):

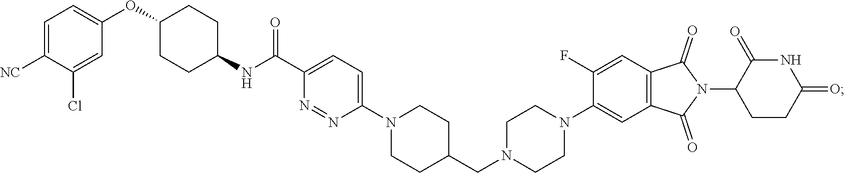 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating prostate cancer in a subject in need thereof, comprising:
(i) oral, once daily administration of about 400 mg to about 500 mg of the compound of Formula (I-g):

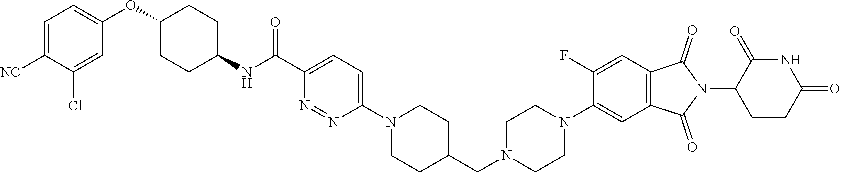 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating prostate cancer in a subject in need thereof, comprising:
(i) oral, once daily administration of about 400 mg to about 500 mg of the compound of Formula (I-g):

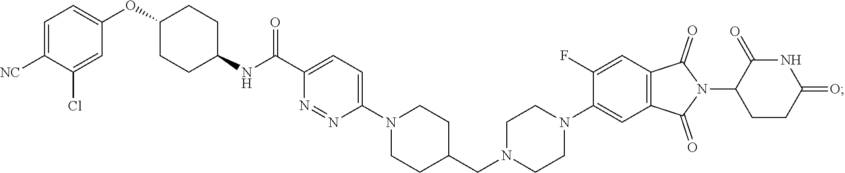 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating adenocarcinoma of the prostate in a subject in need thereof, comprising:
(i) oral, once daily administration of about 400 mg to about 500 mg of the compound of Formula (I-g):

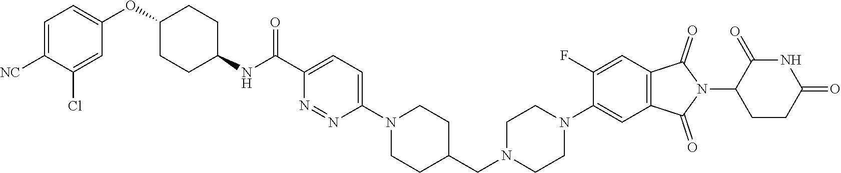 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating adenocarcinoma of the prostate in a subject in need thereof, comprising:
(i) oral, once daily administration of about 400 mg to about 500 mg of the compound of Formula (I-g):

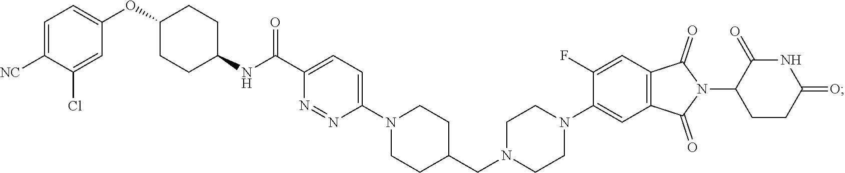 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating adenocarcinoma of the prostate in a subject in need thereof, comprising:
(i) oral, once daily administration of about 400 mg to about 500 mg of the compound of Formula (I-g):

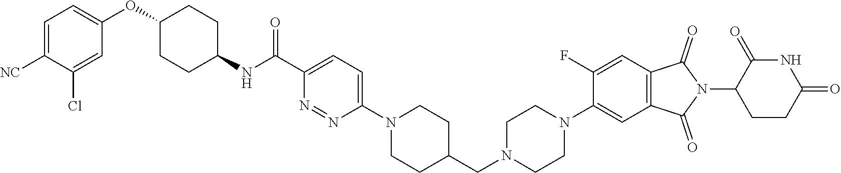 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating adenocarcinoma of the prostate in a subject in need thereof, comprising:
(i) oral, once daily administration of about 400 mg to about 500 mg of the compound of Formula (I-g):

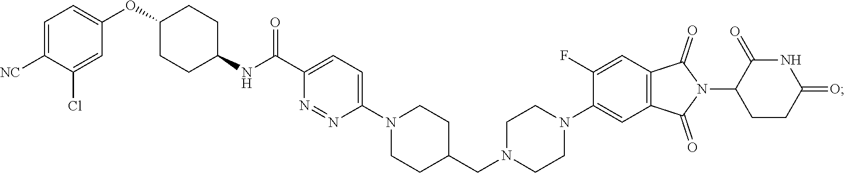 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic prostate cancer in a subject in need thereof, comprising:
(i) oral, once daily administration of about 400 mg to about 500 mg of the compound of Formula (I-g):

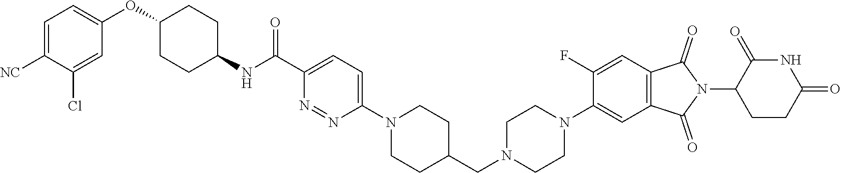 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic prostate cancer in a subject in need thereof, comprising:
(i) oral, once daily administration of about 400 mg to about 500 mg of the compound of Formula (I-g):

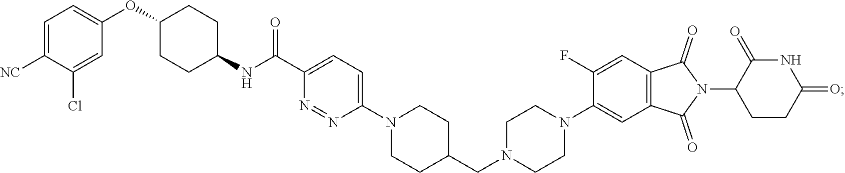 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic prostate cancer in a subject in need thereof, comprising:
(i) oral, once daily administration of about 400 mg to about 500 mg of the compound of Formula (I-g):

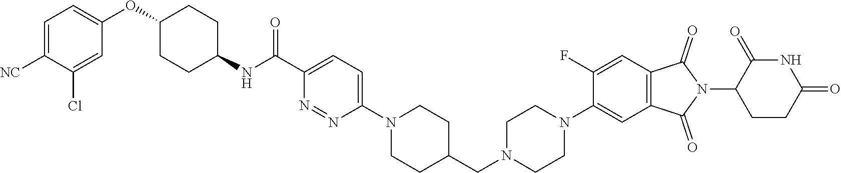 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer in a subject in need thereof, comprising:
(i) oral, once daily administration of about 400 mg to about 500 mg of the compound of Formula (I-g):

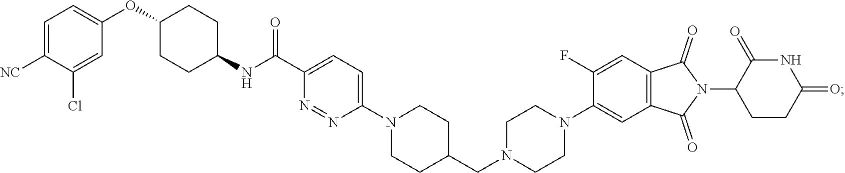 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-resistant prostate cancer in a subject in need thereof, comprising:
(i) oral, once daily administration of about 400 mg to about 500 mg of the compound of Formula (I-g):

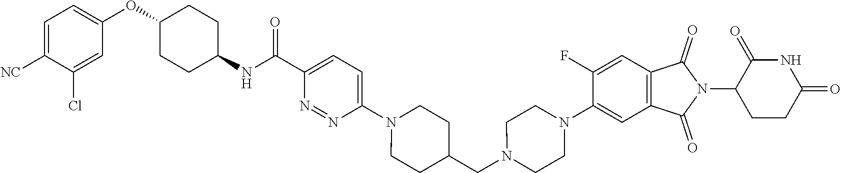 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating castrate-resistant prostate cancer in a subject in need thereof, comprising:
(i) oral, once daily administration of about 400 mg to about 500 mg of the compound of Formula (I-g):

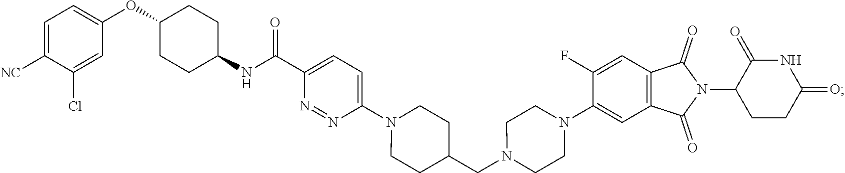 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating castrate-resistant prostate cancer in a subject in need thereof, comprising:
(i) oral, once daily administration of about 400 mg to about 500 mg of the compound of Formula (I-g):

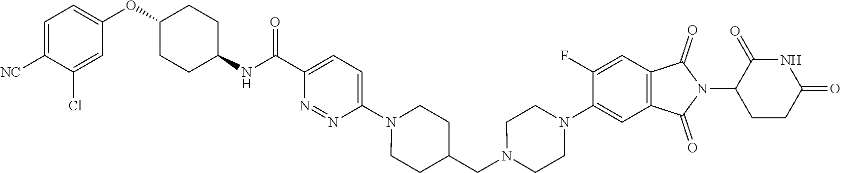 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating castrate-resistant prostate cancer in a subject in need thereof, comprising:
(i) oral, once daily administration of about 400 mg to about 500 mg of the compound of Formula (I-g):

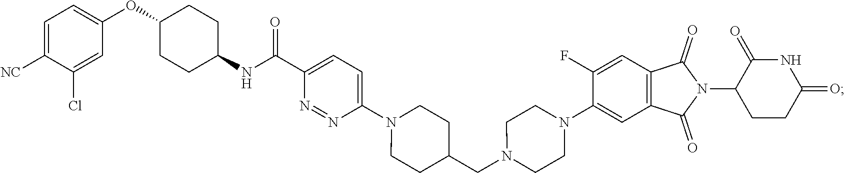 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-resistant prostate cancer in a subject in need thereof, comprising:
(i) oral, once daily administration of about 400 mg to about 500 mg of the compound of Formula (I-g):

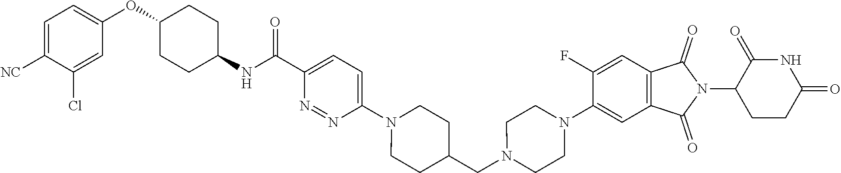 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-resistant prostate cancer in a subject in need thereof, comprising:
(i) oral, once daily administration of about 400 mg to about 500 mg of the compound of Formula (I-g):

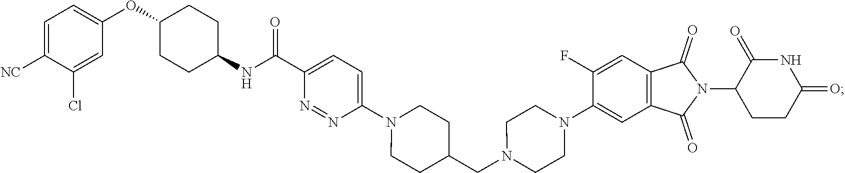 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-resistant prostate cancer in a subject in need thereof, comprising:
(i) oral, once daily administration of about 400 mg to about 500 mg of the compound of Formula (I-g):

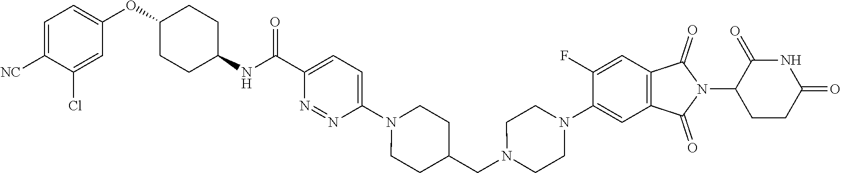 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-resistant prostate cancer in a subject in need thereof, comprising:
(i) oral, once daily administration of about 400 mg to about 500 mg of the compound of Formula (I-g):

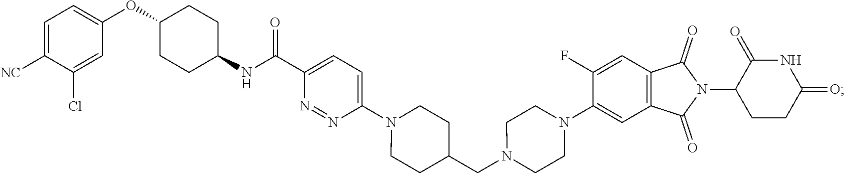 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating progressive mCRPC in a subject in need thereof, comprising:
(i) oral, once daily administration of about 400 mg to about 500 mg of the compound of Formula (I-g):

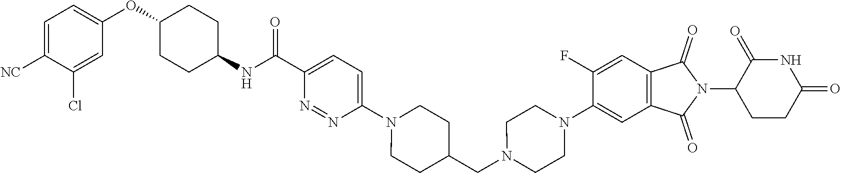 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

(ii) oral, once daily administration of about 250-1500 mg abiraterone acetate, or a pharmaceutically acceptable salt thereof.

In one aspect, the present application pertains to a method of treating progressive mCRPC in a subject in need thereof, comprising:
(i) oral, once daily administration of about 400 mg to about 500 mg of the compound of Formula (I-g):

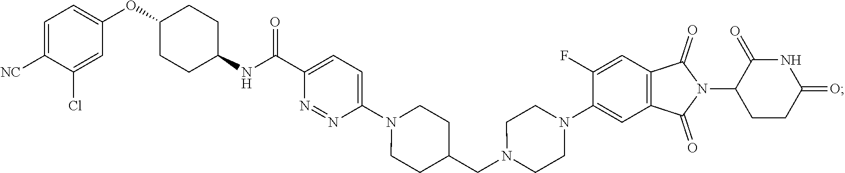 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and;
(ii) oral, once daily administration of about 250-1500 mg abiraterone acetate.

In one aspect, the present application pertains to a method of treating progressive mCRPC in a subject in need thereof, comprising:
(i) oral, once daily administration of about 400 mg to about 500 mg of the compound of Formula (I-g):

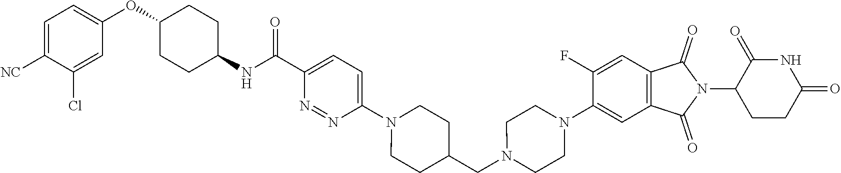 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating progressive mCRPC in a subject in need thereof, comprising:
(i) oral, once daily administration of about 400 mg to about 500 mg of the compound of Formula (I-g):

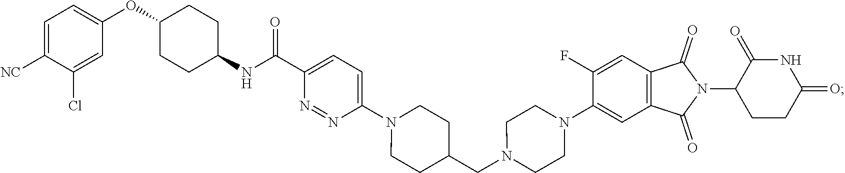 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-sensitive prostate cancer in a subject in need thereof, comprising:
(i) oral, once daily administration of about 400 mg to about 500 mg of the compound of Formula (I-g):

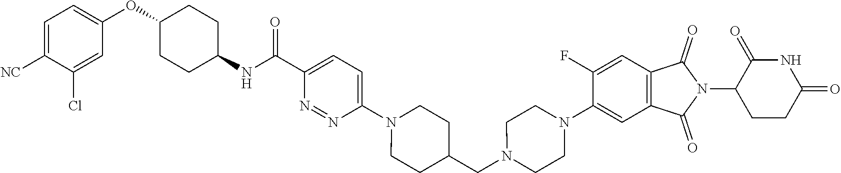 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating castrate-sensitive prostate cancer in a subject in need thereof, comprising:
(i) oral, once daily administration of about 400 mg to about 500 mg of the compound of Formula (I-g):

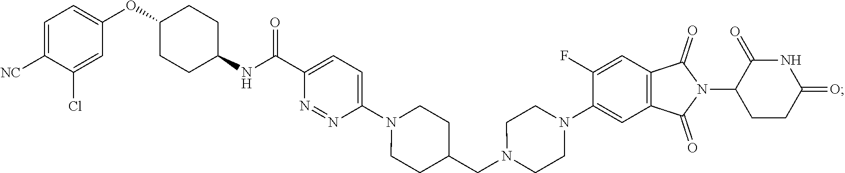 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating castrate-sensitive prostate cancer in a subject in need thereof, comprising:
(i) oral, once daily administration of about 400 mg to about 500 mg of the compound of Formula (I-g):

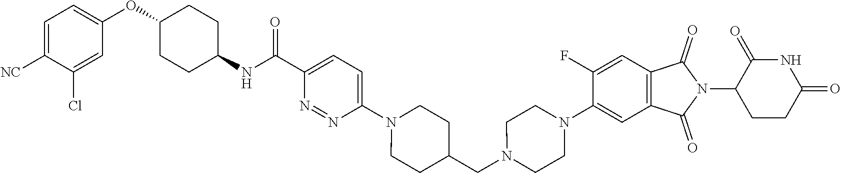 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating castrate-sensitive prostate cancer in a subject in need thereof, comprising:
(i) oral, once daily administration of about 400 mg to about 500 mg of the compound of Formula (I-g):

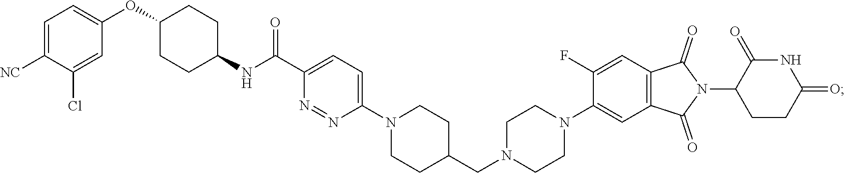 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-sensitive prostate cancer in a subject in need thereof, comprising:
(i) oral, once daily administration of about 400 mg to about 500 mg of the compound of Formula (I-g):

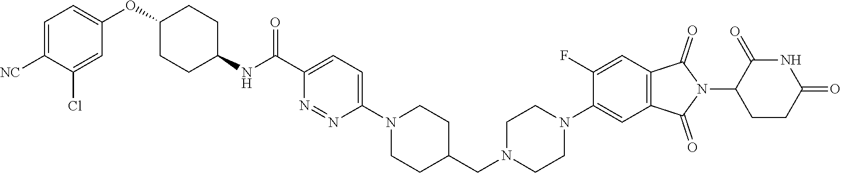 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-sensitive prostate cancer in a subject in need thereof, comprising:
(i) oral, once daily administration of about 400 mg to about 500 mg of the compound of Formula (I-g):

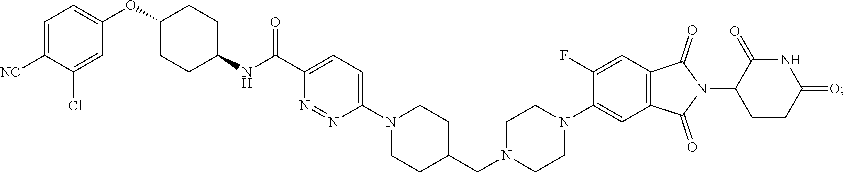 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-sensitive prostate cancer in a subject in need thereof, comprising:
(i) oral, once daily administration of about 400 mg to about 500 mg of the compound of Formula (I-g):

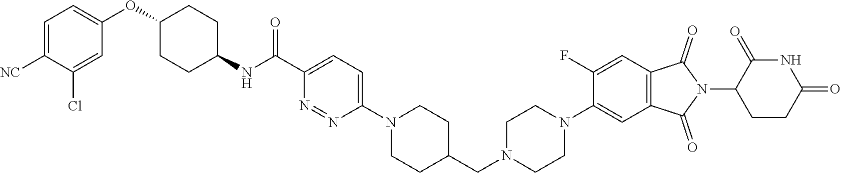 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-sensitive prostate cancer in a subject in need thereof, comprising:
(i) oral, once daily administration of about 400 mg to about 500 mg of the compound of Formula (I-g):

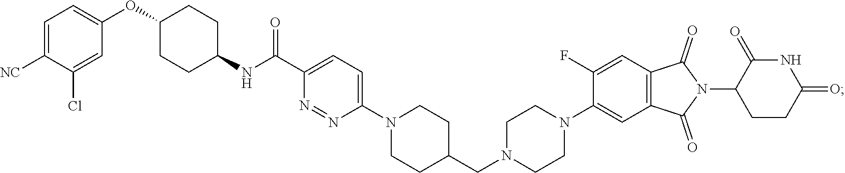 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating prostate cancer naïve to novel hormonal agents (NHA) in a subject in need thereof, comprising:
(i) oral, once daily administration of about 400 mg to about 500 mg of the compound of Formula (I-g):

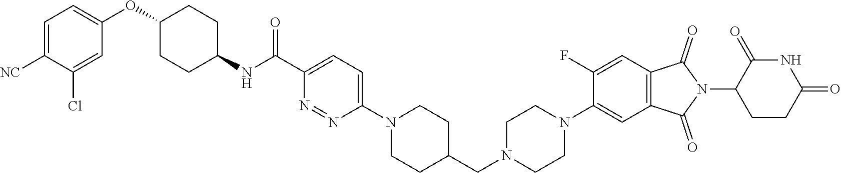 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating prostate cancer naïve to novel hormonal agents (NHA) in a subject in need thereof, comprising:
(i) oral, once daily administration of about 400 mg to about 500 mg of the compound of Formula (I-g):

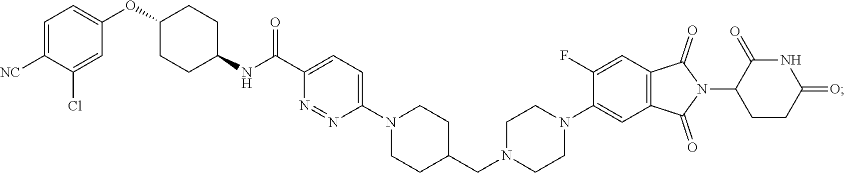 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating prostate cancer naïve to novel hormonal agents (NHA) in a subject in need thereof, comprising:
(i) oral, once daily administration of about 400 mg to about 500 mg of the compound of Formula (I-g):

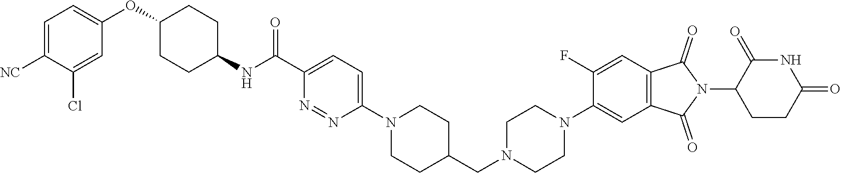 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating prostate cancer naïve to novel hormonal agents (NHA) in a subject in need thereof, comprising:
(i) oral, once daily administration of about 400 mg to about 500 mg of the compound of Formula (I-g):

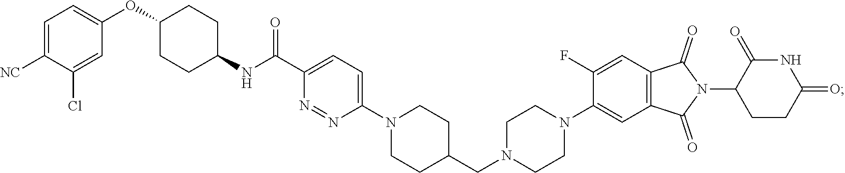 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic prostate cancer naïve to novel hormonal agents (NHA) in a subject in need thereof, comprising:
(i) oral, once daily administration of about 400 mg to about 500 mg of the compound of Formula (I-g):

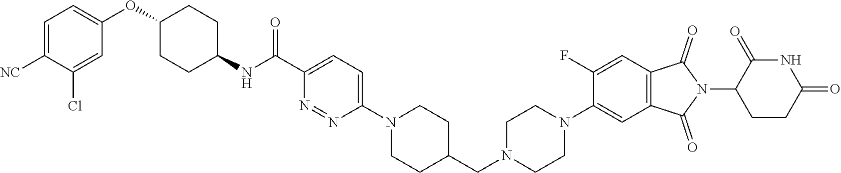 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic prostate cancer naïve to novel hormonal agents (NHA) in a subject in need thereof, comprising:
(i) oral, once daily administration of about 400 mg to about 500 mg of the compound of Formula (I-g):

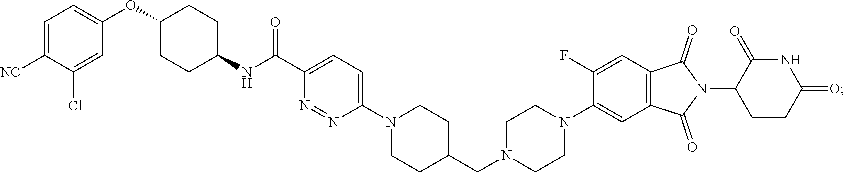 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic prostate cancer naïve to novel hormonal agents (NBA) in a subject in need thereof, comprising:
(i) oral, once daily administration of about 400 mg to about 500 mg of the compound of Formula (I-g):

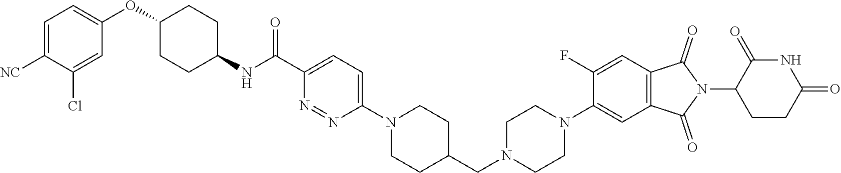 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic prostate cancer naïve to novel hormonal agents (NHA) in a subject in need thereof, comprising:
(i) oral, once daily administration of about 400 mg to about 500 mg of the compound of Formula (I-g):

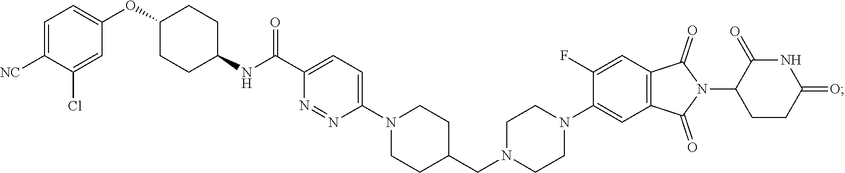 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-resistant prostate cancer naïve to novel hormonal agents (NHA) in a subject in need thereof, comprising:
(i) oral, once daily administration of about 400 mg to about 500 mg of the compound of Formula (I-g):

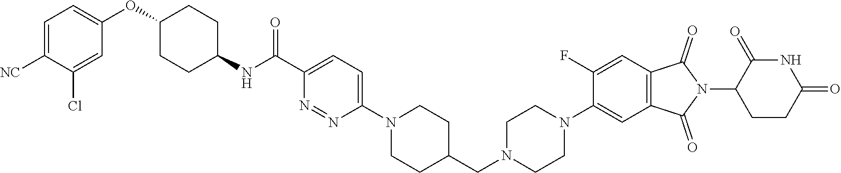 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating castrate-resistant prostate cancer naïve to novel hormonal agents (NHA) in a subject in need thereof, comprising:
(i) oral, once daily administration of about 400 mg to about 500 mg of the compound of Formula (I-g):

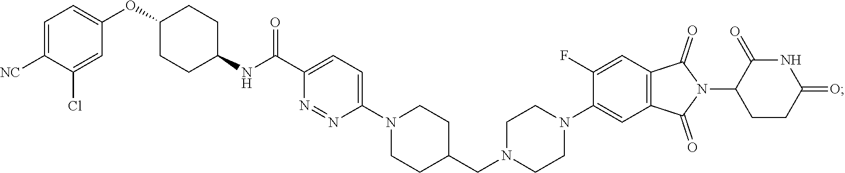 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating castrate-resistant prostate cancer naïve to novel hormonal agents (NHA) in a subject in need thereof, comprising:
(i) oral, once daily administration of about 400 mg to about 500 mg of the compound of Formula (I-g):

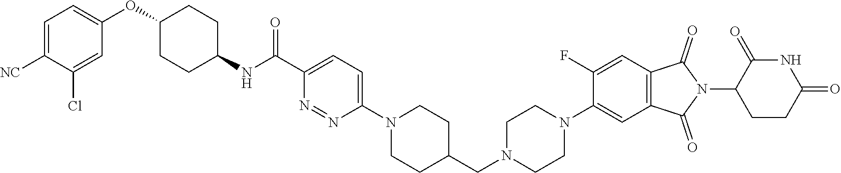 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating castrate-resistant prostate cancer naïve to novel hormonal agents (NHA) in a subject in need thereof, comprising:
(i) oral, once daily administration of about 400 mg to about 500 mg of the compound of Formula (I-g):

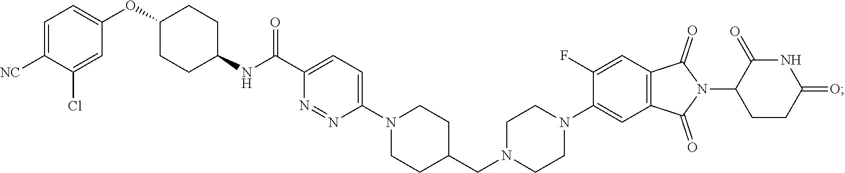 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating castrate-sensitive prostate cancer naïve to novel hormonal agents (NHA) in a subject in need thereof, comprising:
(i) oral, once daily administration of about 400 mg to about 500 mg of the compound of Formula (I-g):

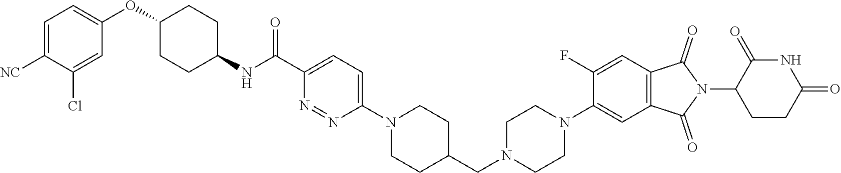 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

(ii) oral, once daily administration of about 250-1500 mg abiraterone acetate, or a pharmaceutically acceptable salt thereof.

In one aspect, the present application pertains to a method of treating castrate-sensitive prostate cancer naïve to novel hormonal agents (NHA) in a subject in need thereof, comprising:
(i) oral, once daily administration of about 400 mg to about 500 mg of the compound of Formula (I-g):

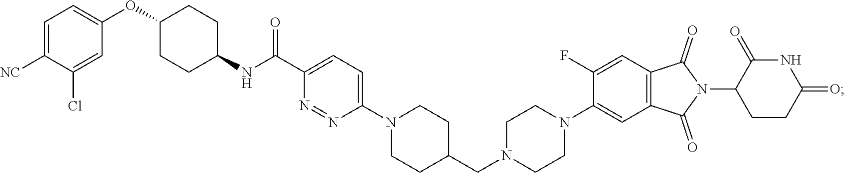 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and;
(ii) oral, once daily administration of about 250-1500 mg abiraterone acetate.

In one aspect, the present application pertains to a method of treating castrate-sensitive prostate cancer naïve to novel hormonal agents (NHA) in a subject in need thereof, comprising:
(i) oral, once daily administration of about 400 mg to about 500 mg of the compound of Formula (I-g):

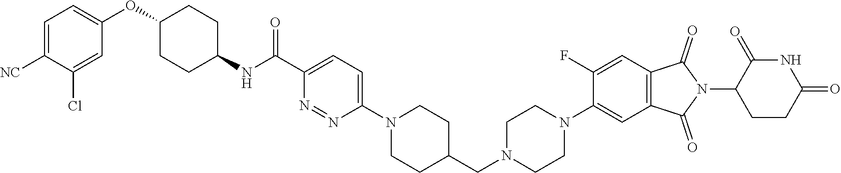 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating castrate-sensitive prostate cancer naïve to novel hormonal agents (NHA) in a subject in need thereof, comprising:
(i) oral, once daily administration of about 400 mg to about 500 mg of the compound of Formula (I-g):

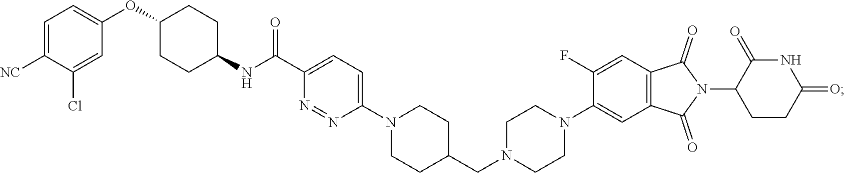 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-resistant prostate cancer naïve to novel hormonal agents (NHA) in a subject in need thereof, comprising:
(i) oral, once daily administration of about 400 mg to about 500 mg of the compound of Formula (I-g):

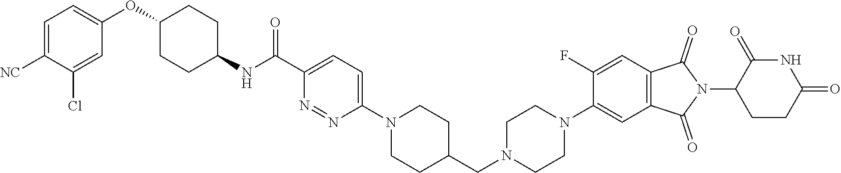 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-resistant prostate cancer naïve to novel hormonal agents (NHA) in a subject in need thereof, comprising:
(i) oral, once daily administration of about 400 mg to about 500 mg of the compound of Formula (I-g):

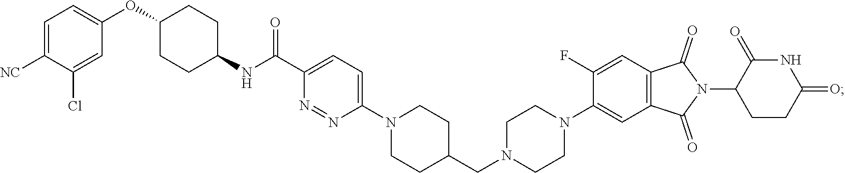 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-resistant prostate cancer naïve to novel hormonal agents (NHA) in a subject in need thereof, comprising:
(i) oral, once daily administration of about 400 mg to about 500 mg of the compound of Formula

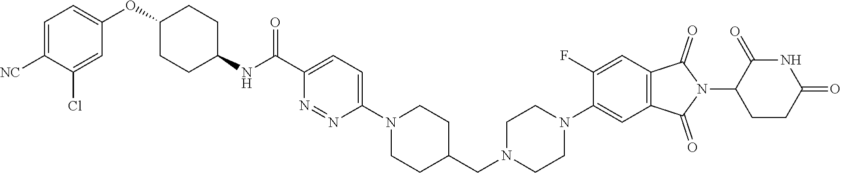 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-resistant prostate cancer naïve to novel hormonal agents (NHA) in a subject in need thereof, comprising:
(i) oral, once daily administration of about 400 mg to about 500 mg of the compound of Formula (I-g):

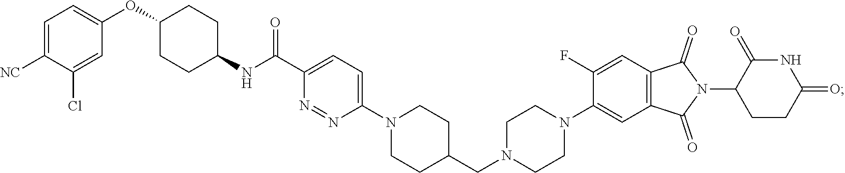 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the present application pertains to a method of treating metastatic castrate-sensitive prostate cancer naïve to novel hormonal agents (NHA) in a subject in need thereof, comprising:
(i) oral, once daily administration of about 400 mg to about 500 mg of the compound of Formula (I-g):

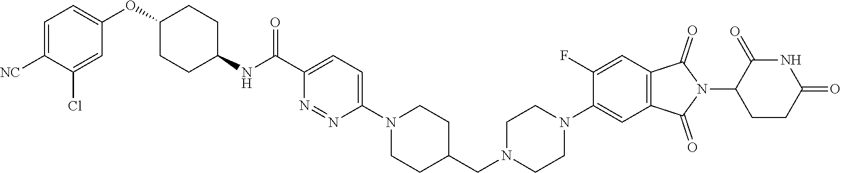 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 250-1500 mg abiraterone acetate, or a pharmaceutically acceptable salt thereof.

In one aspect, the present application pertains to a method of treating metastatic castrate-sensitive prostate cancer naïve to novel hormonal agents (NHA) in a subject in need thereof, comprising:
(i) oral, once daily administration of about 400 mg to about 500 mg of the compound of Formula (I-g):

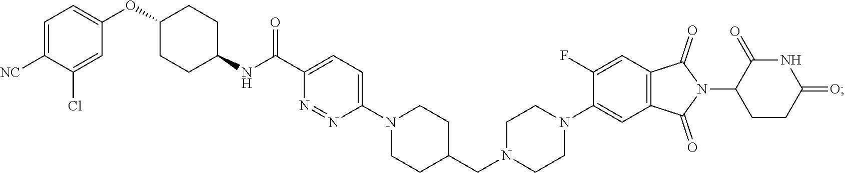 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 250-1500 mg abiraterone acetate.

In one aspect, the present application pertains to a method of treating metastatic castrate-sensitive prostate cancer naïve to novel hormonal agents (NHA) in a subject in need thereof, comprising:
(i) oral, once daily administration of about 400 mg to about 500 mg of the compound of Formula (I-g):

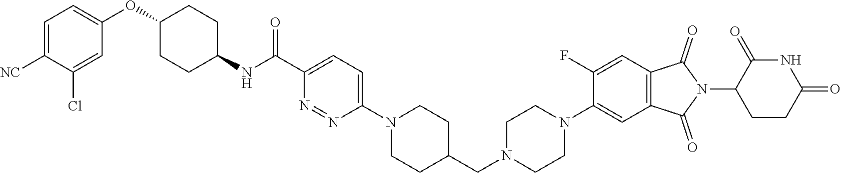 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.

In one aspect, the present application pertains to a method of treating metastatic castrate-sensitive prostate cancer naïve to novel hormonal agents (NHA) in a subject in need thereof, comprising:
(i) oral, once daily administration of about 400 mg to about 500 mg of the compound of Formula

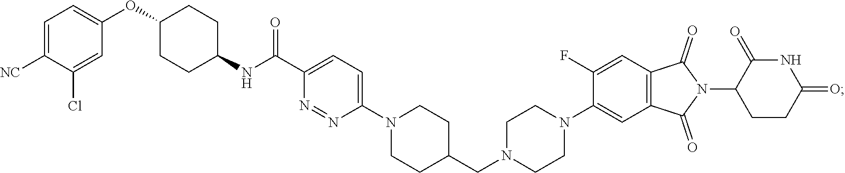 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate.

In one aspect, the methods of the disclosure further comprise administering a corticosteroid to the subject. In some embodiments, about 0.1 mg to about 20 mg of the corticosteroid is administered to the subject. In some embodiments, about 0.25 mg to about 10 mg of the corticosteroid is administered to the subject. In some embodiments, about 0.5 mg to about 5 mg of the corticosteroid is administered to the subject. In some embodiments, about 0.5 mg, about 1 mg, about 1.5 mg, about 2 mg, about 2.5 mg, about 3 mg, about 3.5 mg, about 4 mg, about 4.5 mg, or about 5 mg of the corticosteroid is administered to the subject. In some embodiments, the corticosteroid is orally administered to the subject once or twice daily. In some embodiments, the corticosteroid is orally administered to the subject once daily. In some embodiments, the corticosteroid is orally administered to the subject twice daily. In some embodiments, the corticosteroid is a glucocorticoid. In some embodiments, the corticosteroid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclometasone. In some embodiments, the corticosteroid is prednisone. In some embodiments, the corticosteroid is prednisolone.

In one aspect, the methods of the disclosure further comprise administering a glucocorticoid or mineralocorticoid to the subject. In some embodiments, about 0.1 mg to about 20 mg of the glucocorticoid or mineralocorticoid is administered to the subject. In some embodiments, about 0.25 mg to about 10 mg of the glucocorticoid or mineralocorticoid is administered to the subject. In some embodiments, about 0.5 mg to about 5 mg of the glucocorticoid or mineralocorticoid is administered to the subject. In some embodiments, about 0.5 mg, about 1 mg, about 1.5 mg, about 2 mg, about 2.5 mg, about 3 mg, about 3.5 mg, about 4 mg, about 4.5 mg, or about 5 mg of the glucocorticoid or mineralocorticoid is administered to the subject. In some embodiments, the glucocorticoid or mineralocorticoid is orally administered to the subject once or twice daily. In some embodiments, the glucocorticoid or mineralocorticoid is orally administered to the subject once daily. In some embodiments, the glucocorticoid or mineralocorticoid is orally administered to the subject twice daily. In some embodiments, the glucocorticoid or mineralocorticoid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclometasone.

In one aspect, the methods of the disclosure further comprise administering prednisone to the subject. In some embodiments, about 0.1 mg to about 20 mg of prednisone is administered to the subject. In some embodiments, about 0.25 mg to about 10 mg of prednisone is administered to the subject. In some embodiments, about 0.5 mg to about 5 mg of prednisone is administered to the subject. In some embodiments, about 0.5 mg, about 1 mg, about 1.5 mg, about 2 mg, about 2.5 mg, about 3 mg, about 3.5 mg, about 4 mg, about 4.5 mg, or about 5 mg of prednisone is administered to the subject. In some embodiments, the prednisone is orally administered to the subject once or twice daily. In some embodiments, the prednisone is orally administered to the subject once daily. In some embodiments, the prednisone is orally administered to the subject twice daily.

In one aspect, the methods of the disclosure further comprise administering prednisolone to the subject. In some embodiments, about 0.1 mg to about 20 mg of prednisolone is administered to the subject. In some embodiments, about 0.25 mg to about 10 mg of prednisolone is administered to the subject. In some embodiments, about 0.5 mg to about 5 mg of prednisolone is administered to the subject. In some embodiments, about 0.5 mg, about 1 mg, about 1.5 mg, about 2 mg, about 2.5 mg, about 3 mg, about 3.5 mg, about 4 mg, about 4.5 mg, or about 5 mg of prednisolone is administered to the subject. In some embodiments, the prednisolone is orally administered to the subject once or twice daily. In some embodiments, the prednisolone is orally administered to the subject once daily. In some embodiments, the prednisolone is orally administered to the subject twice daily.

In one aspect, the methods of the disclosure further comprise orally administering about 2.5 mg or about 5 mg of prednisone or prednisolone to the subject, once or twice daily.

In one aspect, the methods of the disclosure further comprise orally administering about 5 mg of prednisone to the subject, once daily.

In one aspect, the methods of the disclosure further comprise orally administering about 5 mg of prednisolone to the subject, once daily.

In some embodiments, the prostate cancer is not prostate cancer naïve to novel hormonal agents (NHA). In some embodiments, the prostate cancer that is not prostate cancer naïve to novel hormonal agents (NHA) is also metastatic prostate cancer, castrate-resistant prostate cancer, metastatic castrate-resistant prostate cancer, castrate-sensitive prostate cancer, or metastatic castrate-sensitive prostate cancer.

In some embodiments, the subject has a histological, pathological, or cytological confirmed diagnosis of adenocarcinoma of the prostate.

In some embodiments, the subject has ongoing treatment with stable doses of abiraterone (on an empty stomach) and a concomitant corticosteroid for mCRPC or metastatic castration-sensitive prostate cancer (mCSPC) until Cycle 1, Day 1 (C1D1). If a patient had treatment interruptions or dose modifications of abiraterone within 2 weeks of C1D1, the Investigator would inform the Medical Monitor (or designee).

In some embodiments, the patient has recent PSA values with:
(a) Rising PSAs no less than 16 weeks after initiation of abiraterone; and
(b) At least 2 rising PSA values that are higher than the PSA nadir on abiraterone, measured at a minimum of 1 week apart. The screening PSA for this study may be used as the 2^{nd} PSA value.

In some embodiments, the subject suffering from prostate cancer (e.g., mCRPC) is also undergoing ongoing androgen deprivation therapy (ADT). In some embodiments, ADT comprises the administration of a gonadotropin releasing hormone analog or inhibitor to the subject. In some embodiments, the gonadotropin releasing hormone analog or inhibitor is leuprolide, goserelin, triptorelin, histrelin, leuprolide mesylate, or a pharmaceutically acceptable salt thereof. In some embodiments, the gonadotropin releasing hormone analog or inhibitor is leuprolide. In some embodiments, the gonadotropin releasing hormone analog or inhibitor is leuprolide mesylate. In some embodiments, the gonadotropin releasing hormone analog or inhibitor is goserelin. In some embodiments, the gonadotropin releasing hormone analog or inhibitor is goserelin acetate. In some embodiments, the gonadotropin releasing hormone analog or inhibitor is triptorelin. In some embodiments, the gonadotropin releasing hormone analog or inhibitor is triptorelin acetate. In some embodiments, the gonadotropin releasing hormone analog or inhibitor is triptorelin pamoate. In some embodiments, the gonadotropin releasing hormone analog or inhibitor is histrelin. In some embodiments, the gonadotropin releasing hormone analog or inhibitor is histrelin acetate. In some embodiments, the subject has undergone an orchiectomy.

In some embodiments, prior to the administration of the therapeutically effective amount of a compound of Formula (I) (preferably, the compound of Formula (I-g)), the subject experienced progression of prostate cancer on at least one prior approved systemic therapy for metastatic prostate cancer. In some embodiments, prior to the administration of the therapeutically effective amount of a bifunctional compound of the present disclosure, the subject experienced progression of prostate cancer on at least two prior approved systemic therapies for metastatic prostate cancer. In some embodiments, at least one of the prior approved systemic therapies for metastatic prostate cancer is a second-generation androgen inhibitor. In some embodiments, prior to the administration of the therapeutically effective amount of a bifunctional compound of the present disclosure, the subject experienced progression of prostate cancer on at least two prior approved systemic therapies for metastatic prostate cancer, at least one of which is a second generation androgen inhibitor. In some embodiments, the second-generation androgen inhibitor is abiraterone, enzalutamide, darolutamide, apalutamide, or a pharmaceutically acceptable salt thereof. In some embodiments, the second-generation androgen inhibitor is abiraterone. In some embodiments, the second-generation androgen inhibitor is abiraterone acetate. In some embodiments, the second-generation androgen inhibitor is enzalutamide. In some embodiments, the second-generation androgen inhibitor is darolutamide. In some embodiments, the second-generation androgen inhibitor is apalutamide.

In some embodiments, prior to the administration of the therapeutically effective amount of a compound of Formula (I) (preferably, the compound of Formula (I-g)), the subject received at least one but not more than three prior second generation anti-androgen agents (e.g., enzalutamide, abiraterone, darolutamide, or apalutamide). In some embodiments, the subject received one prior second generation anti-androgen agent. In some embodiments, the subject received two prior second generation anti-androgen agents. In some embodiments, the subject received three prior second generation anti-androgen agents.

In some embodiments, prior to the administration of the therapeutically effective amount of a compound of Formula (I) (preferably, the compound of Formula (I-g)), the subject received no more than two prior chemotherapy regimens. In some embodiments, the subject received two prior chemotherapy regimens. In some embodiments, the subject received one prior chemotherapy regimen. In some embodiments, the subject has not received a prior chemotherapy regimen.

In some embodiments, the subject has an ECOG performance status of 0 or 1. In some embodiments, the subject has an ECOG performance status of 1. In some embodiments, the subject has an ECOG performance status of 0.

In some embodiments, the subject does not have a symptomatic brain metastasis requiring steroids above physiologic replacement doses.

In some embodiments, the subject does not have active inflammatory bowel disease.

In some embodiments, the subject does not have chronic diarrhea.

In some embodiments, the subject does not have diverticular disease.

In some embodiments, the subject has not previously undergone gastric resection.

In some embodiments, the subject has not previously undergone lap band surgery.

In some embodiments, the subject has not previously undergone radiation therapy within four weeks prior to the initial the administration of a compound of Formula (I) (preferably, the compound of Formula (I-g)).

In some embodiments, the subject has not previously undergone radiation therapy where greater than about 25% of the patient's bone marrow was irradiated.

In some embodiments, the subject has not been administered an investigational drug within four weeks prior to the initial the administration of a compound of Formula (I) (preferably, the compound of Formula (I-g)).

In some embodiments, the subject has not received a systemic anti-cancer therapy within 2 weeks of the first administration of the therapeutically effective amount of a compound of Formula (I) (preferably, the compound of Formula (I-g)). In some embodiments, the subject has not received a systemic anti-cancer therapy within 2 weeks of the first administration of the therapeutically effective amount of a bifunctional compound of the present disclosure, except for agents to maintain castration status.

In some embodiments, the subject has not received bicalutamide within 6 weeks of the first administration of the therapeutically effective amount of a compound of Formula (I) (preferably, the compound of Formula (I-g)).

In some embodiments, the subject has not received mitomycin C within 6 weeks of the first administration of the therapeutically effective amount of a compound of Formula (I) (preferably, the compound of Formula (I-g)).

In some embodiments, the subject has not received nitrosoureas within 6 weeks of the first administration of the therapeutically effective amount of a compound of Formula (I) (preferably, the compound of Formula (I-g)).

In some embodiments, the subject has not received abiraterone within 4 weeks of the first administration of the therapeutically effective amount of a compound of Formula (I) (preferably, the compound of Formula (I-g)).

In some embodiments, the subject has not been taking agents that are either a) sensitive P-glycoprotein (P-gp), breast cancer resistance protein (BCRP), or CYP3A4 substrates, b) P-gp, BCRP, CYP3A4, or CYP2D6 substrates that have a narrow therapeutic index, and/or c) strong CYP3A4 inhibitors or inducers.

In some embodiments, the subject has not undergone major surgery within 4 weeks of first dose of study drug.

In some embodiments, the subject has not had any of the following in the previous 12 months: myocardial infarction, severe/unstable angina, coronary/peripheral artery bypass graft, symptomatic congestive heart failure (New York Heart Association class II, III or IV), cerebrovascular accident, transient ischemic attack, symptomatic pulmonary embolism, or other clinically significant episode of thromboembolic disease.

In some embodiments, the subject has not had any of the following in the previous 6 months: congenital long QT syndrome, Torsade de Pointes, arrhythmias (including sustained ventricular tachyarrhythmia and ventricular fibrillation), left anterior hemiblock (bifascicular block), or ongoing cardiac dysrhythmias of National Cancer Institute (NCI) Common Toxicity Criteria for Adverse Events (CTCAE) Grade ≥2, atrial fibrillation of any grade (Grade ≥2 in the case of asymptomatic lone atrial fibrillation).

In some embodiments, the subject does not have hypertension that cannot be controlled by medications (>150/90 mmHg despite optimal medical therapy).

In some embodiments, the subject does not have active, uncontrolled bacterial, fungal, or viral infection, including hepatitis B virus, hepatitis C virus, known human immunodeficiency virus (HV), or acquired immunodeficiency syndrome (AIDS)-related illness. Patients whose viral load is negative or HIV-seropositive patients who are healthy and low risk for AIDS-related outcomes may be considered eligible. HIV-positive patients should be free of any serious AIDS-defining conditions (e.g., opportunistic infections) with an undetectable viral load.

In some embodiments, the subject does not have active inflammatory gastrointestinal disease, uncontrolled chronic diarrhea, known diverticular disease, or previous gastric resection or lap band surgery. Gastroesophageal reflux disease is allowed provided that treatment with prohibited medications (e.g., proton pump inhibitors) is not required.

In some embodiments, the subject does not have Child Pugh C.

In some embodiments, the subject does not have electrolyte imbalances of hypokalemia, hypomagnesemia, and/or hypocalcemia.

In some embodiments, the subject does not have corrected QT interval by Fredericia's method (QTcF)≥470 msec.

In some embodiments, the subject does not have untreated brain metastases or brain metastases requiring steroids above physiologic replacement doses. Patients with previously diagnosed brain metastases are eligible if they have completed definitive treatment and have recovered from the acute effects of radiation therapy or surgery prior to first dose of study drug, have discontinued high-dose corticosteroid treatment for these metastases for at least 4 weeks prior to C1D1, and are neurologically stable as judged by the Investigator.

In some embodiments, the subject does not have any other active malignancy within 3 years prior to enrollment, except for adequately treated basal cell or squamous cell skin cancer, or carcinoma in situ. Other localized malignancies considered highly curable by the Investigator are eligible.

In some embodiments, the subject does not have suspected hypersensitivity to Compound (I-g).

In some embodiments, the subject does not have a history of allergy or reaction to any of the drug components.

In some embodiments, the subject suffering from any of the types of prostate cancer disclosed herein will have a different response to treatment with a compound of Formula (I) (preferably, the compound of Formula (I-g)), or a pharmaceutically acceptable salt depending on the AR biomarker status of the subject.

In one aspect, the application pertains to treating prostate cancer with a compound of Formula (I), wherein the compound of Formula (I) refers to a compound with the following structure:

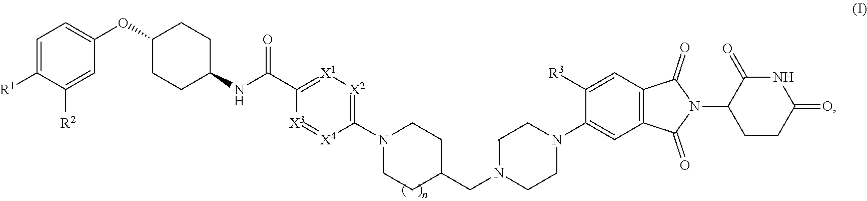

or a pharmaceutically acceptable salt thereof, wherein R¹, R², R³, X¹, X², X³, and X⁴ and n are defined herein. In some embodiments, the cancer is metastatic prostate cancer. In some embodiments, the cancer is castrate-resistant or castration-resistant prostate cancer. In some embodiments, the cancer is metastatic, castrate-resistant prostate cancer. In some embodiments, the prostate cancer is castrate-sensitive prostate cancer. In some embodiments, the prostate cancer is metastatic castrate-sensitive prostate cancer. In some embodiments, the prostate cancer is prostate cancer naïve to novel hormonal agents (NHA). In some embodiments, the prostate cancer is metastatic prostate cancer naïve to novel hormonal agents (NHA). In some embodiments, the prostate cancer is castrate-resistant prostate cancer naïve to novel hormonal agents (NHA). In some embodiments, the prostate cancer is castrate-sensitive prostate cancer naïve to novel hormonal agents (NHA). In some embodiments, the prostate cancer is metastatic castrate-resistant prostate cancer naïve to novel hormonal agents (NHA). In some embodiments, the prostate cancer is metastatic castrate-sensitive prostate cancer naïve to novel hormonal agents (NBA).

In one aspect, the application pertains to treating prostate cancer with a compound of Formula (I), wherein the compound of Formula (I) refers to a compound with the following structure:

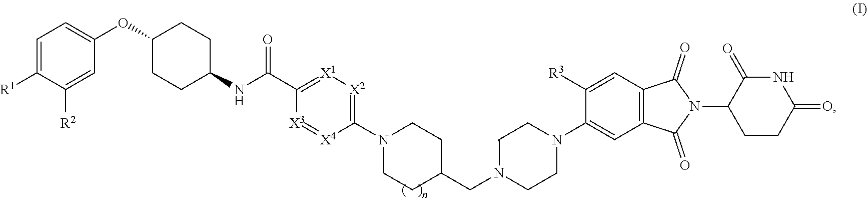

wherein R¹, R², R³, X¹, X², X³, and X⁴ and n are defined herein. In some embodiments, the cancer is metastatic prostate cancer. In some embodiments, the cancer is castrate-resistant or castration-resistant prostate cancer. In some embodiments, the cancer is metastatic, castrate-resistant prostate cancer. In some embodiments, the prostate cancer is castrate-sensitive prostate cancer. In some embodiments, the prostate cancer is metastatic castrate-sensitive prostate cancer. In some embodiments, the prostate cancer is prostate cancer naïve to novel hormonal agents (NHA). In some embodiments, the prostate cancer is metastatic prostate cancer naïve to novel hormonal agents (NHA). In some embodiments, the prostate cancer is castrate-resistant prostate cancer naïve to novel hormonal agents (NHA). In some embodiments, the prostate cancer is castrate-sensitive prostate cancer naïve to novel hormonal agents (NHA). In some embodiments, the prostate cancer is metastatic castrate-resistant prostate cancer naïve to novel hormonal agents (NHA). In some embodiments, the prostate cancer is metastatic castrate-sensitive prostate cancer naïve to novel hormonal agents (NHA). In some embodiments, the compound of Formula (I) is the compound of Formula (I-g).

In one aspect, the application pertains to treating prostate cancer with a compound of Formula (I) in combination with abiraterone, a pharmaceutically acceptable salt of abiraterone, abiraterone acetate, or a pharmaceutically acceptable salt of abiraterone acetate, wherein the compound of Formula (I) refers to a compound with the following structure:

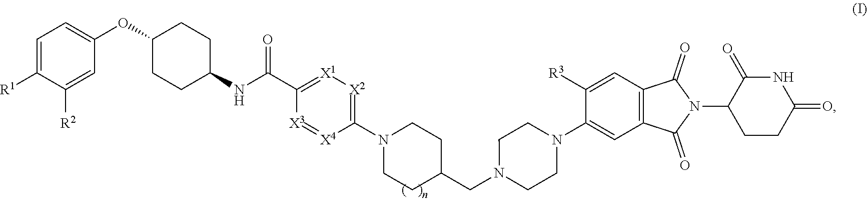

or a pharmaceutically acceptable salt, wherein R¹, R², R³, X¹, X², X³, and X⁴ and n are defined herein. In some embodiments, the cancer is metastatic prostate cancer. In some embodiments, the cancer is castrate-resistant or castration-resistant prostate cancer. In some embodiments, the cancer is metastatic, castrate-resistant prostate cancer. In some embodiments, the prostate cancer is castrate-sensitive prostate cancer. In some embodiments, the prostate cancer is metastatic castrate-sensitive prostate cancer. In some embodiments, the prostate cancer is prostate cancer naïve to novel hormonal agents (NHA). In some embodiments, the prostate cancer is metastatic prostate cancer naïve to novel hormonal agents (NHA). In some embodiments, the prostate cancer is castrate-resistant prostate cancer naïve to novel hormonal agents (NHA). In some embodiments, the prostate cancer is castrate-sensitive prostate cancer naïve to novel hormonal agents (NHA). In some embodiments, the prostate cancer is metastatic castrate-resistant prostate cancer naïve to novel hormonal agents (NHA). In some embodiments, the prostate cancer is metastatic castrate-sensitive prostate cancer naïve to novel hormonal agents (NHA). In some embodiments, the compound of Formula (I) is the compound of Formula (I-g).

In some embodiments, the compound of Formula (I) is the compound of Formula (I-g).

In one aspect, the application pertains to treating prostate cancer with a compound of Formula (I) in combination with abiraterone acetate, wherein the compound of Formula (I) refers to a compound with the following structure:

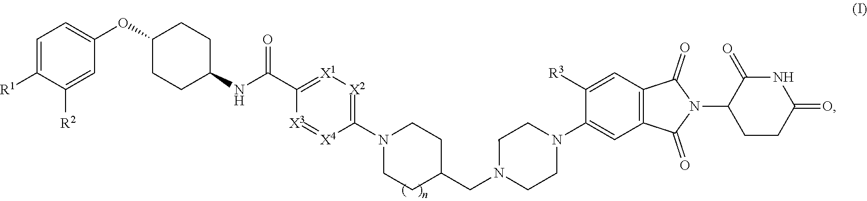

wherein R¹, R², R³, X¹, X², X³, and X⁴ and n are defined herein. In some embodiments, the cancer is metastatic prostate cancer. In some embodiments, the cancer is castrate-resistant or castration-resistant prostate cancer. In some embodiments, the cancer is metastatic, castrate-resistant prostate cancer. In some embodiments, the prostate cancer is castrate-sensitive prostate cancer. In some embodiments, the prostate cancer is metastatic castrate-sensitive prostate cancer. In some embodiments, the prostate cancer is prostate cancer naïve to novel hormonal agents (NBA). In some embodiments, the prostate cancer is metastatic prostate cancer naïve to novel hormonal agents (NBA). In some embodiments, the prostate cancer is castrate-resistant prostate cancer naïve to novel hormonal agents (NBA). In some embodiments, the prostate cancer is castrate-sensitive prostate cancer naïve to novel hormonal agents (NBA). In some embodiments, the prostate cancer is metastatic castrate-resistant prostate cancer naïve to novel hormonal agents (NBA). In some embodiments, the prostate cancer is metastatic castrate-sensitive prostate cancer naïve to novel hormonal agents (NBA). In some embodiments, the compound of Formula (I) is the compound of Formula (I-g).

In one aspect, treating cancer results in a reduction in size of a tumor. A reduction in size of a tumor may also be referred to as “tumor regression.” Preferably, after treatment, tumor size is reduced by 50 or greater relative to its size prior to treatment; more preferably, tumor size is reduced by 100% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active agent or compound. In another preferred aspect, an increase in average survival time of a population may also be measured, for example, by calculating for a population the average length of survival following completion of a first round of treatment with the compound of Formula (I-g) or with the combination of the compound of Formula (I-g) and abiraterone acetate.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active agent or compound. In another preferred aspect, an increase in average survival time of a population may also be measured, for example, by calculating for a population the average length of survival following completion of a first round of treatment with the compound of Formula (I-g) or with the combination of the compound of Formula (I-g) and abiraterone acetate.

In another aspect, treating cancer results in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured, for example, by measuring an increase in the diameter of a tumor after a prior tumor shrinkage that followed treatment. In another preferred aspect, a decrease in tumor regrowth is indicated by failure of tumors to reoccur after treatment has stopped.

The dosages of a compound of Formula (I) (preferably, the compound of Formula (I-g)) for any of the methods and uses described herein vary depending on the agent, the age, weight, and clinical condition of the recipient subject, and the experience and judgment of the clinician or practitioner administering the therapy, among other factors affecting the selected dosage.

The therapeutically effective amount of a compound of Formula (I) (preferably, the compound of Formula (I-g)) may be administered one or more times over a day for up to 30 or more days, followed by 1 or more days of non-administration of a compound of Formula (I). This type of treatment schedule, i.e., administration of a compound of Formula (I) on consecutive days followed by non-administration of a compound of Formula (I) on consecutive days may be referred to as a treatment cycle. A treatment cycle may be repeated as many times as necessary to achieve the intended affect.

In some embodiments, the therapeutically effective amount of a compound of Formula (I) (preferably, the compound of Formula (I-g)) is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer.

In some embodiments, the therapeutically effective amount of a compound of Formula (I) (preferably, the compound of Formula (I-g)) is about 10 to about 40 mg, about 20 to about 50 mg, about 30 to about 60 mg, about 40 to about 70 mg, about 50 to about 80 mg, about 60 to about 90 mg, about 70 to about 100 mg, about 80 to about 110 mg, about 90 to about 120 mg, about 100 to about 130 mg, about 110 to about 140 mg, about 120 to about 150 mg, about 130 to about 160 mg, about 140 to about 170 mg, about 150 to about 180 mg, about 160 to about 190 mg, about 170 to about 200 mg, about 180 to about 210 mg, about 190 to about 220 mg, about 200 to about 230 mg, about 210 to about 240 mg, about 220 to about 250 mg, about 230 to about 260 mg, about 240 to about 270 mg, about 250 to about 280 mg, about 260 to about 290 mg, about 270 to about 300 mg, about 280 to about 310 mg, about 290 to about 320 mg, about 300 to about 330 mg, about 310 to about 340 mg, about 320 to about 350 mg, about 330 to about 360 mg, about 340 to about 370 mg, about 350 to about 380 mg, about 360 to about 390 mg, about 370 to about 400 mg, about 380 to about 410 mg, about 390 to about 420 mg, about 400 to about 430 mg, about 410 to about 440 mg, about 420 to about 450 mg, about 430 to about 460 mg, about 440 to about 470 mg, about 450 to about 480 mg, about 460 to about 490 mg, about 470 to about 500 mg, about 480 to about 510 mg, about 490 to about 520 mg, about 500 to about 530 mg, about 510 to about 540 mg, about 520 to about 550 mg, about 530 to about 560 mg, about 540 to about 570 mg, about 550 to about 580 mg, about 560 to about 590 mg, about 570 to about 600 mg, about 580 to about 610 mg, about 590 to about 620 mg, about 600 to about 630 mg, about 610 to about 640 mg, about 620 to about 650 mg, about 630 to about 660 mg, about 640 to about 670 mg, about 650 to about 680 mg, about 660 to about 690 mg, about 670 to about 700 mg, about 680 to about 710 mg, about 690 to about 720 mg, about 700 to about 730 mg, about 710 to about 740 mg, about 720 to about 750 mg, about 730 to about 760 mg, about 740 to about 770 mg, about 750 to about 780 mg, about 760 to about 790 mg, about 770 to about 800 mg, about 780 to about 810 mg, about 790 to about 820 mg, about 800 to about 830 mg, about 810 to about 840 mg, about 820 to about 850 mg, about 830 to about 860 mg, about 840 to about 870 mg, about 850 to about 880 mg, about 860 to about 890 mg, about 870 to about 900 mg, about 880 to about 910 mg, about 890 to about 920 mg, about 900 to about 930 mg, about 910 to about 940 mg, about 920 to about 950 mg, about 930 to about 960 mg, about 940 to about 970 mg, about 950 to about 980 mg, about 960 to about 990 mg, or about 970 to about 1,000 mg administered once, twice, three times, four times, or more daily in single or divided doses (which dose may be adjusted for the subject's weight in kg, body surface area in m², and age in years).

In some embodiments, the therapeutically effective amount of a compound of Formula (I) (preferably, the compound of Formula (I-g)) is about 35 mg to about 1000 mg administered once, twice, three times, four times, or more daily in single or divided doses (which dose may be adjusted for the subject's weight in kg, body surface area in m², and age in years).

In some embodiments, the therapeutically effective amount of a compound of Formula (I) (preferably, the compound of Formula (I-g)) is about 70 mg to about 1000 mg administered once, twice, three times, four times, or more daily in single or divided doses (which dose may be adjusted for the subject's weight in kg, body surface area in m², and age in years).

In some embodiments, the therapeutically effective amount of a compound of Formula (I) (preferably, the compound of Formula (I-g)) is about 35 mg, 70 mg, 105 mg, 140 mg, 175 mg, 200 mg, 210 mg, 245 mg, 280 mg, 300 mg, 315 mg, 350 mg, 385 mg, 400 mg, 420 mg, 455 mg, 490 mg, 525 mg, 560 mg, 595 mg, 630 mg, 665 mg, or 700 mg administered once, twice, three times, four times, or more daily in single or divided doses (which dose may be adjusted for the subject's weight in kg, body surface area in m², and age in years).

The therapeutically effective amount of a compound of Formula (I) (preferably, the compound of Formula (I-g)) can also range from about 0.01 mg/kg per day to about 100 mg/kg per day. In an aspect, therapeutically effective amount of a compound of Formula (I) (preferably, the compound of Formula (I-g)) can range from about 0.05 mg/kg per day to about 10 mg/kg per day. In an aspect, therapeutically effective amount of a compound of Formula (I) (preferably, the compound of Formula (I-g)) can range from about 0.075 mg/kg per day to about 5 mg/kg per day. In an aspect, therapeutically effective amount of a compound of Formula (I) (preferably, the compound of Formula (I-g)) can range from about 0.10 mg/kg per day to about 1 mg/kg per day. In an aspect, therapeutically effective amount of a compound of Formula (I) (preferably, the compound of Formula (I-g)) can range from about 0.20 mg/kg per day to about 0.70 mg/kg per day.

In some embodiments, the therapeutically effective amount of a compound of Formula (I) (preferably, the compound of Formula (I-g)) is about 0.10 mg/kg per day, about 0.15 mg/kg per day, about 0.20 mg/kg per day, about 0.25 mg/kg per day, about 0.30 mg/kg per day, about 0.35 mg/kg per day, about 0.40 mg/kg per day, about 0.45 mg/kg per day, about 0.50 mg/kg per day, about 0.55 mg/kg per day, about 0.60 mg/kg per day, about 0.65 mg/kg per day, about 0.70 mg/kg per day, about 0.75 mg/kg per day, about 0.80 mg/kg per day, about 0.85 mg/kg per day, about 0.90 mg/kg per day, about 0.95 mg/kg per day, or about 1.00 mg/kg per day.

In some embodiments, the therapeutically effective amount of a compound of Formula (I) (preferably, the compound of Formula (I-g)) is about 1.05 mg/kg per day, about 1.10 mg/kg per day, about 1.15 mg/kg per day, about 1.20 mg/kg per day, about 1.25 mg/kg per day, about 1.30 mg/kg per day, about 1.35 mg/kg per day, about 1.40 mg/kg per day, about 1.45 mg/kg per day, about 1.50 mg/kg per day, about 1.55 mg/kg per day, about 1.60 mg/kg per day, about 1.65 mg/kg per day, about 1.70 mg/kg per day, about 1.75 mg/kg per day, about 1.80 mg/kg per day, about 1.85 mg/kg per day, about 1.90 mg/kg per day, about 1.95 mg/kg per day, or about 2.00 mg/kg per day.

In some embodiments, the therapeutically effective amount of a compound of Formula (I) (preferably, the compound of Formula (I-g)) is about 2 mg/kg per day, about 2.5 mg/kg per day, about 3 mg/kg per day, about 3.5 mg/kg per day, about 4 mg/kg per day, about 4.5 mg/kg per day, about 5 mg/kg per day, about 5.5 mg/kg per day, about 6 mg/kg per day, about 6.5 mg/kg per day, about 7 mg/kg per day, about 7.5 mg/kg per day, about 8.0 mg/kg per day, about 8.5 mg/kg per day, about 9.0 mg/kg per day, about 9.5 mg/kg per day, or about 10 mg/kg per day.

In some embodiments, the therapeutically effective amount of a compound of Formula (I) (preferably, the compound of Formula (I-g)) is administered to the subject once daily. In some embodiments, this daily dose of a compound of Formula (I) (preferably, the compound of Formula (I-g)) may be administered to the subject all at once. In some embodiments, this daily dose of a compound of Formula (I) (preferably, the compound of Formula (I-g)) may be administered to the subject in two portions (a divided dose). In some embodiments, this daily dose of a compound of Formula (I) (preferably, the compound of Formula (I-g)) may be administered to the subject in three portions. In some embodiments, this daily dose of a compound of Formula (I) (preferably, the compound of Formula (I-g)) may be administered to the subject in four portions. In some embodiments, this daily dose of a compound of Formula (I) (preferably, the compound of Formula (I-g)) may be administered to the subject in five or more portions. In some embodiments, these portions are administered to the subject at regular intervals throughout the day, for example, every 12 hours, every 8 hours, every 6 hours, every 5 hours, every 4 hours, 3 hours, 2 hours, 1 hour, etc.

In some embodiments, the therapeutically effective amount of a compound of Formula (I) (preferably, the compound of Formula (I-g)) is administered to the subject twice daily. In some embodiments, this twice daily dose of a compound of Formula (I) (preferably, the compound of Formula (I-g)) may be administered to the subject all at once. In some embodiments, this twice daily dose of a compound of Formula (I) (preferably, the compound of Formula (I-g)) may be administered to the subject in two portions (a divided dose). In some embodiments, this twice daily dose of a compound of Formula (I) (preferably, the compound of Formula (I-g)) may be administered to the subject in three portions. In some embodiments, this twice daily dose of a compound of Formula (I) (preferably, the compound of Formula (I-g)) may be administered to the subject in four portions. In some embodiments, this twice daily dose of a compound of Formula (I) (preferably, the compound of Formula (I-g)) may be administered to the subject in five or more portions. In some embodiments, these portions are administered to the subject at regular intervals throughout the day, for example, every 12 hours, every 8 hours, every 6 hours, every 5 hours, every 4 hours, 3 hours, 2 hours, 1 hour, etc.

In some embodiments, the therapeutically effective amount of the compound of Formula (I) (preferably, the compound of Formula (I-g)) results in a mean day 15 AUC₀₋₂₄ of greater than about 3,500 ng*hr/mL, about 3,550 ng*hr/mL, about 3,600 ng*hr/mL, about 3,650 ng*hr/mL, about 3,700 ng*hr/mL, about 3,750 ng*hr/mL, about 3,800 ng*hr/mL, about 3,850 ng*hr/mL, about 3,900 ng*hr/mL, about 3,950 ng*hr/mL, about 4,000 ng*hr/mL, 4,050 ng*hr/mL, about 4,100 ng*hr/mL, about 4,150 ng*hr/mL, about 4,200 ng*hr/mL, 4,250 ng*hr/mL, about 4,300 ng*hr/mL, about 4,350 ng*hr/mL, about 4,400 ng*hr/mL, about 4,450 ng*hr/mL, about 4,500 ng*hr/mL, about 4,550 ng*hr/mL, about 4,600 ng*hr/mL, about 4,650 ng*hr/mL, about 4,700 ng*hr/mL, about 4,750 ng*hr/mL, about 4,800 ng*hr/mL, about 4,850 ng*hr/mL, about 4,900 ng*hr/mL, about 4,950 ng*hr/mL, about 5,000 ng*hr/mL, 5,050 ng*hr/mL, about 5,100 ng*hr/mL, about 5,150 ng*hr/mL, about 5,200 ng*hr/mL, about 5,250 ng*hr/mL, about 5,300 ng*hr/mL, about 5,350 ng*hr/mL, about 5,400 ng*hr/mL, about 5,450 ng*hr/mL, about 5,500 ng*hr/mL, about 5,550 ng*hr/mL, about 5,600 ng*hr/mL, about 5,650 ng*hr/mL, about 5,700 ng*hr/mL, about 5,750 ng*hr/mL, about 5,800 ng*hr/mL, about 5,850 ng*hr/mL, about 5,900 ng*hr/mL, 5,950 ng*hr/mL, or about 6,000 ng*hr/mL, 6,050 ng*hr/mL, about 6,100 ng*hr/mL, about 6,150 ng*hr/mL, about 6,200 ng*hr/mL, about 6,250 ng*hr/mL, about 6,300 ng*hr/mL, about 6,350 ng*hr/mL, about 6,400 ng*hr/mL, about 6,450 ng*hr/mL, about 6,500 ng*hr/mL, about 6,550 ng*hr/mL, about 6,600 ng*hr/mL, about 6,650 ng*hr/mL, about 6,700 ng*hr/mL, about 6,750 ng*hr/mL, about 6,800 ng*hr/mL, about 6,850 ng*hr/mL, about 6,900 ng*hr/mL, 6,950 ng*hr/mL, or about 7,000 ng*hr/mL, 7,050 ng*hr/mL, about 7,100 ng*hr/mL, about 7,150 ng*hr/mL, about 7,200 ng*hr/mL, about 7,250 ng*hr/mL, about 7,300 ng*hr/mL, about 7,350 ng*hr/mL, about 7,400 ng*hr/mL, about 7,450 ng*hr/mL, about 7,500 ng*hr/mL, about 7,550 ng*hr/mL, about 7,600 ng*hr/mL, about 7,650 ng*hr/mL, about 7,700 ng*hr/mL, about 7,750 ng*hr/mL, about 7,800 ng*hr/mL, about 7,850 ng*hr/mL, about 7,900 ng*hr/mL, 7,950 ng*hr/mL, or about 8,000 ng*hr/mL, 8,050 ng*hr/mL, about 8,100 ng*hr/mL, about 8,150 ng*hr/mL, about 8,200 ng*hr/mL, about 8,250 ng*hr/mL, about 8,300 ng*hr/mL, about 8,350 ng*hr/mL, about 8,400 ng*hr/mL, about 8,450 ng*hr/mL, about 8,500 ng*hr/mL, about 8,550 ng*hr/mL, about 8,600 ng*hr/mL, about 8,650 ng*hr/mL, about 8,700 ng*hr/mL, about 8,750 ng*hr/mL, about 8,800 ng*hr/mL, about 8,850 ng*hr/mL, about 8,900 ng*hr/mL, 8,950 ng*hr/mL, or about 9,000 ng*hr/mL.

In some embodiments, the therapeutically effective amount of the compound of Formula (I) (preferably, the compound of Formula (I-g)) results in a mean day 15 Cmax of greater than about 250 ng/mL, about 255 ng/mL, about 260 ng/mL, about 265 ng/mL, about 270 ng/mL, about 275 ng/mL, about 280 ng/mL, about 285 ng/mL, about 290 ng/mL, about 295 ng/mL, about 300 ng/mL, about 305 ng/mL, about 310 ng/mL, about 315 ng/mL, about 320 ng/mL, about 325 ng/mL, about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, about 380 ng/mL, about 385 ng/mL, about 390 ng/mL, about 395 ng/mL, about 400 ng/mL, about 405 ng/mL, about 410 ng/mL, about 415 ng/mL, about 420 ng/mL, about 425 ng/mL, about 430 ng/mL, about 435 ng/mL, about 440 ng/mL, about 445 ng/mL, about 450 ng/mL, about 455 ng/mL, about 460 ng/mL, about 465 ng/mL, about 470 ng/mL, about 475 ng/mL, about 480 ng/mL, about 485 ng/mL, about 490 ng/mL, about 495 ng/mL, or about 500 ng/mL.

The therapeutically effective amount of a compound of Formula (I) (preferably, the compound of Formula (I-g)) can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the subject, and the route of administration.

Dosage and administration are adjusted to provide sufficient levels of a compound of Formula (I) (preferably, the compound of Formula (I-g)) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In some embodiments, for the methods of treating prostate cancer with the combination of a compound of Formula (I) (preferably, the compound of Formula (I-g)) and abiraterone, a pharmaceutically acceptable salt of abiraterone, abiraterone acetate, or a pharmaceutically acceptable salt of abiraterone acetate, the therapeutically effective amount of a compound of Formula (I) (preferably, the compound of Formula (I-g)) is described herein, and the therapeutically effective amount of abiraterone, a pharmaceutically acceptable salt of abiraterone, abiraterone acetate, or a pharmaceutically acceptable salt of abiraterone acetate is about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer.

In some embodiments, for the methods of treating prostate cancer with the combination of a compound of Formula (I) (preferably, the compound of Formula (I-g)) and abiraterone acetate, the therapeutically effective amount of a compound of Formula (I) (preferably, the compound of Formula (I-g)) is described herein, and the therapeutically effective amount of abiraterone acetate is about 250 mg, about 500 mg, about 750 mg, about 1,000 mg, about 1,250 mg, or about 1,500 mg administered once daily.

In some embodiments, for the methods of treating prostate cancer with the combination of a compound of Formula (I) (preferably, the compound of Formula (I-g)) and abiraterone acetate, the therapeutically effective amount of a compound of Formula (I) (preferably, the compound of Formula (I-g)) is described herein, and the therapeutically effective amount of abiraterone acetate is 1,000 mg administered orally once daily for one, two, three, four, five, six, seven, eight, nine, ten, eleven, twelve, thirteen, fourteen, fifteen, thirty, or more consecutive days, in single or divided doses. In some embodiments, the abiraterone acetate is administered in combination with 5 mg of prednisone or prednisolone administered orally, twice daily.

In some embodiments, the combination of the compound of Formula (I) (preferably, the compound of Formula (I-g)) and abiraterone acetate is administered to the subject in need thereof in the fasted state. In some embodiments, the subject does not eat for at least two hours before, and at least one hour after, the administration of the combination of the compound of Formula (I) (preferably, the compound of Formula (I-g)) and abiraterone acetate.

In some embodiments, the combination of the compound of Formula (I) (preferably, the compound of Formula (I-g)) and abiraterone acetate is administered to the subject in need thereof in the fed state. In some embodiments, the subject has eaten within any of 4, 3, 2, 1, or 0.5 hours prior to administration of a compound of Formula (I) (preferably, the compound of Formula (I-g)).

In some embodiments, the compound of Formula (I) (preferably, the compound of Formula (I-g)) and abiraterone acetate are administered to the subject simultaneously.

In some embodiments, the compound of Formula (I) (preferably, the compound of Formula (I-g)) and abiraterone, a pharmaceutically acceptable salt of abiraterone, abiraterone acetate, or a pharmaceutically acceptable salt of abiraterone acetate are administered to the subject simultaneously in separate formulations (e.g., in separate tablets). In some embodiments, the compound of Formula (I) (preferably, the compound of Formula (I-g)) and abiraterone, a pharmaceutically acceptable salt of abiraterone, abiraterone acetate, or a pharmaceutically acceptable salt of abiraterone acetate are administered to the subject simultaneously in the same formulation (e.g., in a single tablet).

In some embodiments, the compound of Formula (I) (preferably, the compound of Formula (I-g)) and abiraterone acetate are administered to the subject sequentially.

In some embodiments, the compound of Formula (I) (preferably, the compound of Formula (I-g)) and abiraterone acetate are administered to the subject in temporal proximity.

In some embodiments, “temporal proximity” means that administration of compound of Formula (I) (preferably, the compound of Formula (I-g)) occurs within a time period before or after the administration of abiraterone acetate, such that the therapeutic effect of the compound of Formula (I) (preferably, the compound of Formula (I-g)) overlaps with the therapeutic effect of abiraterone acetate. In some embodiments, the therapeutic effect of the compound of Formula (I) (preferably, the compound of Formula (I-g)) completely overlaps with the therapeutic effect of abiraterone acetate. In some embodiments, “temporal proximity” means that administration of the compound of Formula (I) (preferably, the compound of Formula (I-g)) occurs within a time period before or after the administration of abiraterone acetate, such that there is a synergistic effect between the compound of Formula (I) (preferably, the compound of Formula (I-g)) and abiraterone acetate.

“Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

### Pharmaceutical Compositions

In some embodiments, a compound of Formula (I) (preferably, the compound of Formula (I-g)) is formulated for oral administration. For example, in some embodiments, a compound of Formula (I) (preferably, the compound of Formula (I-g)) is formulated as a tablet that comprises zero, one, two, or more of each of the following: emulsifier; surfactant, binder; disintegrant, glidant; and lubricant.

In some embodiments, the emulsifier is hypromellose.

In some embodiments, the surfactant is vitamin E polyethylene glycol succinate.

In some embodiments, the binder (also referred to herein as a filler) is selected from the group consisting of microcrystalline cellulose, lactose monohydrate, sucrose, glucose, and sorbitol.

In some embodiments, the disintegrant is croscarmellose sodium.

In some embodiments, the glidant refers to a substance used to promote powder flow by reducing interparticle cohesion. In some embodiments, in the dosage forms of the disclosure, the glidant is selected from the group consisting of silicon dioxide, silica colloidal anhydrous, starch, and talc.

In some embodiments, the lubricant refers to a substance that prevents ingredients from sticking and/or clumping together in the machines used in preparation of the dosage forms of the disclosure. In some embodiments, in the dosage forms of the disclosure, the lubricant is selected from the group consisting of magnesium stearate, sodium stearyl fumarate, stearic acid, and vegetable stearin.

The pharmaceutical compositions containing a compound of Formula (I) (preferably, the compound of Formula (I-g)) may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of a compound of Formula (I) (preferably, the compound of Formula (I-g)) into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a compound of Formula (I) (preferably, the compound of Formula (I-g)) in the required amount in an appropriate solvent with one or a combination of ingredients enumerated above, as required, followed by filtered sterilization. Generally, dispersions are prepared by incorporating the active agent or compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, a compound of Formula (I) (preferably, the compound of Formula (I-g)) can be incorporated with excipients and used in the form of tablets, troches, or capsules. Oral compositions can also be prepared using a fluid carrier for use as a mouthwash, wherein the agent or compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, sodium starch glycolate (Primojel®), or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the agents or compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents or compounds are formulated into ointments, salves, gels, or creams as generally known in the art.

In one aspect, a compound of Formula (I) (preferably, the compound of Formula (I-g)) is prepared with pharmaceutically acceptable carriers that will protect the agent or compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent or compound calculated to produce the desired therapeutic effect in association with the required pharmaceutical carrier. The specification for the dosage unit forms of the application are dictated by and directly dependent on the unique characteristics of a compound of Formula (I) (preferably, the compound of Formula (I-g)) and the particular therapeutic effect to be achieved.

The pharmaceutical compositions can be included in a container, pack, or dispenser together with instructions for administration.

Illustrative modes of administration for a compound of Formula (I) (preferably, the compound of Formula (I-g)) includes systemic or local administration such as oral, nasal, parenteral, transdermal, subcutaneous, vaginal, buccal, rectal or topical administration modes. In some embodiments, the compound of Formula (I) (preferably, the compound of Formula (I-g)), or a pharmaceutically acceptable salt or hydrate thereof, is administered orally. In some embodiments, the compound of Formula (I) (preferably, the compound of Formula (I-g)) is administered as a tablet, capsule, caplet, solution, suspension, syrup, granule, bead, powder, or pellet.

Illustrative pharmaceutical compositions are tablets and gelatin capsules comprising a salt of compound of Formula (I) (preferably, the compound of Formula (I-g)) and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, and/or PEG200.

For preparing pharmaceutical compositions from a compound of Formula (I) (preferably, the compound of Formula (I-g)), or a salt or hydrate thereof, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed salt is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Pharmaceutical compositions can be prepared according to conventional mixing, granulating or coating methods, respectively, and the present pharmaceutical compositions can contain from about 0.1% to about 99%, from about 5% to about 90%, or from about 1% to about 20% of the disclosed salt by weight or volume.

All amounts of any component of an oral dosage form described herein, e.g., a tablet, that are indicated based on % w/w refer to the total weight of the oral dosage form, unless otherwise indicated.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1—In Vitro Studies with Compound (I-g)

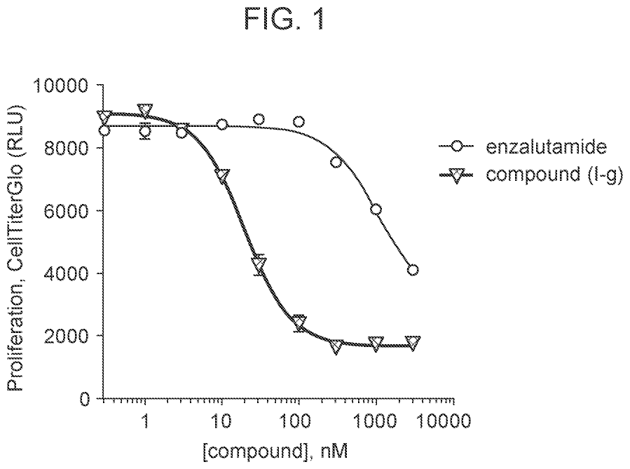

Compound (I-g) was shown to degrade 95% to 98% of androgen receptors (AR) in multiple cells lines typically used in prostate cancer research, including, for example, VCaP cells. (DC₅₀ in VCaP for Compound (I-g) is 1 nM.) Near-maximal degradation was observed within 4 hours of administration of Compound (I-g). Compound (I-g) inhibits VCaP proliferation about 60 times more potently than enzalutamide. (FIG. 1.)

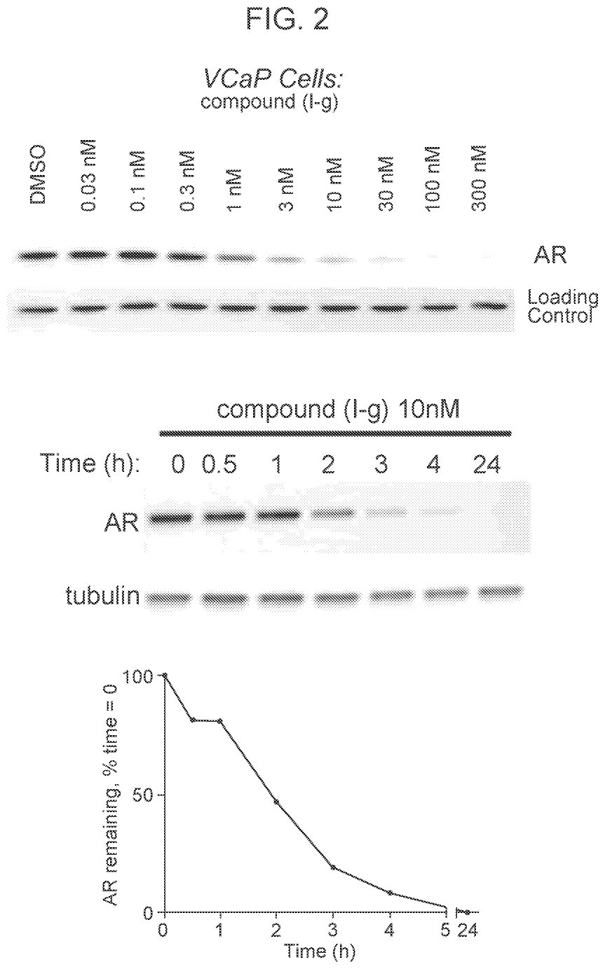

FIG. 2 shows the reduction of AR in VCaP tumor cells in response to treatment with Compound (I-g) at concentrations of 0.03 nM, 0.1 nM, 0.3 nM, 1 nM, 3 nM, 10 nM, 30 nM, 100 nM, and 300 nM.

### Example 2—In Vivo Studies with Animals and Assessment of the Preclinical Efficacious Exposure Range for Compound (I-g)

Preclinical animal studies were performed with Compound (I-g) in VCaP xenograft animal models. VCaP was derived from a vertebral metastatic growth of a prostate carcinoma. It is a desirable cell line for in vivo studies as it exhibits many of the characteristics of clinical prostate carcinoma. VCaP is also a useful model to study AR resistance as it expresses AR splice variants that have been shown to drive resistance to AR antagonists. (European Urology. 2018 April; 73(4):572-582.)

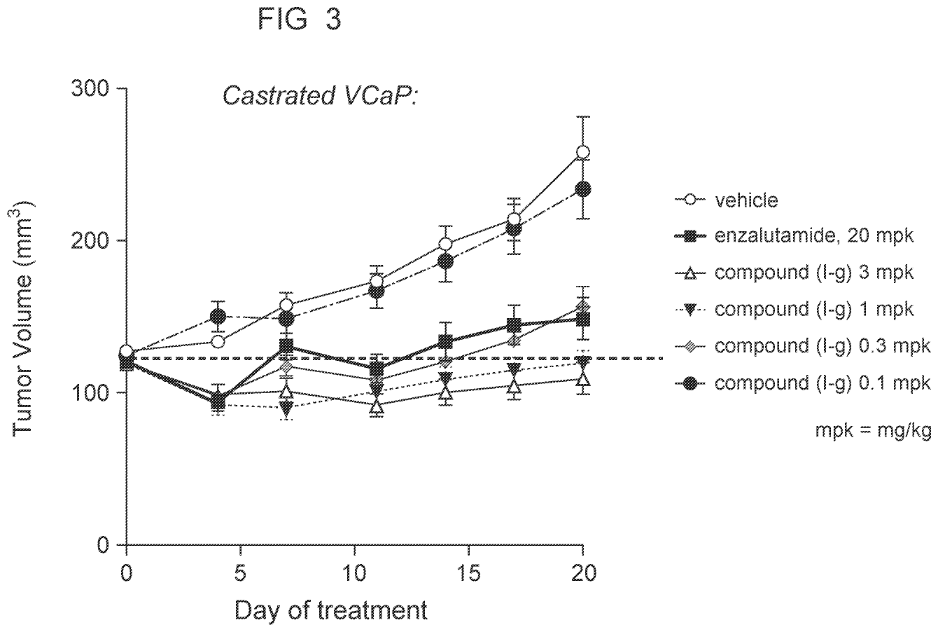

Oral, once daily administration of Compound (I-g) at doses of 0.1 mg/kg (mpk), 0.3 mg/kg, 1 mg/kg, and 3 mg/kg were performed in a castrated VCaP xenograft model (FIG. 3). Enzalutamide (20 mg/kg) and vehicle were also used as control groups.

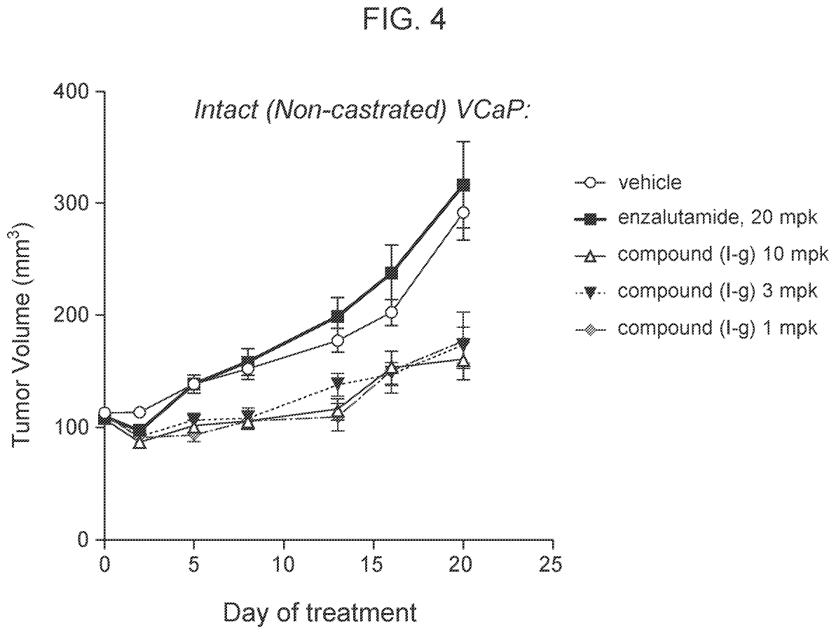

Oral, once daily administration of Compound (I-g) at doses of 1 mg/kg, 3 mg/kg, 10 mg/kg were performed in an intact (non-castrated) VCaP xenograft model (FIG. 4). Enzalutamide (20 mg/kg) and vehicle were also used as control groups.

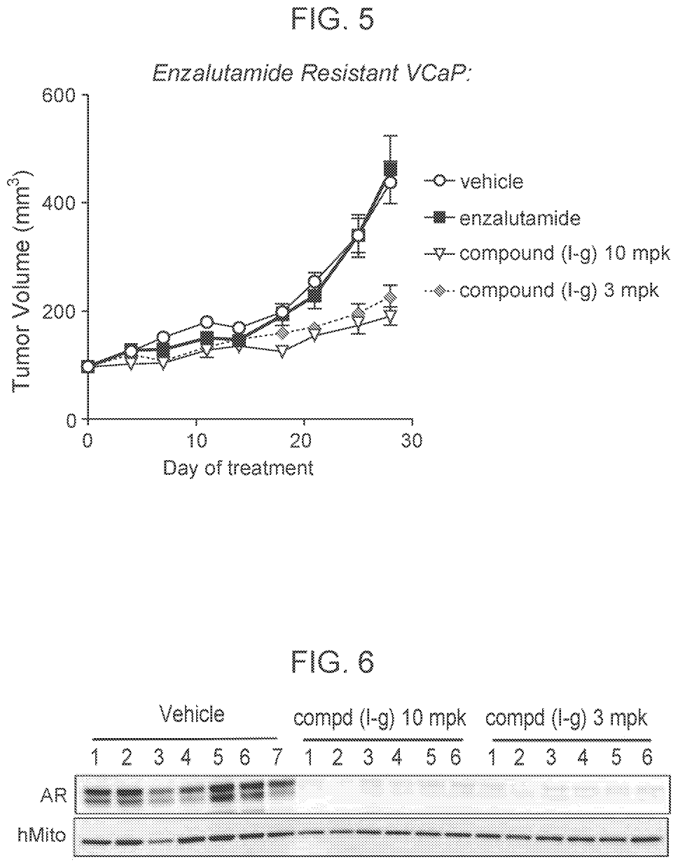

Oral, once daily administration of Compound (I-g) at doses of 3 mg/kg and 10 mg/kg were performed in an enzalutamide resistant VCaP xenograft model (FIG. 5). Enzalutamide (20 mg/kg) and vehicle were also used as control groups.

The pharmacokinetic results of oral, once daily administration of Compound (I-g) at doses of 1 mg/kg and 3 mg/kg are shown below in Table 1. A dose of 1 mg/kg of Compound (I-g) is the lowest dose that is superior to enzalutamide in a VCaP xenograft. A 3 mg/kg dose of Compound (I-g) was the lowest efficacious dose in an enzalutamide-resistant VCaP model (tumor growth inhibition of 70% compared to a control group).

FIG. 6 shows the reduction of AR in enzalutamide-resistant VCaP tumors in response to dosing with Compound (I-g) at 10 mg/kg and 3 mg/kg (oral, once daily).

TABLE 1

| Dose | Mean AUC₀₋₂₄ | Mean Cₘₐₓ |
|---|---|---|
| (oral, once daily) | (ng*hr/mL) ^{†} | (ng/mL) ^{‡} |
| 1 mg/kg | 3628 | 224 |
| 3 mg/kg | 8106 | 507 |

Values represent total drug concentrations
^{†} AUC or Area Under the Curve is a measurement of total exposure
^{‡} Cₘₐₓ is a measurement of peak concentration during the dosing period

### Example 3—In Vivo Animal Studies with Compound (I-g) and Abiraterone

The combination of Compound (I-g) and abiraterone attenuated tumor growth more significantly than either agent alone in castrated VCaP xenografts.

### Example 4—Toxicology Studies

Animals were orally administered compound (I-g) once daily for 28 days, followed by a 14-day recovery for high-dose animals.

In dogs, once daily, oral doses of 3 mg/kg, 10 mg/kg, or 30 mg/kg of Compound (I-g) were administered. It was determined that the 30 mg/kg dose exceeded the maximum tolerated dose. Gastrointestinal alterations were observed at all dose levels (including vehicle alone). Reversible liver function enzyme elevation, which is considered non-adverse, was observed in some mid- and high-dose animals. Male animals exhibited decreased prostate weights, which may be attributable to the pharmacology of Compound (I-g).

In rats, males were administered once daily, oral doses of Compound (I-g) at doses of 20 mg/kg, 60 mg/kg, or 120 mg/kg. Female rats were administered once daily, oral doses of Compound (I-g) at doses of 20 mg/kg, 40 mg/kg, or 120 mg/kg.

Overall, Compound (I-g) was well tolerated at all doses, with the exception of the 80 mg/kg female cohort. These rats lost body weight and consumed less food. All of the findings in male high-dose rats were fully reversible (liver hypertrophy, femur physis thickening). Male rats also exhibited decreased prostate weights, which may be attributable to the pharmacology of Compound (I-g).

### Example 5—Phase I Clinical Trial Study Design with Compound (I-g)

A Phase I Clinical Trial with Compound (I-g) was undertaken. A traditional 3+3 dose escalation design was implemented. Starting dose of Compound (I-g) was 35 mg administered orally, once daily with food. Dose increases were dependent upon toxicities.

The key criteria for this trial were: men with metastatic, castrate-resistant prostate cancer (mCRPC); at least two prior systemic therapies, at least one of which was abiraterone or enzalutamide; and disease progression on most recent therapy (for example, rising PSA or two or more new lesions upon bone scan).

The key objectives for this trial were obtaining the maximum tolerated dose of Compound (I-g) and the recommended Phase II trial dose. Additional objectives included assessing overall safety of Compound (I-g), pharmacokinetics, anti-tumor activity (for example, PSA, RECIST), and biomarkers, including, for example, AR degradation in CTCs and pre- vs. post-treatment biopsies (when available); AR (and other) gene mutations, amplifications in ctDNA; and AR-V7 in CTCs.

### Example 6—Phase I Pharmacokinetic Data—Oral Administration of Compound (I-g)

In a Phase I clinical trial, Compound (I-g) was administered orally at a dose of 35 mg/day, 70 mg/day, and 140 mg/day. It was observed that treatment with 140 mg/day dose of Compound (I-g) enters the preclinical efficacious range associated with tumor growth inhibition.

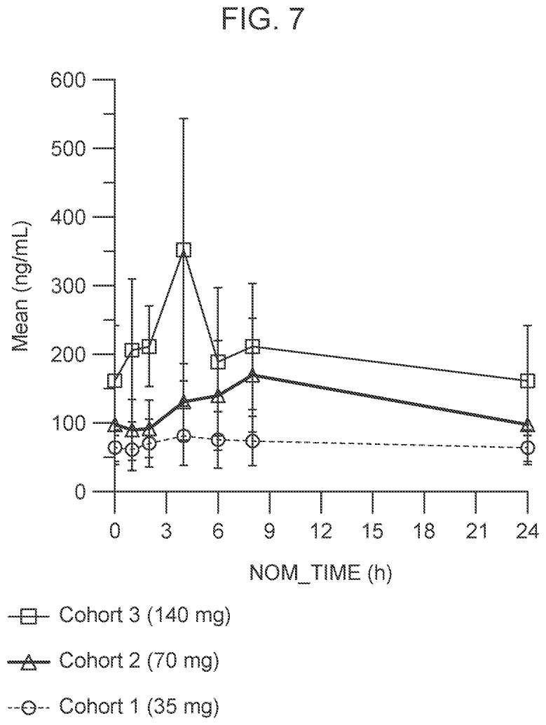

The initial pharmacokinetic results are shown below in Table 2, as well as in FIG. 7, which provides a representation of the mean concentrations of Compound (I-g) over a 24 hour time period after dosing on day 15 for all three tested doses (35 mg/day, 70 mg/day, and 140 mg/day).

TABLE 2

| Dose | Mean Day 1 | | Mean Day 15 | |
|---|---|---|---|---|
| (oral, once | AUC₀₋₂₄ | Mean Day 1 | AUC₀₋₂₄ | Mean Day 15 |
| daily) | (ng*hr/mL) | Cₘₐₓ (ng/mL) | (ng*hr/mL)^{a} | Cₘₐₓ (ng/mL) |
| 35 mg | 160.5 | 11.1 | 1701 | 83 |
| 70 mg | 300 | 19.6 | 2538 | 141 |
| 140 mg | 865 | 54 | 5023 | 353 |

^{a}Day 15 AUCs calculated using imputed 24 hours values.

### Example 7—Phase I Dose Escalation Studies with Compound (I-g)

Compound (I-g) was administered orally to human subjects (n=22) at doses of 35 mg/day, 70 mg/day, 140 mg/day, and 280 mg/day.

In the 35 mg/day cohort (n=3), no dose limiting toxicity was observed and no adverse events at grades 2, 3, or 4 were observed.

In the 70 mg/day cohort (n=4), no dose limiting toxicity was observed. One patient experienced grade 2 adverse events (diarrhea, fatigue, vomiting). One patient experienced a grade 3 adverse event (anemia) that was unrelated to the administration of compound (I-g).

In the 140 mg/day cohort (n=8), no dose limiting toxicity was observed. 50% of the patients experienced grade 2 adverse events and 1 patient experienced a grade 3 adverse event (decreased lymphocyte count). These results do not include one patient in this cohort group who was determined to be non-evaluable and treatment was discontinued on day 1.

In the 280 mg/day cohort (n=7), one patient experienced dose-limiting toxicity and renal failure, and 5 of the patients experienced grade 2 or less adverse events.

### Example 8—Evaluation of Best Percent Change of Plasma PSA from Pre-Treatment Levels in Patients with mCRPC and Subsequent Evaluation of Biomarker Status After Oral Administration of Compound (I-g)

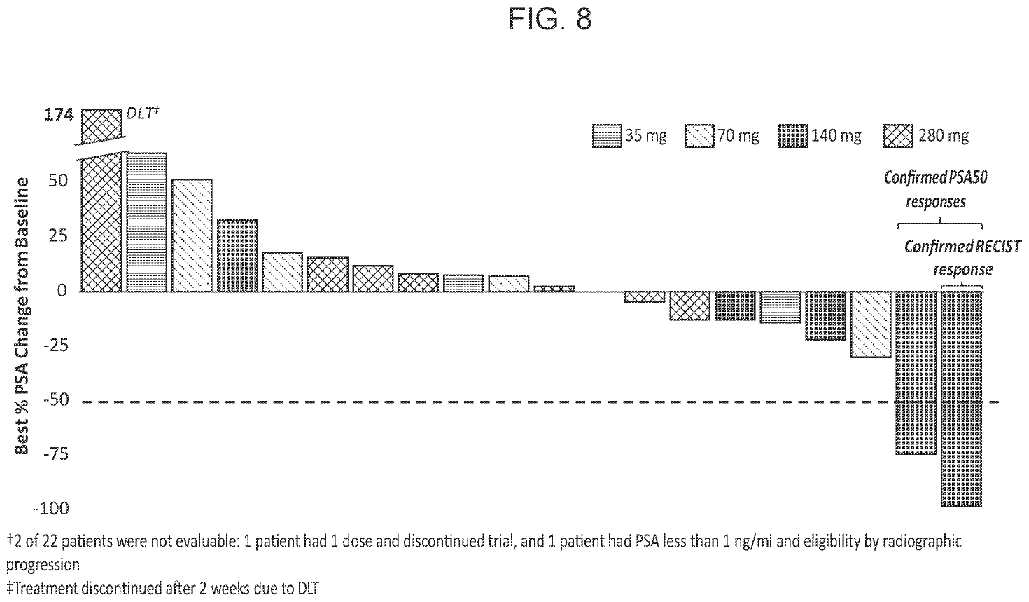

Twenty patients were administered Compound (I-g) orally at doses of 35 mg/day, 70 mg/day, 140 mg/day, or 280 mg/day. The best percent change in plasma PSA from pre-treatment levels for each of the twenty patients is provided in FIG. 8. Patient 19 (second bar from right) and Patient 20 (rightmost bar) had at least a 50% reduction in PSA after treatment with Compound (I-g).

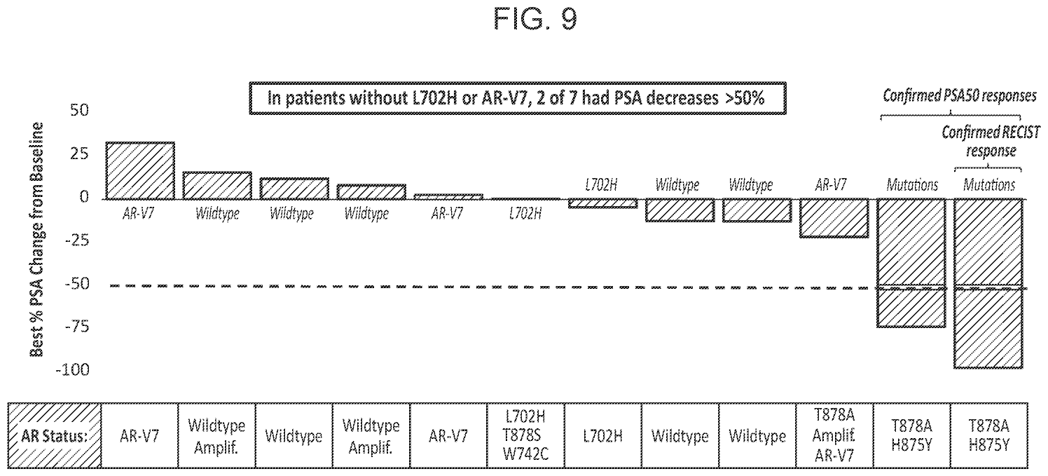

The AR biomarker status of twelve patients who were administered Compound (I-g) orally at a dose greater than or equal to 140 mg/day was evaluated. FIG. 9 shows the AR biomarker status of these 12 patients along with their best percent change in plasma PSA levels. Patients with different AR biomarker status had different responses to treatment with Compound (I-g). For instance, Patient 19 (second bar from right) and Patient 20 (rightmost bar), who both had T878A and H875Y AR mutations, were the only patients in this study who had at least a 50% reduction in PSA after treatment.

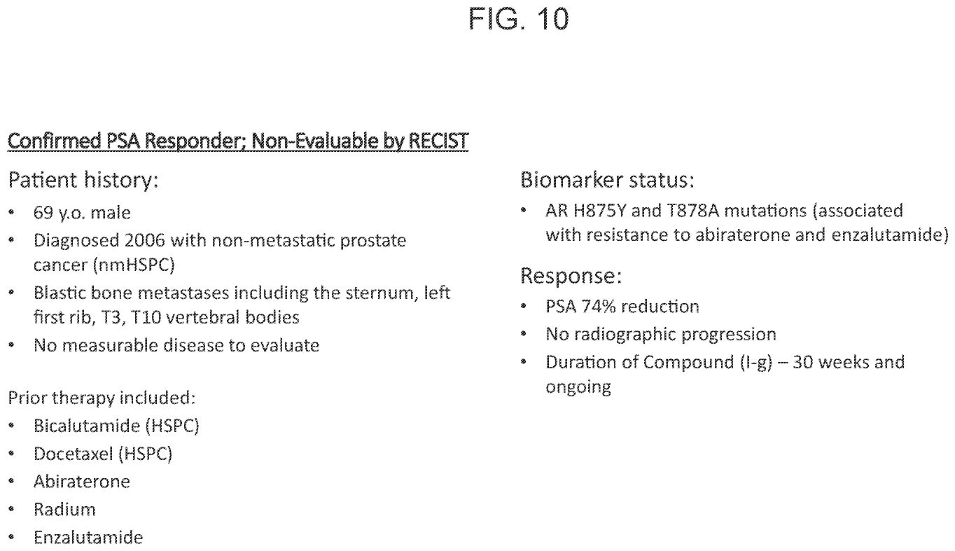

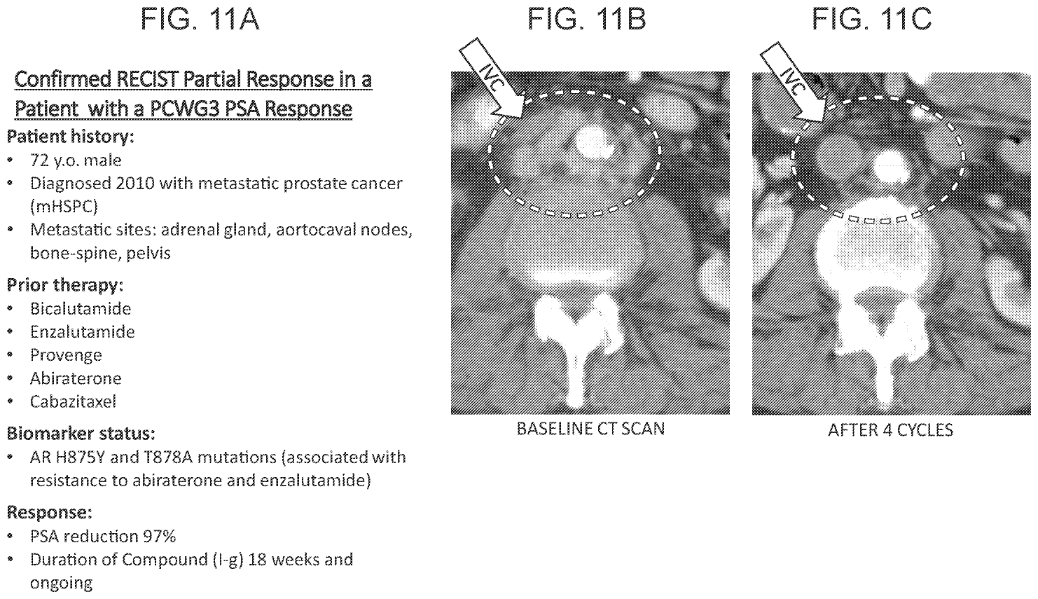

The key features of Patients 19 and 20 are summarized in FIG. 10 and FIG. 11A, respectively. FIG. 11B shows a CT scan of Patient 20's tumor prior to treatment with Compound (I-g). FIG. 11C shows a CT scan of Patient 20's tumor after 4 cycles, showing the RECIST response.

### Example 9—Further Pharmacokinetic Data—Oral Administration of Compound (I-g)

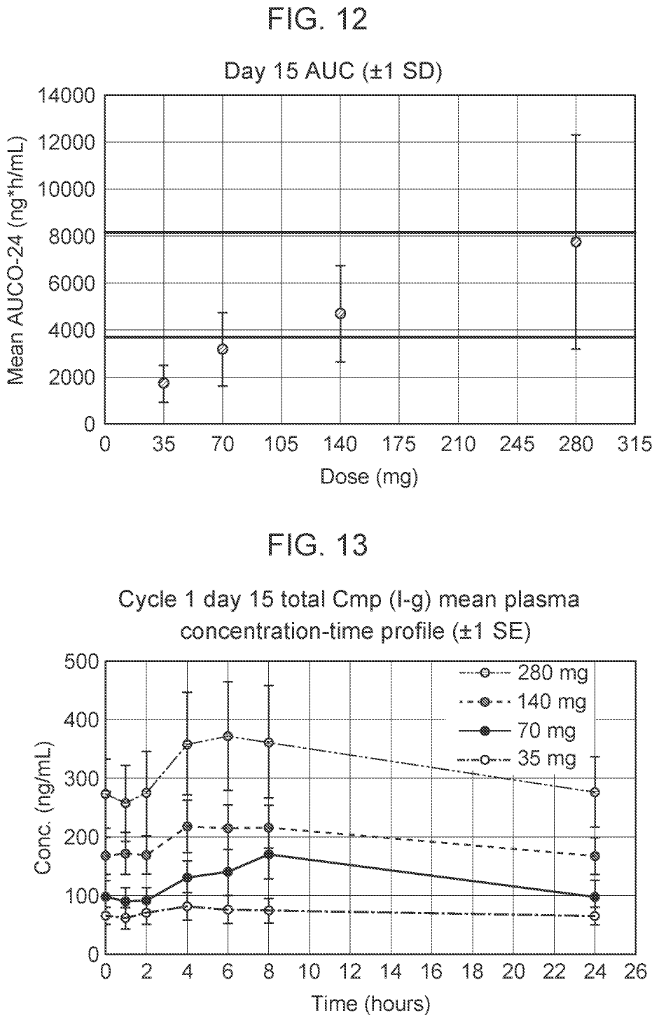

Compound (I-g) was administered orally at a dose of 35 mg/day, 70 mg/day, 140 mg/day, and 280 mg/day. It was observed that treatment with 140 mg/day and 280 mg/day dose of Compound (I-g) enters the preclinical efficacious range associated with tumor growth inhibition. (FIG. 12.) The mean plasma concentrations of Compound (I-g) over a 24 hour time period after dosing on day 15 for all four tested doses (35 mg/day, 70 mg/day, 140 mg/day, and 280 mg/day) are provided in FIG. 13.

### Example 10—Efficacy of Compound (I-g) and Abiraterone

The efficacy of Compound (I-g) and a standard of care agent, abiraterone (Zytiga®) was assessed in a 3-stage study using castrated mice bearing human VCaP (Vertebral-Cancer of the Prostate) tumor xenografts. The first stage of the study evaluated the efficacy of single agents and the combination regimen (“Stage I”). In the second stage of the study (“Stage II”), mice in the abiraterone arm were continuously dosed until the tumors became abiraterone resistant. In the third stage of the study (“Stage III”), the efficacy of Compound (I-g) was evaluated in the abiraterone-resistant tumors.

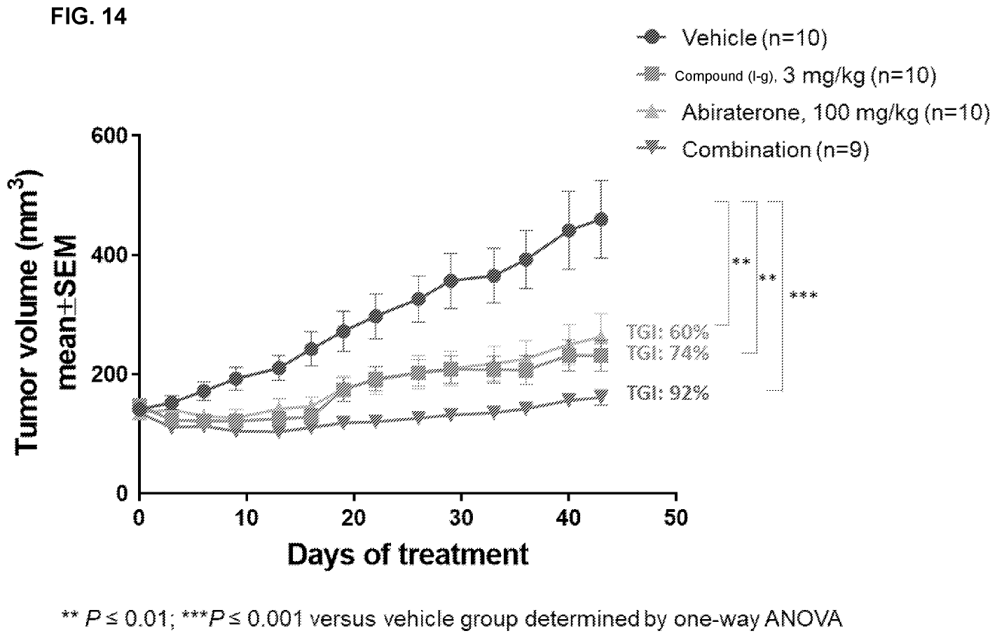

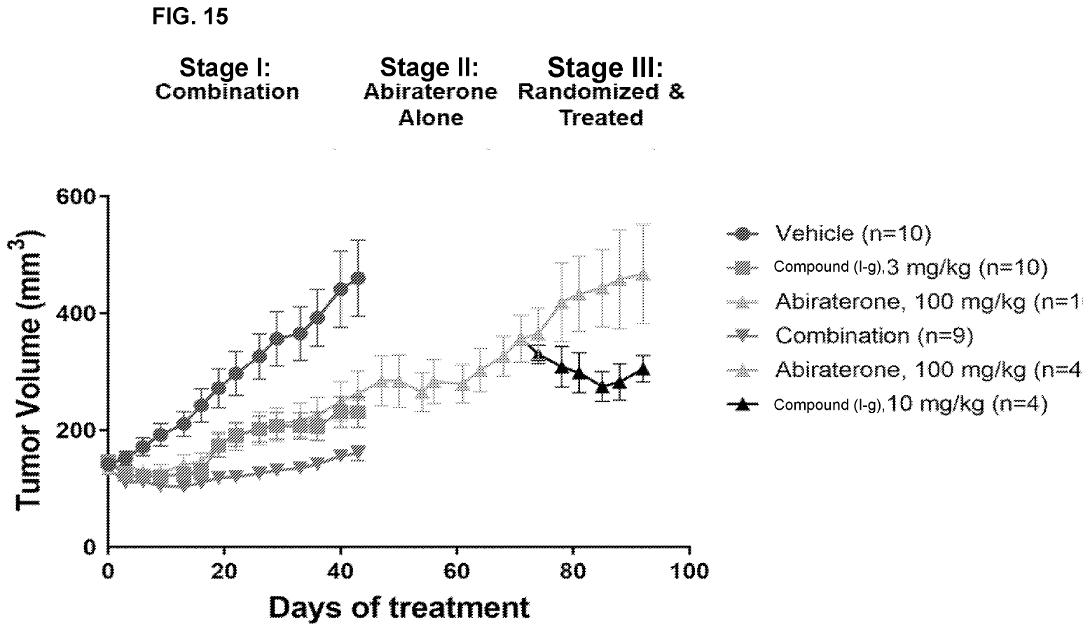

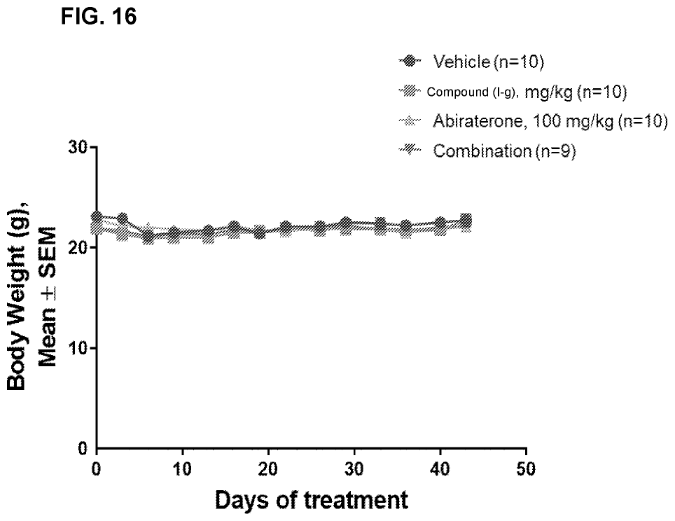

In Stage I, surgically castrated CB17/scid male mice with subcutaneously implanted VCaP tumors in the dorsal flank were treated with single agent Compound (I-g) (3 mg/kg) or abiraterone (100 mg/kg), or a combination of both agents by oral gavage once daily. As single agents, Compound (I-g) and abiraterone demonstrated significant tumor growth inhibition (TGI), 74% and 60%, respectively. (FIG. 14, FIG. 15, Stage III, and Table 3). During the treatment period, no significant change in body weight was observed (FIG. 16).

In Stage III, animals with abiraterone-resistant tumors generated in Stage II were randomized when those tumors reached the size of the tumors in the vehicle arm at the end of the Stage I portion of the study. The animals were then treated with either 10 mg/kg Compound (I-g) or 100 mg/kg abiraterone by oral gavage once daily. As shown in FIG. 15 (Stage III) and Table 3, growth inhibition was observed in Compound (I-g) treated animals bearing abiraterone-resistant tumors.

TABLE 3
Tumor growth inhibition

| | | Compound | | | | Compound |
|---|---|---|---|---|---|---|
| | | (I-g), | Abiraterone, | | Abiraterone, | (I-g), |
| | Vehicle, | 3 mg/kg, | 100 mg/kg, | Combination, | 100 mg/kg, | 10 mg/kg, |
| | n = 10 | n = 10 | n = 10 | n = 9 | n = 4 | n = 4 |
| | (Stage I) | (Stage I) | (Stage I) | (Stage I) | (Stage III) | (Stage III) |
| Tumor | 141 ± 10 | 147 ± 8\grave{ } | 135 ± 10 | 135 ± 8 | 365 ± 44 | 330 ± 15 |
| volume, Day | | | | | | |
| 0 (mm³) | | | | | | |
| Tumor | 460 ± 65 | 231 ± 26 | 262 ± 39 | 161 ± 13 | 467 ± 85 | 305 ± 23 |
| volume, Day | | | | | | |
| 43 (Stage I) | | | | | | |
| or Day 92 | | | | | | |
| (Stage III) | | | | | | |
| (mm³) | | | | | | |
| TGI | — | 74 | 60 | 92 | — | 124 |
| (%) vs | | | | | | |
| vehicle | | | | | | |
| (Stage I) or | | | | | | |
| vs | | | | | | |
| Abiraterone | | | | | | |
| (Stage III) | | | | | | |

Tumor volumes shown as mean ± standard error of the mean (SEM)

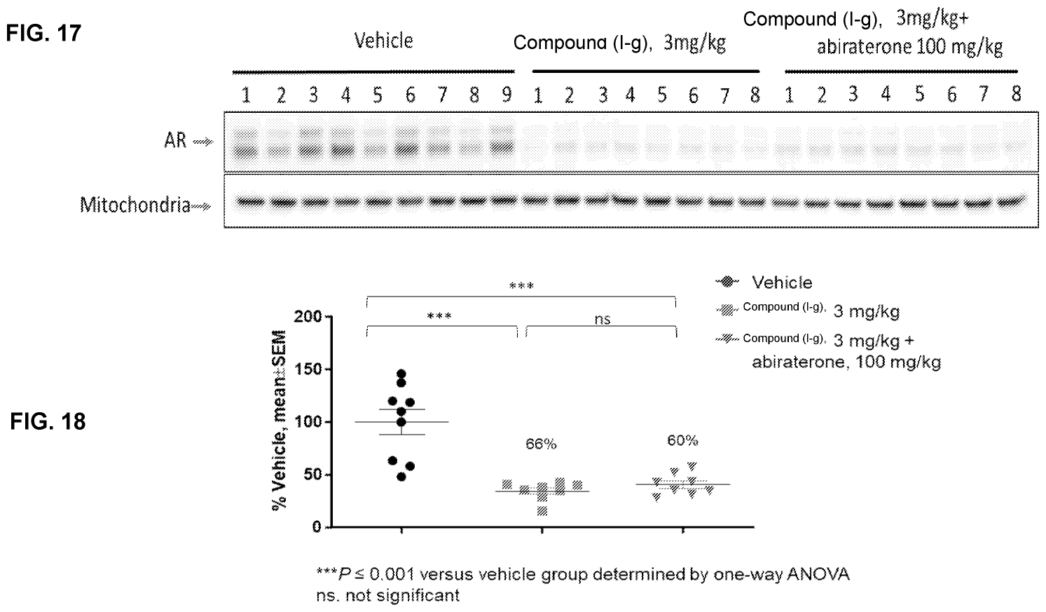

In order to evaluate the pharmacodynamic response, tumors were excised at the end of the Stage I and were subjected to analysis by Western blotting for androgen receptor (AR) levels. Significant (>60%) AR degradation was observed with 3 mg/kg Compound (I-g) as a single agent. Notably, the combination exhibited comparable AR reduction as with single agent Compound (I-g) (FIG. 17 and FIG. 18). This result suggests that the enhanced anti-tumor effect in vivo was driven by the combined pharmacological effect of both agents.

Study Design—VCaP Xenograft models: To generate castration resistant prostate cancer models, human prostate cancer VCaP (ATCC CRL®-2876®) cells (3×10⁶) were suspended in phosphate buffered saline with 75% Matrigel (BD Biosciences) and were implanted subcutaneously into the dorsal flank of 6-8-week-old CB17/scid male mice (Charles River). When tumors became palpable, mice were surgically castrated and followed for tumor growth. When tumors reached approximately 150 mm³, the mice were randomized and dosed with Compound (I-g), abiraterone, or a combination of both agents by oral gavage once daily for 43 days. Compound (I-g) and abiraterone were prepared in 5% dimethyl sulfoxide (DMSO)/95% (2% Tween80/PEG400) and 5% DMSO/95%(1% carboxymethyl cellulose/1% Tween80 in water), respectively. Change in tumor volume was measured using digital calipers and body weight of mice was measured twice weekly. Tumor volume was calculated with the formula (L×W²)/2, where L=length of tumor and W=width. At the conclusion of the Stage I portion of the study, animals dosed with vehicle, Compound (I-g), or the combination of Compound (I-g) and abiraterone were euthanized and tumors were removed, weighed, and frozen on dry ice before western blot analysis. TGI was calculated with the formula (where tumor volume is in mm³):

$$TGI ( \% ) = [ 1 - \frac{( Tumor volume , compound , Day X ) - ( Tumor volume , compound , Day 0 )}{( Tumor volume , vehicle or Abi ( Stage III ) , Day X ) - ( Tumor volume , vehicle or Abi ( Stage III ) , Day 0 )} ] \times 100.$$

For the Stage II portion of the study, animals in the abiraterone group were continuously dosed abiraterone 100 mg/kg once daily until tumors reached an average size of approximately 350 mm³. In the Stage III portion of the study, the animals were then randomized into two groups of 4 mice and dosed with abiraterone at 100 mg/kg or Compound (I-g) at 10 mg/kg once daily for 19 days.

Frozen prostate cell xenograft tumors were used to generate protein lysates. The tumor lysates were probed via western blot with antibodies against human AR (Cell Signaling Technology #5153) and human mitochondria (Abcam 92824). Mitochondria specific antibody served as a total protein loading control. Quantification of the western blot utilized all tumors in each arm.

Statistical analyses were performed using one-way analysis of variance (ANOVA) with GraphPad Prism to show statistically significant differences between the means of the vehicle-treated group and the treatment group.

### Example 11—Pharmacokinetic Data—Oral Administration of Compound (I-g) in Combination with Abiraterone

Preliminary PK data from study of Compound (I-g) in combination with abiraterone is presented below in Tables 4-6.

In vitro data suggests Compound (I-g) induces CYP3A4, a key enzyme involved in the metabolism of abiraterone and may consequently reduce abiraterone exposure. Preliminary results indicate a reduction in the average abiraterone AUC exposure of 22% when 1000 mg of abiraterone is given with 420 mg of Compound (I-g) (Table 4). The PK data of Compound (I-g) is included in Table 5.

The potential for DDI was also assessed. Preliminary results indicate the adjusted geometric mean ratios of abiraterone administered with Compound (I-g) vs. abiraterone alone are within the standard range [80-125%] for relative bioavailability; the results between both treatment periods are comparable and no significant DDI was observed (Table 6).

TABLE 4
Preliminary Abiraterone Pharmacokinetic Data for Study
of Compound (I-g) in Combination with Abiraterone.

| | | AUC₀₋ₜₐᵤ | AUCₗₐₛₜ | Cₘₐₓ | Tₘₐₓ |
|---|---|---|---|---|---|
| Treatment | | (ng-h/mL) | (ng-h/mL) | (ng/mL) | (h) |
| Abiraterone | n | 6 | 6 | 6 | 6 |
| alone (N = 6) | mean | 536 | 388 | 108 | 2 |
| | CV[%] | 54.3 | 53.3 | 69.1 | (2.00, 2.00) |
| Abiraterone | n | 6 | 6 | 6 | 6 |
| with Compound | mean | 417 | 335 | 117 | 2 |
| (I-g) (N = 6) | CV[%] | 33.8 | 38.5 | 40.5 | (1.00, 2.00) |

AUCₗₐₛₜ = area under the concentration-time curve from time zero to the last measurement; AUC₀₋ₜₐᵤ = area under the concentration-time curve from time zero during a dosing interval, calculated by linear up/log down trapezoidal summation; Cₘₐₓ = maximum concentration; CRO = clinical research organization; CV = coefficient of variation; PK = pharmacokinetic; Tₘₐₓ = time to maximum plasma concentration. Tₘₐₓ is reported as median (min, max). Time of AUCₗₐₛₜ is 8 hours.

TABLE 5
Preliminary Compound (I-g) Pharmacokinetic Data for Study
of Compound (I-g) in Combination with Abiraterone.

| | | AUC₀₋ₜₐᵤ | AUCₗₐₛₜ | Cₘₐₓ | Tₘₐₓ |
|---|---|---|---|---|---|
| Treatment | | (ng-h/mL) | (ng-h/mL) | (ng/mL) | (h) |
| Abiraterone with | n | 6 | 6 | 6 | 6 |
| Compound (I-g) | mean | 9,120 | 3,030 | 451 | 6 |
| (N = 6) | CV[%] | 38.6 | 43.6 | 41.6 | (0.00, 8.00) |

AUCₗₐₛₜ = area under the concentration-time curve from time zero to the last measurement; AUC₀₋ₜₐᵤ = area under the concentration-time curve from time zero during a dosing interval, calculated by linear up/log down trapezoidal summation; Cₘₐₓ = maximum concentration; mPC = metastatic prostate cancer; PK = pharmacokinetic; Tₘₐₓ = time to maximum plasma concentration. Tₘₐₓ is reported as median (min, max). Time of AUCₗₐₛₜ is 8 hours.

TABLE 6
Adjusted By-Treatment Geometric Means and Relative Bioavailability
Comparison of Abiraterone Administered with Compound (I-g)
(“T”) Relative to Abiraterone Alone (“R)”.

| | Abiraterone + | | | | | |
|---|---|---|---|---|---|---|
| | Compound (I-g) | Abiraterone | | 90% Confidence | | |
| | (T) N = 6 | (R) N = 6 | | Interval | | |
| | Adjusted | Adjusted | | Lower | Upper | Intra- |
| PK | gMean (SE | gMean (SE | Ratio T | Limit | Limit | subject |
| Parameter | on ln scale) | on ln scale) | to R (%) | (%) | (%) | CV (%) |
| Cₘₐₓ | 108.96 (0.236) | 88.20 (0.236) | 123.54 | 84.05 | 181.58 | 34.03 |
| (ng/mL) | | | | | | |
| AUCₗₐₛₜ | 316.90 (0.177) | 348.71 (0.177) | 90.88 | 65.83 | 125.46 | 28.26 |
| (h*ng/mL) | | | | | | |
| AUC₀₋ₜₐᵤ | 398.09 (0.179) | 476.14 (0.179) | 83.61 | 58.55 | 119.39 | 31.35 |
| (h*ng/mL) | | | | | | |

AUCₗₐₛₜ = area under the concentration-time curve from time zero to the last measurement;
AUC₀₋ₜₐᵤ = area under the concentration-time curve from time zero during a dosing interval, calculated by linear up/log down trapezoidal summation;
Cₘₐₓ = maximum concentration;
CV = coefficient of variation;
N = No. of subjects receiving respective treatment in the Pharmacokinetic Analysis Population;
PK = pharmacokinetic;
SE = Standard Error

### Example 12—Efficacy of Compound (I-g) in a Prostate Cancer Patient-Derived Xenograft Mouse Model

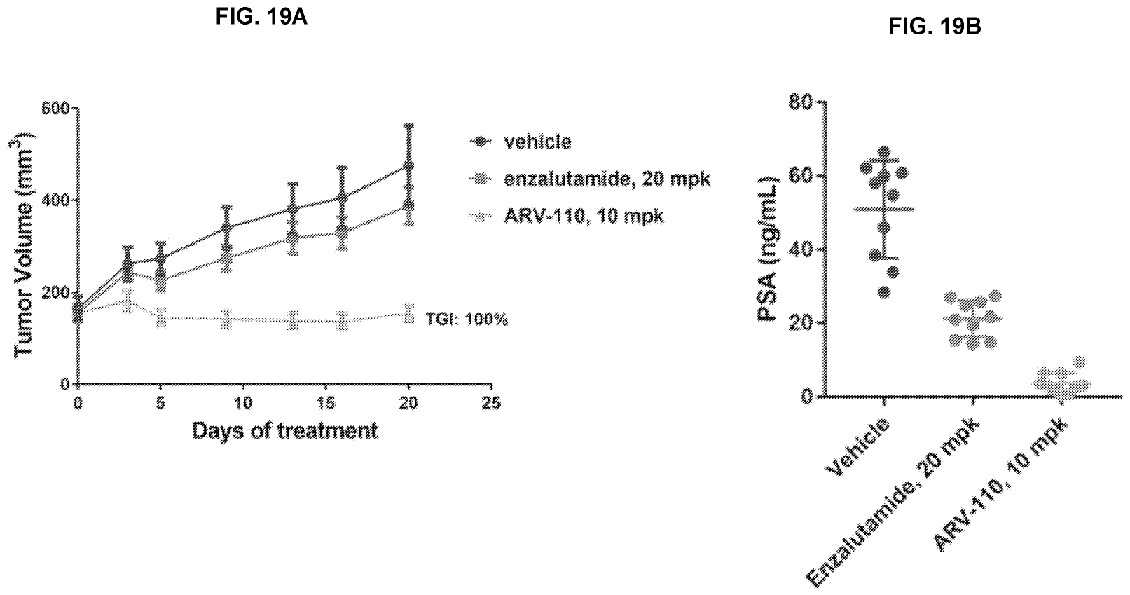

The effect of Compound (I-g) (10 mg/kg/day, PO) and enzalutamide (20 mg/kg/day, PO) was evaluated in a patient-derived xenograft (PDX) model TM00298 (Jackson Labs). This PDX was derived from a primary tumor located in the prostate gland of patient and has been shown to express AR and PSA. A published report demonstrated no TGI in this model upon enzalutamide treatment, although modest PSA reductions were observed (Jin 2017). The TM00298 PDX was grown in intact (non-castrate) male NSG (NOD scid IL2Ry) mice. As shown in FIG. 19A, after 20 days of treatment Compound (I-g) demonstrated 100% TGI, whereas no significant TGI was observed with enzalutamide consistent with the published report by Jin et al. (Jin 2017). Plasma PSA levels were lowered following enzalutamide treatment (58% reduction); however, a 93% reduction of plasma PSA was observed with Compound (I-g) treatment. (FIG. 19B.) The difference between Compound (I-g) and enzalutamide PSA reduction is statistically significant. These data demonstrate that Compound (I-g) is active in a prostate cancer PDX model that is largely insensitive to enzalutamide.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

### ENUMERATED EMBODIMENTS

The aspects of the present disclosure are further described with reference to the following numbered embodiments:
A1. A method of treating prostate cancer in a subject in need thereof, comprising:
(i) oral, once daily administration of about 35 mg to about 500 mg of a compound of Formula (I-g):

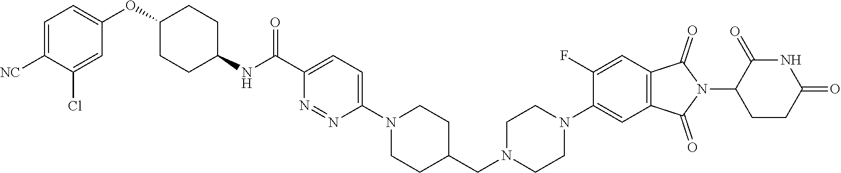 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof; and
(ii) oral, once daily administration of about 1000 mg abiraterone acetate, or a pharmaceutically acceptable salt thereof.
A2. A method of treating prostate cancer in a subject in need thereof, comprising:
(i) oral, once daily administration of about 35 mg to about 500 mg of a compound of Formula (I-g):

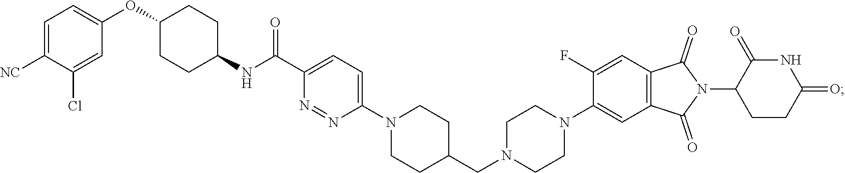 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(ii) oral, once daily administration of about 1000 mg abiraterone acetate or a pharmaceutically acceptable salt thereof.
A3. A combined preparation of:
(a) a compound of Formula (I-g):

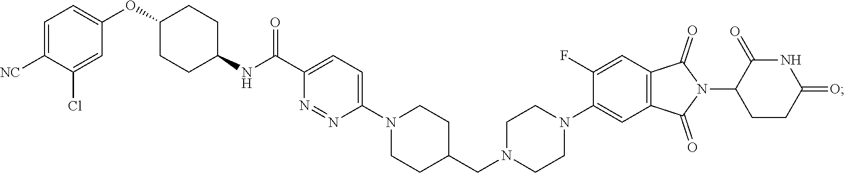 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

and
(b) abiraterone acetate, or a pharmaceutically acceptable salt thereof; for simultaneous, separate or sequential use in a method of treating prostate cancer in a subject.
A4. The combined preparation of Embodiment A3, wherein about 35 mg to about 500 mg of the compound of Formula (I-g) is orally administered to the subject once daily.
A5. The combined preparation for use of Embodiment A3, wherein the method comprises:
(i) oral, once daily administration of about 35 mg to about 500 mg of the compound of Formula (I-g) or the pharmaceutically acceptable salt thereof; and
(ii) oral, once daily administration of about 1000 mg abiraterone acetate, or the pharmaceutically acceptable salt thereof.
A6. A compound of Formula (I-g):

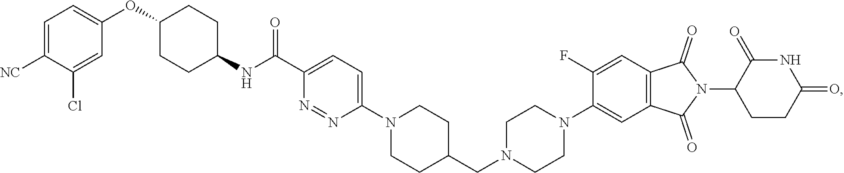 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

for use in a method of treating prostate cancer in a subject, wherein the method comprises:
(i) oral, once daily administration of about 35 mg to about 500 mg of the compound of Formula (I-g) or the pharmaceutically acceptable salt thereof; and
(ii) oral, once daily administration of about 1000 mg abiraterone acetate, or a pharmaceutically acceptable salt thereof.
B1. The method, combined preparation, or compound for use of any one of Embodiments A1-A6, wherein the method further comprises administering a corticosteroid to the subject.
B2. The method, combined preparation, or compound for use of any one of Embodiments A1-A6, wherein the method further comprises administering prednisone to the subject.
B3. The method, combined preparation, or compound for use of any one of Embodiments A1-A6, wherein the method further comprises administering prednisolone to the subject.
C1. The method, combined preparation, or compound for use of Embodiment B1 wherein about 0.5 mg, about 1 mg, about 1.5 mg, about 2 mg, about 2.5 mg, about 3 mg, about 3.5 mg, about 4 mg, about 4.5 mg, or about 5 mg of the corticosteroid is administered to the subject.
C2. The method, combined preparation, or compound for use of Embodiment B1 wherein about 0.5 mg of the corticosteroid is administered to the subject.
C3. The method, combined preparation, or compound for use of Embodiment B1 wherein about 1 mg of the corticosteroid is administered to the subject.
C4. The method, combined preparation, or compound for use of Embodiment B1 wherein about 1.5 mg of the corticosteroid is administered to the subject.
C5. The method, combined preparation, or compound for use of Embodiment B1 wherein about 2 mg of the corticosteroid is administered to the subject.
C6. The method, combined preparation, or compound for use of Embodiment B1 wherein about 2.5 mg of the corticosteroid is administered to the subject.
C7. The method, combined preparation, or compound for use of Embodiment B1 wherein about 3 mg of the corticosteroid is administered to the subject.
C8. The method, combined preparation, or compound for use of Embodiment B1 wherein about 3.5 mg of the corticosteroid is administered to the subject.
C9. The method, combined preparation, or compound for use of Embodiment B1 wherein about 4 mg of the corticosteroid is administered to the subject.
C10. The method, combined preparation, or compound for use of Embodiment B1 wherein about 4.5 mg of the corticosteroid is administered to the subject.
C11. The method, combined preparation, or compound for use of Embodiment B1 wherein about 5 mg of the corticosteroid is administered to the subject.
D1. The method, combined preparation, or compound for use of any one of Embodiments B1 or C1-C11 wherein the corticosteroid is orally administered to the subject once daily.
D2. The method, combined preparation, or compound for use of any one of Embodiments B1 or C1-C11 wherein the corticosteroid is orally administered to the subject twice daily.
E1. The method, combined preparation, or compound for use of any one of Embodiments B1 or C1-D2 wherein the corticosteroid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclomethasone.
E2. The method, combined preparation, or compound for use of any one of Embodiments B1 or C1-D2 wherein the corticosteroid is prednisone.
E3. The method, combined preparation, or compound for use of any one of Embodiments B1 or C1-D2 wherein the corticosteroid is prednisolone.
F1. The method, combined preparation, or compound for use of any one of Embodiments A1-E3 wherein about 1,000 mg of abiraterone acetate is orally administered to the subject once daily.
F2. The method, combined preparation, or compound for use of any one of Embodiments A1-E3 wherein between about 500 mg and about 1500 mg of abiraterone acetate is orally administered to the subject once daily.
G1. The method, combined preparation, or compound for use of any one of Embodiments A1-F2, wherein about 35 mg to about 100 mg of the compound of Formula (I-g) is orally administered to the subject once daily.
G2. The method, combined preparation, or compound for use of any one of Embodiments A1-F2, wherein about 100 mg to about 200 mg of the compound of Formula (I-g) is orally administered to the subject once daily.
G3. The method, combined preparation, or compound for use of any one of Embodiments A1-F2, wherein about 200 mg to about 300 mg of the compound of Formula (I-g) is orally administered to the subject once daily.
G4. The method, combined preparation, or compound for use of any one of Embodiments A1-F2, wherein about 300 mg to about 400 mg of the compound of Formula (I-g) is orally administered to the subject once daily.
G5. The method, combined preparation, or compound for use of any one of Embodiments A1-F2, wherein about 400 mg to about 500 mg of the compound of Formula (I-g) is orally administered to the subject once daily.
G6. The method, combined preparation, or compound for use of any one of Embodiments A1-F2, wherein about 35 mg of the compound of Formula (I-g) is orally administered to the subject once daily.
G7. The method, combined preparation, or compound for use of any one of Embodiments A1-F2, wherein about 100 mg of the compound of Formula (I-g) is orally administered to the subject once daily.
G8. The method, combined preparation, or compound for use of any one of Embodiments A1-F2, wherein about 140 mg of the compound of Formula (I-g) is orally administered to the subject once daily.
G9. The method, combined preparation, or compound for use of any one of Embodiments A1-F2, wherein about 150 mg of the compound of Formula (I-g) is orally administered to the subject once daily.
G10. The method, combined preparation, or compound for use of any one of Embodiments A1-F2, wherein about 200 mg of the compound of Formula (I-g) is orally administered to the subject once daily.
G11. The method, combined preparation, or compound for use of any one of Embodiments A1-F2, wherein about 250 mg of the compound of Formula (I-g) is orally administered to the subject once daily.
G12. The method, combined preparation, or compound for use of any one of Embodiments A1-F2, wherein about 300 mg of the compound of Formula (I-g) is orally administered to the subject once daily.
G13. The method, combined preparation, or compound for use of any one of Embodiments A1-F2, wherein about 350 mg of the compound of Formula (I-g) is orally administered to the subject once daily.
G14. The method, combined preparation, or compound for use of any one of Embodiments A1-F2, wherein about 400 mg of the compound of Formula (I-g) is orally administered to the subject once daily.
G15. The method, combined preparation, or compound for use of any one of Embodiments A1-F2, wherein about 420 mg of the compound of Formula (I-g) is orally administered to the subject once daily.
G16. The method, combined preparation, or compound for use of any one of Embodiments A1-F2, wherein about 450 mg of the compound of Formula (I-g) is orally administered to the subject once daily.
G17. The method, combined preparation, or compound for use of any one of Embodiments A1-F2, wherein about 500 mg of the compound of Formula (I-g) is orally administered to the subject once daily.
H1. The method, combined preparation, or compound for use of any one of Embodiments A1-G17 wherein the compound of Formula (I-g) is formulated as a tablet.
H2. The method of Embodiment H1, wherein the tablet comprises the compound of Formula (I-g) and, optionally, one or more of the following: emulsifier; surfactant; binder; disintegrant; glidant; and lubricant.
I1. The method, combined preparation, or compound for use of any one of Embodiments A1-H2, wherein the subject is in a fed state.
I2. The method, combined preparation, or compound for use of any one of Embodiments A1-H2, wherein the subject is in a fasted state.
J1. The method, combined preparation, or compound for use of any one of Embodiments A1-I1, wherein the prostate cancer is castrate-resistant prostate cancer.
J2. The method, combined preparation, or compound for use of any one of Embodiments A1-I1, wherein the prostate cancer is metastatic prostate cancer.
J3. The method, combined preparation, or compound for use of any one of Embodiments A1-I1, wherein the prostate cancer is metastatic castrate-resistant prostate cancer.
J4. The method, combined preparation, or compound for use of any one of Embodiments A1-I1, wherein the prostate cancer is adenocarcinoma of the prostate.
J5. The method, combined preparation, or compound for use of any one of Embodiments A1-I1, wherein the prostate cancer is progressive metastatic castrate-resistant prostate cancer.
J6. The method, combined preparation, or compound for use of any one of Embodiments A1-I1, wherein the prostate cancer is castrate-sensitive prostate cancer.
J7. The method, combined preparation, or compound for use of any one of Embodiments A1-I1, wherein the prostate cancer is metastatic castrate-sensitive prostate cancer.
J8. The method, combined preparation, or compound for use of any one of Embodiments A1-I1, wherein the prostate cancer is prostate cancer naïve to novel hormonal agents (NHA).
J9. The method, combined preparation, or compound for use of any one of Embodiments A1-I1, wherein the prostate cancer is metastatic prostate cancer naïve to novel hormonal agents (NHA).
J10. The method, combined preparation, or compound for use of any one of Embodiments A1-I1, wherein the prostate cancer is castrate-resistant prostate cancer naïve to novel hormonal agents (NHA).
J11. The method, combined preparation, or compound for use of any one of Embodiments A1-I1, wherein the prostate cancer is castrate-sensitive prostate cancer naïve to novel hormonal agents (NHA).
J12. The method, combined preparation, or compound for use of any one of Embodiments A1-I1, wherein the prostate cancer is metastatic castrate-resistant prostate cancer naïve to novel hormonal agents (NHA).
J13. The method, combined preparation, or compound for use of any one of Embodiments A1-I1, wherein the prostate cancer is metastatic castrate-sensitive prostate cancer naïve to novel hormonal agents (NHA).
K1. The method, combined preparation, or compound for use of Embodiment J8, wherein the prostate cancer naïve to novel hormonal agents (NHA) has not been previously treated with a second generation antiandrogen.
K2. The method, combined preparation, or compound for use of Embodiment J9, wherein the metastatic prostate cancer naïve to novel hormonal agents (NHA) has not been previously treated with a second generation antiandrogen.
K3. The method, combined preparation, or compound for use of Embodiment J10, wherein the castrate-resistant prostate cancer naïve to novel hormonal agents (NHA) has not been previously treated with a second generation antiandrogen.
K4. The method, combined preparation, or compound for use of Embodiment J11, wherein the castrate-sensitive prostate cancer naïve to novel hormonal agents (NHA) has not been previously treated with a second generation antiandrogen.
K5. The method, combined preparation, or compound for use of Embodiment J12, wherein the metastatic castrate-resistant prostate cancer naïve to novel hormonal agents (NHA) has not been previously treated with a second generation antiandrogen.
K6. The method, combined preparation, or compound for use of Embodiment J13, wherein the metastatic castrate-sensitive prostate cancer naïve to novel hormonal agents (NHA) has not been previously treated with a second generation antiandrogen.
L1. The method, combined preparation, or compound for use of any one of Embodiments A1-K6, wherein the prostate cancer has not been previously treated with an androgen biosynthesis inhibitor or an androgen receptor blocker.
L2. The method, combined preparation, or compound for use of any one of Embodiments A1-K6, wherein the prostate cancer has not been previously treated with abiraterone acetate.
L3. The method, combined preparation, or compound for use of any one of Embodiments A1-K6, wherein the prostate cancer has not been previously treated with an androgen receptor blocker selected from enzalutamide, darolutamide, and apalutamide.
L4. The method, combined preparation, or compound for use of any one of Embodiments A1-K6, wherein the subject has not been previously administered an androgen biosynthesis inhibitor or an androgen receptor blocker.
L5. The method, combined preparation, or compound for use of any one of Embodiments A1-K6, wherein the subject has not been previously administered abiraterone acetate.
L6. The method, combined preparation, or compound for use of any one of Embodiments A1-K6, wherein the subject has not been previously administered an androgen receptor blocker selected from enzalutamide, darolutamide, and apalutamide.
M1. The method, combined preparation, or compound for use of any one of Embodiments A1-L6, wherein the compound of Formula (I-g) or pharmaceutically acceptable salt thereof and the abiraterone acetate or pharmaceutically acceptable salt thereof are administered simultaneously.
M2. The method, combined preparation, or compound for use of any one of Embodiments A1-L6, wherein the compound of Formula (I-g) or pharmaceutically acceptable salt thereof and the abiraterone acetate or pharmaceutically acceptable salt thereof are administered simultaneously in separate formulations.
M3. The method, combined preparation, or compound for use of any one of Embodiments A1-L6, wherein the compound of Formula (I-g) or pharmaceutically acceptable salt thereof and the abiraterone acetate or pharmaceutically acceptable salt thereof are administered simultaneously in the same formulation.
M4. The method, combined preparation, or compound for use of any one of Embodiments A1-L6, wherein the compound of Formula (I-g) or pharmaceutically acceptable salt thereof and the abiraterone acetate or pharmaceutically acceptable salt thereof are administered sequentially.
M5. The method, combined preparation, or compound for use of any one of Embodiments A1-L6, wherein the compound of Formula (I-g) or pharmaceutically acceptable salt thereof and the abiraterone acetate or pharmaceutically acceptable salt thereof are administered in temporal proximity.

What is claimed is:

1. A method of treating prostate cancer in a subject in need thereof, comprising:
(i) oral, once daily administration of about 35 mg to about 500 mg of a compound of Formula (I-g): 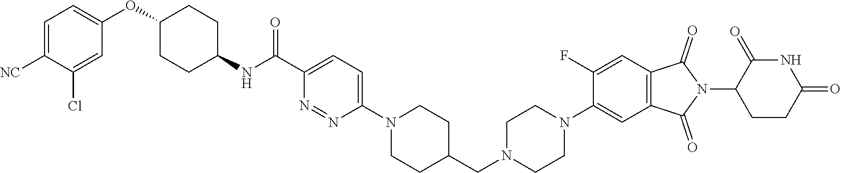 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]
or a pharmaceutically acceptable salt thereof; and
(ii) oral, once daily administration of about 1000 mg abiraterone, a pharmaceutically acceptable salt of abiraterone, abiraterone acetate, or a pharmaceutically acceptable salt of abiraterone acetate.

2. A method of treating prostate cancer in a subject in need thereof, comprising:
(i) oral, once daily administration of about 35 mg to about 500 mg of compound of Formula (I-g): 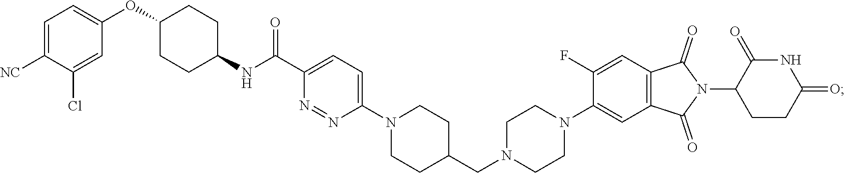 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]
and
(ii) oral, once daily administration of about 1000 mg abiraterone acetate.

3. The method of claim 1 or 2, further comprising administering a corticosteroid to the subject.

4. The method of claim 3, wherein about 0.5 mg, about 1 mg, about 1.5 mg, about 2 mg, about 2.5 mg, about 3 mg, about 3.5 mg, about 4 mg, about 4.5 mg, or about 5 mg of the corticosteroid is administered to the subject.

5. The method of claim 3 or 4, wherein the corticosteroid is orally administered to the subject once daily.

6. The method of claim 3 or 4, wherein the corticosteroid is orally administered to the subject twice daily.

7. The method of any one of claims 3-6, wherein the corticosteroid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclomethasone.

8. The method of any one of claims 3-7, wherein the corticosteroid is prednisone.

9. The method of any one of claims 3-7, wherein the corticosteroid is prednisolone.

10. The method of any one of claims 1-9, wherein 1,000 mg of abiraterone acetate is orally administered to the subject once daily.

11. The method of any one of claims 1-10, wherein about 35 mg to about 100 mg of the compound of Formula (I-g) is orally administered to the subject once daily.

12. The method of any one of claims 1-10, wherein about 100 mg to about 200 mg of the compound of Formula (I-g) is orally administered to the subject once daily.

13. The method of any one of claims 1-10, wherein about 200 mg to about 300 mg of the compound of Formula (I-g) is orally administered to the subject once daily.

14. The method of any one of claims 1-10, wherein about 300 mg to about 400 mg of the compound of Formula (I-g) is orally administered to the subject once daily.

15. The method of any one of claims 1-10, wherein about 400 mg to about 500 mg of the compound of Formula (I-g) is orally administered to the subject once daily.

16. The method of any one of claims 1-10, wherein about 35 mg of the compound of Formula (I-g) is orally administered to the subject once daily.

17. The method of any one of claims 1-10, wherein about 100 mg of the compound of Formula (I-g) is orally administered to the subject once daily.

18. The method of any one of claims 1-10, wherein about 140 mg of the compound of Formula (I-g) is orally administered to the subject once daily.

19. The method of any one of claims 1-10, wherein about 150 mg of the compound of Formula (I-g) is orally administered to the subject once daily.

20. The method of any one of claims 1-10, wherein about 200 mg of the compound of Formula (I-g) is orally administered to the subject once daily.

21. The method of any one of claims 1-10, wherein about 250 mg of the compound of Formula (I-g) is orally administered to the subject once daily.

22. The method of any one of claims 1-10, wherein about 300 mg of the compound of Formula (I-g) is orally administered to the subject once daily.

23. The method of any one of claims 1-10, wherein about 350 mg of the compound of Formula (I-g) is orally administered to the subject once daily.

24. The method of any one of claims 1-10, wherein about 400 mg of the compound of Formula (I-g) is orally administered to the subject once daily.

25. The method of any one of claims 1-10, wherein about 420 mg of the compound of Formula (I-g) is orally administered to the subject once daily.

26. The method of any one of claims 1-10, wherein about 450 mg of the compound of Formula (I-g) is orally administered to the subject once daily.

27. The method of any one of claims 1-10, wherein about 500 mg of the compound of Formula (I-g) is orally administered to the subject once daily.

28. The method of any one of claims 1-27, wherein the compound of Formula (I-g) is formulated as a tablet.

29. The method of claim 28, wherein the tablet comprises the compound of Formula (I-g) and, optionally, one or more of the following: emulsifier; surfactant; binder; disintegrant; glidant; and lubricant.

30. The method of any one of claims 1-29, wherein the subject is in a fed state.

31. The method of any one of claims 1-29, wherein the subject is in a fasted state.

32. The method of any one claims 1-31, wherein the prostate cancer is adenocarcinoma of the prostate.

33. The method of any one of claims 1-31, wherein the prostate cancer is metastatic prostate cancer.

34. The method of any one of claims 1-31, wherein the prostate cancer is castrate-resistant prostate cancer.

35. The method of any one of claims 1-31, wherein the prostate cancer is metastatic castrate-resistant prostate cancer.

36. The method of any one of claims 1-31, wherein the prostate cancer is progressive metastatic castrate-resistant prostate cancer.

37. The method of any one of claims 1-31, wherein the prostate cancer is castrate-sensitive prostate cancer.

38. The method of any one of claims 1-31, wherein the prostate cancer is metastatic castrate-sensitive prostate cancer.

39. The method of any one of claims 1-31, wherein the prostate cancer is prostate cancer naïve to novel hormonal agents (NHA).

40. The method of claim 39, wherein the prostate cancer naïve to novel hormonal agents (NHA) has not been previously treated with a second generation antiandrogen.

41. The method of any one of claims 1-31, wherein the prostate cancer is metastatic prostate cancer naïve to novel hormonal agents (NHA).

42. The method of the claim 41, wherein the metastatic prostate cancer naïve to novel hormonal agents (NHA) has not been previously treated with a second generation antiandrogen.

43. The method of any one of claims 1-31, wherein the prostate cancer is castrate-resistant prostate cancer naïve to novel hormonal agents (NHA).

44. The method of the claim 43, wherein the castrate-resistant prostate cancer naïve to novel hormonal agents (NHA) has not been previously treated with a second generation antiandrogen.

45. The method of any one of claims 1-31, wherein the prostate cancer is castrate-sensitive prostate cancer naïve to novel hormonal agents (NHA).

46. The method of claim 45, wherein the castrate-sensitive prostate cancer naïve to novel hormonal agents (NHA) has not been previously treated with a second generation antiandrogen.

47. The method of any one of claims 1-31, wherein the prostate cancer is metastatic castrate-resistant prostate cancer naïve to novel hormonal agents (NHA).

48. The method of claim 47, wherein the metastatic castrate-resistant prostate cancer naïve to novel hormonal agents (NHA) has not been previously treated with a second generation antiandrogen.

49. The method of any one of claims 1-31, wherein the prostate cancer is metastatic castrate-sensitive prostate cancer naïve to novel hormonal agents (NHA).

50. The method of claim 49, wherein the metastatic castrate-sensitive prostate cancer naïve to novel hormonal agents (NHA) has not been previously treated with a second generation antiandrogen.

51. The method of any one claims 1-50, wherein the prostate cancer has not been previously treated with an androgen biosynthesis inhibitor or an androgen receptor blocker.

52. The method of any one claims 1-50, wherein the prostate cancer has not been previously treated with abiraterone acetate.

53. The method of any one claims 1-50, wherein the prostate cancer has not been previously treated with an androgen receptor blocker selected from enzalutamide, darolutamide, and apalutamide.

54. The method of any one claims 1-50, wherein the prostate cancer has not been previously treated with enzalutamide.

55. The method of any one claims 1-50, wherein the subject has not been previously administered an androgen biosynthesis inhibitor or an androgen receptor blocker.

56. The method of any one of the claims 1-50, wherein the subject has not been previously administered abiraterone acetate.

57. The method of any one of claims 1-50, wherein the subject has not been previously administered an androgen receptor blocker selected from enzalutamide, darolutamide, and apalutamide.

58. The method of any one of claims 1-50, wherein the subject has not been previously administered enzalutamide.

59. A combined preparation of:
(a) the compound of Formula (I-g): 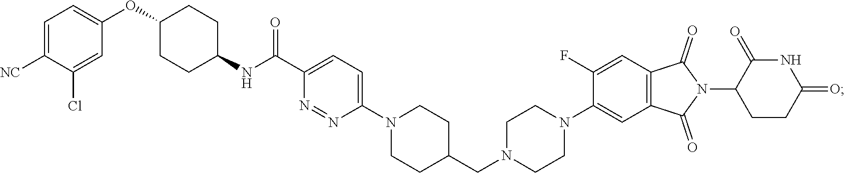 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]
and
(b) abiraterone acetate, or a pharmaceutically acceptable salt thereof;
for simultaneous, separate or sequential use in a method of treating prostate cancer in a subject.

60. The combined preparation for use of claim 59, wherein the method comprises:
(i) oral, once daily administration of about 35 mg to about 500 mg of the compound of Formula (I-g) or the pharmaceutically acceptable salt thereof; and
(ii) oral, once daily administration of about 1000 mg abiraterone acetate, or the pharmaceutically acceptable salt thereof.

61. The combined preparation for use of claim 59 or 60, wherein the method comprises:
(i) oral, once daily administration of about 35 mg to about 500 mg of the compound of Formula (I-g); and
(ii) oral, once daily administration of 1000 mg abiraterone acetate.

62. The compound of Formula (I-g):
for use in a method of treating prostate cancer in a subject, wherein the method comprises: 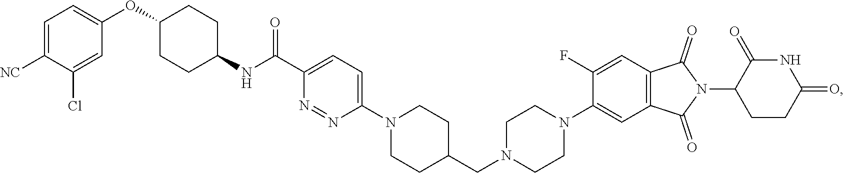 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]
(i) oral, once daily administration of about 35 mg to about 500 mg of the compound of Formula (I-g) or the pharmaceutically acceptable salt thereof; and
(ii) oral, once daily administration of about 1000 mg abiraterone acetate, or a pharmaceutically acceptable salt thereof.

63. The compound of Formula (I-g): 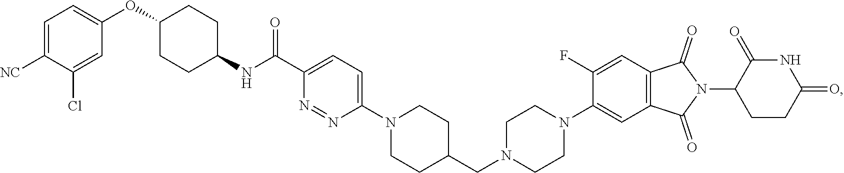 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]
for use in a method of treating prostate cancer in a subject, wherein the method comprises:
(i) oral, once daily administration of about 35 mg to about 500 mg of the compound of Formula (I-g); and
(ii) oral, once daily administration of 1000 mg abiraterone acetate.
